SEQUENCE LISTING (9 sequences)

SEQ ID NO: 1 - DNA, 2103 bp, Humicola grisea
atgcataccttctccaagctcctcgtcctgggctctgccgtccagtctgccctcgggcggcctcacggctcttcgcgtctccaggaacgcgctgccgttgataccttcatcaacaccgagaagcccatcgcatggaacaagctgctcgccaacatcggccctaacggcaaagccgctcccggtgccgccgccggcgttgtgattgccagcccttccaggacggaccctccttgtacgtggtggcatggaatggacccaagagactggttttagatgaaagagagtttctgctaaccgccacacccagacttcttcacctggacccgcgatgccgccctggtcctcaccggcatcatcgagtcccttggccacaactacaacaccaccctgcagaccgtcatccagaactacgtcgcgtcgcaggccaagctgcagcaggtctcgaacccctcgggaaccttcgccgacggctcgggtctcggtgaggccaagttcaatgtcgacctcactgccttcactggcgaatggggtcgccctcagagggacggcccgcccctgcgcgccatcgctctcatccagtacgccaagtggctgatcgccaacggctacaagagcacggccaagagcgtcgtctggcccgtcgtcaagaacgatctcgcctacacggcccagtactggaacgagaccggcttcgatctctgggaggaggtccccggcagctcgttctttaccatcgccagctctcacaggggtgagtcatttattgttcagtgttttctcattgaataattaccggaatgccactgacgccaaacagctctgactgagggtgcttacctcgccgctcagctcgacaccgagtgccgcgcctgcacgaccgtcgcccctcaggttctgtgcttccagcaggccttctggaactccaagggcaactatgtcgtctccaacagtaagatccctacaccaacaaaaaaaatcgaaaaggaacgttagctgacccttctagtcaacggcggcgagtatcgctccggcaaggacgccaactcgatcctggcgtccatccacaacttcgaccctgaggccggctgcgacaacctgaccttccagccctgcagcgagcgcgccctggccaaccacaaggcctatgtcgactcgttccgcaacctctacgccatcaacaagggcatcgcccagggcaaggccgttgccgtcggccgctactcggaggatgtctactacaacggcaacccgtggtacctggccaactttgccgccgccgagcagctctacgacgccatctacgtgtggaacaagcagggctccatcaccgtgacctcggtctccctgcccttcttccgcgaccttgtctcgtcggtcagcaccggcacctactccaagagcagctcgaccttcaccaacatcgtcaacgccgtcaaggcctacgccgacggcttcatcgaggtggcggccaagtacaccccgtccaacggcgcgctcgccgagcagtacgaccgcaacacgggcaagcccgactcggccgccgacctgacgtggtcgtactcggccttcctctcggccatcgaccgccgcgcgggtctcgtccccccgagctggcgggccagcgtggccaagagccagctgccgtccacctgctcgcgcatcgaggtcgccggcacctacgtcgccgccacgagcacctcgttcccgtccaagcagaccccgaacccctccgcggcgccctccccgtccccctacccgaccgcctgcgcggacgctagcgaggtgtacgtcaccttcaacgagcgcgtgtcgaccgcgtggggcgagaccatcaaggtggtgggcaacgtgccggcgctggggaactgggacacgtccaaggcggtgaccctgtcggccagcgggtacaagtcgaatgatcccctctggagcatcacggtgcccatcaaggcgacgggctcggccgtgcagtacaagtatatcaaggtcggcaccaacgggaagattacttgggagtcggaccccaacaggagcattaccctgcagacggcgtcgtctgcgggcaagtgcgccgcgcagacggtgaatgattcgtggcgttaa

SEQ ID NO: 2 - protein, 634 aa, Humicola grisea
MHTFSKLLVLGSAVQSALGRPHGSSRLQERAAVDTFINTEKPIAWNKLLANIGPNGKAAPGAAAGVVIASPSRTDPPYFFTWTRDAALVLTGIIESLGHNYNTTLQTVIQNYVASQAKLQQVSNPSGTFADGSGLGEAKFNVDLTAFTGEWGRPQRDGPPLRAIALIQYAKWLIANGYKSTAKSVVWPVVKNDLAYTAQYWNETGFDLWEEVPGSSFFTIASSHRALTEGAYLAAQLDTECRACTTVAPQVLCFQQAFWNSKGNYVVSNINGGEYRSGKDANSILASIHNFDPEAGCDNLTFQPCSERALANHKAYVDSFRNLYAINKGIAQGKAVAVGRYSEDVYYNGNPWYLANFAAAEQLYDAIYVWNKQGSITVTSVSLPFFRDLVSSVSTGTYSKSSSTFTNIVNAVKAYADGFIEVAAKYTPSNGALAEQYDRNTGKPDSAADLTWSYSAFLSAIDRRAGLVPPSWRASVAKSQLPSTCSRIEVAGTYVAATSTSFPSKQTPNPSAAPSPSPYPTACADASEVYVTFNERVSTAWGETIKVVGNVPALGNWDTSKAVTLSASGYKSNDPLWSITVPIKATGSAVQYKYIKVGTNGKITWESDPNRSITLQTASSAGKCAAQTVNDSWR

SEQ ID NO: 3 - protein, 604 aa, Humicola grisea
AAVDTFINTEKPIAWNKLLANIGPNGKAAPGAAAGVVIASPSRTDPPYFFTWTRDAALVLTGIIESLGHNYNTTLQTVIQNYVASQAKLQQVSNPSGTFADGSGLGEAKFNVDLTAFTGEWGRPQRDGPPLRAIALIQYAKWLIANGYKSTAKSVVWPVVKNDLAYTAQYWNETGFDLWEEVPGSSFFTIASSHRALTEGAYLAAQLDTECRACTTVAPQVLCFQQAFWNSKGNYVVSNINGGEYRSGKDANSILASIHNFDPEAGCDNLTFQPCSERALANHKAYVDSFRNLYAINKGIAQGKAVAVGRYSEDVYYNGNPWYLANFAAAEQLYDAIYVWNKQGSITVTSVSLPFFRDLVSSVSTGTYSKSSSTFTNIVNAVKAYADGFIEVAAKYTPSNGALAEQYDRNTGKPDSAADLTWSYSAFLSAIDRRAGLVPPSWRASVAKSQLPSTCSRIEVAGTYVAATSTSFPSKQTPNPSAAPSPSPYPTACADASEVYVTFNERVSTAWGETIKVVGNVPALGNWDTSKAVTLSASGYKSNDPLWSITVPIKATGSAVQYKYIKVGTNGKITWESDPNRSITLQTASSAGKCAAQTVNDSWR

SEQ ID NO: 4 - DNA, 1899 bp, Trichoderma reesei
atgcacgtcctgtcgactgcggtgctgctcggctccgttgccgttcaaaaggtcctgggaagaccaggatcaagcggtctgtccgacgtcaccaagaggtctgttgacgacttcatcagcaccgagacgcctattgcactgaacaatcttctttgcaatgttggtcctgatggatgccgtgcattcggcacatcagctggtgcggtgattgcatctcccagcacaattgacccggactactattacatgtggacgcgagatagcgctcttgtcttcaagaacctcatcgaccgcttcaccgaaacgtacgatgcgggcctgcagcgccgcatcgagcagtacattactgcccaggtcactctccagggcctctctaacccctcgggctccctcgcggacggctctggtctcggcgagcccaagtttgagttgaccctgaagcctttcaccggcaactggggtcgaccgcagcgggatggcccagctctgcgagccattgccttgattggatactcaaagtggctcatcaacaacaactatcagtcgactgtgtccaacgtcatctggcctattgtgcgcaacgacctcaactatgttgcccagtactggaaccaaaccggctttgacctctgggaagaagtcaatgggagctcattctttactgttgccaaccagcaccgagcacttgtcgagggcgccactcttgctgccactcttggccagtcgggaagcgcttattcatctgttgctccccaggttttgtgctttctccaacgattctgggtgtcgtctggtggatacgtcgactccaacatcaacaccaacgagggcaggactggcaaggatgtcaactccgtcctgacttccatccacaccttcgatcccaaccttggctgtgacgcaggcaccttccagccatgcagtgacaaagcgctctccaacctcaaggttgttgtcgactccttccgctccatctacggcgtgaacaagggcattcctgccggtgctgccgtcgccattggccggtatgcagaggatgtgtactacaacggcaacccttggtatcttgctacatttgctgctgccgagcagctgtacgatgccatctacgtctggaagaagacgggctccatcacggtgaccgccacctccctggccttcttccaggagcttgttcctggcgtgacggccgggacctactccagcagctcttcgacctttaccaacatcatcaacgccgtctcgacatacgccgatggcttcctcagcgaggctgccaagtacgtccccgccgacggttcgctggccgagcagtttgaccgcaacagcggcactccgctgtctgcgcttcacctgacgtggtcgtacgcctcgttcttgacagccacggcccgtcgggctggcatcgtgcccccctcgtgggccaacagcagcgctagcacgatcccctcgacgtgctccggcgcgtccgtggtcggatcctactcgcgtcccaccgccacgtcattccctccgtcgcagacgcccaagcctggcgtgccttccggtactccctacacgcccctgccctgcgcgaccccaacctccgtggccgtcaccttccacgagctcgtgtcgacacagtttggccagacggtcaaggtggcgggcaacgccgcggccctgggcaactggagcacgagcgccgccgtggctctggacgccgtcaactatgccgataaccaccccctgtggattgggacggtcaacctcgaggctggagacgtcgtggagtacaagtacatcaatgtgggccaagatggctccgtgacctgggagagtgatcccaaccacacttacacggttcctgcggtggcttgtgtgacgcaggttgtcaaggaggacacctggcagtcgtaa

SEQ ID NO: 5 - protein, 632 aa, Trichoderma reesei
MHVLSTAVLLGSVAVQKVLGRPGSSGLSDVTKRSVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 6 - protein, 599 aa, Trichoderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 7 - protein, 453 aa, Trichoderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTC

SEQ ID NO: 8 - protein, 604 aa, Rhizopus oryzae
MQLFNLPLKVSFFLVLSYFSLLVSAASIPSSASVQLDSYNYDGSTFSGKIYVKNIAYSKKVTVIYADGSDNWNNNGNTIAASYSAPISGSNYEYWTFSASINGIKEFYIKYEVSGKTYYDNNNSANYQVSTSKPTTTTATATTTTAPSTSTTTPPSRSEPATFPTGNSTISSWIKKQEGISRFAMLRNINPPGSATGFIAASLSTAGPDYYYAWTRDAALTSNVIVYEYNTTLSGNKTILNVLKDYVTFSVKTQSTSTVCNCLGEPKFNPDASGYTGAWGRPQNDGPAERATTFILFADSYLTQTKDASYVTGTLKPAIFKDLDYVVNVWSNGCFDLWEEVNGVHFYTLMVMRKGLLLGADFAKRNGDSTRASTYSSTASTIANKISSFWVSSNNWIQVSQSVTGGVSKKGLDVSTLLAANLGSVDDGFFTPGSEKILATAVAVEDSFASLYPINKNLPSYLGNSIGRYPEDTYNGNGNSQGNSWFLAVTGYAELYYRAIKEWIGNGGVTVSSISLPFFKKFDSSATSGKKYTVGTSDFNNLAQNIALAADRFLSTVQLHAHNNGSLAEEFDRTTGLSTGARDLTWSHASLITASYAKAGAPAA

SEQ ID NO: 9 - protein, 579 aa, Rhizopus oryzae
ASIPSSASVQLDSYNYDGSTFSGKIYVKNIAYSKKVTVIYADGSDNWNNNGNTIAASYSAPISGSNYEYWTFSASINGIKEFYIKYEVSGKTYYDNNNSANYQVSTSKPTTTTATATTTTAPSTSTTTPPSRSEPATFPTGNSTISSWIKKQEGISRFAMLRNINPPGSATGFIAASLSTAGPDYYYAWTRDAALTSNVIVYEYNTTLSGNKTILNVLKDYVTFSVKTQSTSTVCNCLGEPKFNPDASGYTGAWGRPQNDGPAERATTFILFADSYLTQTKDASYVTGTLKPAIFKDLDYVVNVWSNGCFDLWEEVNGVHFYTLMVMRKGLLLGADFAKRNGDSTRASTYSSTASTIANKISSFWVSSNNWIQVSQSVTGGVSKKGLDVSTLLAANLGSVDDGFFTPGSEKILATAVAVEDSFASLYPINKNLPSYLGNSIGRYPEDTYNGNGNSQGNSWFLAVTGYAELYYRAIKEWIGNGGVTVSSISLPFFKKFDSSATSGKKYTVGTSDFNNLAQNIALAADRFLSTVQLHAHNNGSLAEEFDRTTGLSTGARDLTWSHASLITASYAKAGAPAA

(19) United States
(12) Patent Application Publication
(10) Pub. No.: US 2014/0273145 A1
(43) Pub. Date: Sep. 18, 2014

(54) PRODUCTION OF ISOPRENE UNDER NEUTRAL pH CONDITIONS

(71) Applicants: THE GOODYEAR TIRE & RUBBER COMPANY, Akron, OH (US); Danisco US Inc., Palo Alto, CA (US)

(72) Inventors: Martien H. BERGSMA, Zoetermeer (NL); Anthony R. CALABRIA, Wilmington, DE (US); Gopal K. CHOTANI, Cupertino, CA (US); William A. CUEVAS, San Francisco, CA (US); Gang DUAN, Shanghai (CN); Sung Ho LEE, North Liberty, IA (US); Ying QIAN, Wuxi Jiangsu (CN); Vivek SHARMA, North Liberty, IA (US); Jayarama K. SHETTY, Pleasanton, CA (US); Bruce A. STROHM, Beloit, WI (US); Paula Johanna Maria TEUNISSEN, Saratoga, CA (US); Hongxian XU, Wuxi City (CN)

(73) Assignees: THE GOODYEAR TIRE & RUBBER COMPANY, Akron, OH (US); Danisco US Inc., Palo Alto, CA (US)

(21) Appl. No.: 14/218,720

(22) Filed: Mar. 18, 2014

Related U.S. Application Data
(63) Continuation of application No. 13/204,612, filed on Aug. 5, 2011, now abandoned.
(60) Provisional application No. 61/371,642, filed on Aug. 6, 2010.

(51) Int. Cl. C12P 5/00

(52) U.S. Cl. CPC C12P 5/007
USPC 435/167; 435/289.1

(57) ABSTRACT

Embodiments of the present disclosure relate to a process for producing isoprene from a starch substrate by saccharification and/or fermentation. The saccharification is effectively catalyzed by a glucoamylase at a pH in the range of 5.0 to 8.0. At a pH of 6.0 or above, the glucoamylase possesses at least 50% activity relative to its maximum activity. The saccharification and fermentation may be performed as a simultaneous saccharification and fermentation (SSF) process.

### CROSS-REFERENCE TO RELATED APPLICATIONS

This application is a continuation patent application of U.S. patent application Ser. No. 13/204,612, filed Aug. 5, 2011, which claims the priority benefit of U.S. Provisional Application No. 61/371,642, filed Aug. 6, 2010, the disclosures of which are herein incorporated by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 643842003001_Sequence_Listing.txt, date recorded: Mar. 18, 2014, size: 39,722 bytes).

### FIELD OF THE INVENTION

Glucoamylases capable of effectively hydrolyzing a starch substrate at a pH in the range of 5.0 to 8.0 are useful in simultaneous saccharification and fermentation (SSF) to produce an end product, such as isoprene.

### BACKGROUND

Industrial fermentations predominately use glucose as a feedstock for the production of a multitude of proteins, enzymes, alcohols, and other chemical end products. Typically, glucose is the product of starch processing, which is conventionally a two-step, enzymatic process that catalyzes the breakdown of starch, involving liquefaction and saccharification. During liquefaction, insoluble granular starch is slurried in water, gelatinized with heat, and hydrolyzed by a thermostable alpha-amylase. During saccharification, the soluble dextrins produced in liquefaction are further hydrolyzed by glucoamylases.

Glucoamylases are exo-acting carbohydrases, capable of hydrolyzing both the linear and branched glucosidic linkages of starch (e.g., amylose and amylopectin). Commercially, glucoamylases are typically used in the acidic pH ranges (pH less than 5.0) to produce fermentable sugars from the enzyme liquefied starch substrate. The fermentable sugars, e.g., low molecular weight sugars, such as glucose, may then be converted to fructose by other enzymes (e.g., glucose isomerases); crystallized; or used in fermentations to produce numerous end products (e.g., alcohols, monosodium glutamate, succinic acid, vitamins, amino acids, 1,3-propanediol, and lactic acid).

A system that combines (1) saccharification and (2) fermentation is known as simultaneous saccharification and fermentation (SSF). SSF replaces the classical double-step fermentation, i.e., production of fermentable sugars first and then conducting the fermentation process for producing the end product. In SSF, an inoculum can be added along with the starch hydrolyzing enzymes to concurrently saccharify a starch substrate and convert the saccharification products (i.e., fermentable sugars) to the desired end product. The inoculum is typically a microorganism capable of producing the end product. In addition to its various advantages, SSF is particularly promising where a high concentration substrate is present in a low reactor volume.

Isoprenoids, which are isoprene polymers, are used in pharmaceuticals, neutraceuticals, flavors, fragrances, and rubber products. Natural isoprenoid supplies, however, are limited, and commercial production of isoprenoids from their natural sources raises ecological concerns. Commercially viable quantities of isoprene, instead, can be obtained by direct isolation from petroleum C5 cracking fractions or by dehydration of C5 isoalkanes or isoalkenes. The C5 skeleton can also be synthesized from smaller subunits. Bacterial production of isoprene also has been described (Kuzma et al., Curr Microbiol, 30: 97-103, 1995; and Wilkins, Chemosphere, 32: 1427-1434, 1996). Isoprene production varies in amount with the phase of bacterial growth and the nutrient content of the culture medium. See e.g. U.S. Pat. No. 5,849,970, U.S. Published Patent Application Nos. 2009/0203102, 2010/0003716, 2010/0086978, and Wagner et al., J Bacteriol, 181:4700-4703, 1999.

What is needed is a simple, efficient method of producing isoprene in commercial quantities.

Throughout this specification, references are made to publications (e.g., scientific articles), patent applications, patents, etc., all of which are herein incorporated by reference in their entirety.

### BRIEF SUMMARY OF THE INVENTION

The invention provides, inter alia, for methods, compositions and systems for production of isoprene by a simultaneous saccharification and fermentation (SSF) process. The method takes advantage of the unique properties of certain glucoamylases. Glucoamylases such as Humicola grisea glucoamylase (HgGA), Trichoderma reesei glucoamylase (TrGA), and Rhizopus sp. glucoamylase (RhGA) display different pH profiles from other known glucoamylases, such as glucoamylases (GAs) from Aspergillus niger (AnGA) and Talaromyces emersonii (TeGA). At a pH of 6.0 or above, both HgGA and TrGA retain at least 50% of the activity relative to the maximum activity at pH 4.25 or pH 3.75, respectively. These glucoamylases are capable of saccharifying a starch substrate effectively at a pH in the range of 5.0 to 8.0, where cells (e.g., bacterial cells) can efficiently ferment the saccharified starch to isoprene. This property enables HgGA and TrGA to be used in SSF to produce isoprene compositions from a starch substrate in commercial quantities.

Accordingly, in one aspect of the invention, the invention provides for methods for producing isoprene comprising culturing a host cell, which comprises a heterologous nucleic acid encoding an isoprene synthase polypeptide, and saccharifying and fermenting a starch substrate under simultaneous saccharification and fermentation (SSF) conditions in the presence of a glucoamylase, wherein the saccharification and fermentation are performed at pH 5.0 to 8.0, wherein the glucoamylase possesses at least 50% activity at pH 6.0 or above relative to its maximum activity, wherein the glucoamylase is selected from the group consisting of a parent Humicola grisea glucoamylase (HgGA) comprising SEQ ID NO: 3, a parent Trichoderma reesei glucoamylase (TrGA) comprising SEQ ID NO: 6, a parent Rhizopus sp. glucoamylase (RhGA) comprising SEQ ID NO: 9, and a variant thereof, and wherein the variant has at least 99% sequence identity to the parent glucoamylase.

In one embodiment, the variant has one amino acid modification compared to the parent glucoamylase. In another embodiment, the HgGA is SEQ ID NO: 3. In another embodiment, the HgGA is produced from a Trichoderma reesei host cell. In another embodiment, the TrGA is SEQ ID No: 6. In another embodiment, the RhGA is SEQ ID NO: 9. In another embodiment, the SSF is carried out at pH 6.0 to 7.5. In another embodiment, the SSF is carried out at pH 7.0 to 7.5. In another embodiment, the SSF process is carried out at pH 7.0 to 7.5. In another embodiment, the SSF is performed at a temperature in a range of about 30° C. to about 60° C. In another embodiment, the SSF is performed at a temperature in a range of about 40° C. to about 60° C. In another embodiment, the starch substrate is about 15% to 50% dry solid (DS). In another embodiment, the starch substrate is about 15% to 30% dry solid (DS). In another embodiment, the starch substrate is about 15% to 25% dry solid (DS). In another embodiment, the starch substrate is granular starch or liquefied starch. In another embodiment, the glucoamylase is dosed at a range of about 0.1 to about 2.0 GAU per gram of dry substance starch. In another embodiment, the glucoamylase is dosed at a range of about 0.2 to about 1.0 GAU per gram of dry substance starch. In another embodiment, the glucoamylase is dosed at a range of about 0.5 to 1.0 GAU per gram of dry substance starch. In another embodiment, alpha-amylase is further added to any of the embodiments herein. In another embodiment, the alpha-amylase is from a Bacillus species, or a variant thereof. In another embodiment, the alpha-amylase is a Bacillus subtilis alpha-amylase (AmyE), a Bacillus amyloliquefaciens alpha-amylase, a Bacillus licheniformis alpha-amylase, a Bacillus stearothermophilus alpha-amylase, or a variant thereof. In another embodiment, the starch substrate is from corn, wheat, rye, barley, sorghum, cassava, tapioca, and any combination thereof. In another embodiment, the heterologous nucleic acid is operably linked to a promoter and wherein the production of isoprene by the cells is greater than about 5 g/L. In another embodiment, the isoprene synthase polypeptide is a plant isoprene synthase polypeptide. In another embodiment, the plant isoprene synthase is selected from the group consisting of Pueraria montana, Pueraria lobata, Populus alba, Populus nigra, Populus trichocarpa, Populus alba x tremula, Populus tremuloides and Quercus robur. In another embodiment, the host cells further comprise (i) one or more non-modified nucleic acids encoding feedback-resistant mevalonate kinase polypeptides or (ii) one or more additional copies of an endogenous nucleic acid encoding a feedback-resistant mevalonate kinase polypeptide. In another embodiment, the feedback-resistant mevalonate kinase is archaeal mevalonate kinase. In another embodiment, the mevalonate kinase polypeptide is selected from the group consisting of M. mazei, Lactobacillus mevalonate kinase polypeptide, Lactobacillus sakei mevalonate kinase polypeptide, yeast mevalonate kinase polypeptide, Streptococcus mevalonate kinase polypeptide, Streptococcus pneumoniae mevalonate kinase polypeptide, Streptomyces mevalonate kinase polypeptide, and Streptomyces CL190 mevalonate kinase polypeptide. In another embodiment, the host cells further comprise one or more heterologous nucleic acid encoding a mevalonate (MVA) pathway polypeptide or a DXP pathway polypeptide. In another embodiment, the host cell is selected from the group of bacterial cells, fungal cells, algal cells, plant cells, or cyanobacterial cells. In another embodiment, the bacterial cells are selected from the group consisting of gram-positive bacterial cells, gram-negative bacterial cells, E. coli, P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., and P. alcaligenes cells. In another embodiment, the fungal cells are selected from the group consisting of Aspergillus, yeast, Trichoderma, or Yarrowia cells. In another embodiment, the yeast is Saccharomyces sp., Schizosaccharomyces sp., Pichia sp., Candida sp. or Y. lipolytica cells. In another embodiment, the fungal cells are selected from the group consisting of A. oryzae, A. niger, S. cerevisiae, S. pombe, T. reesei, H. insolens, H. lanuginose, H. grisea, C. lucknowense, A. oryzae, A. niger, A sojae, A. japonicus, A. nidulans, A. aculeatus, A. awamori, F. roseum, F. graminum F. cerealis, F. oxysporuim, F. venenatum, N. crassa, M. miehei, T. viride, F. oxysporum, and F. solan cells. In another embodiment, the plant cells are selected from the group consisting of: the family Fabaceae, the Faboideae subfamily, kudzu, poplar, Populus alba x tremula, Populus alba, aspen, Populus tremuloides, and Quercus robur cells. In another embodiment, the algal cells are selected from the group consisting of: green algae, red algae, glaucophytes, chlorarachniophytes, euglenids, chromista, and dinoflagellates. In another embodiment, the isoprene is produced in the gas phase and (a) wherein the gas phase comprises greater than or about 9.5% (volume) oxygen, and the concentration of isoprene in the gas phase is less than the lower flammability limit or greater than the upper flammability limit or (b) the concentration of isoprene in the gas phase is less than the lower flammability limit or greater than the upper flammability limit, and the cells produce greater than about 400 nmole/g_{wcm}/hr of isoprene. In another embodiment, the host cells are grown under conditions that decouple isoprene production from cell growth. In another embodiment, the host cells are grown under limited glucose conditions.

In another embodiment, the invention provides for methods of processing starch comprising saccharifying a starch substrate to fermentable sugars at pH 5.0 to 8.0 in the presence of glucoamylase and at least one other enzyme, wherein the glucoamylase possesses at least 50% activity at pH 6.0 or above relative to its maximum activity, wherein the glucoamylase is selected from the group consisting of Humicola grisea glucoamylase (HgGA) comprising SEQ ID NO: 3, Trichoderma reesei glucoamylase (TrGA) comprising SEQ ID NO: 6, Rhizopus sp. glucoamylase (RhGA) comprising SEQ ID NO: 9, and a variant thereof, and wherein the variant has at least 99% sequence identity to a parent glucoamylase, and wherein the other enzyme is selected from the group consisting of proteases, pullulanases, isoamylases, cellulases, hemicellulases, xylanases, cyclodextrin glycotransferases, lipases, phytases, laccases, oxidases, esterases, cutinases, xylanases, and alpha-glucosidases.

In another embodiment, the invention provides for methods of processing starch comprising saccharifying a starch substrate to fermentable sugars at pH 5.0 to 8.0 in the presence of glucoamylase and at least one other non-starch polysaccharide hydrolyzing enzymes, wherein the glucoamylase possesses at least 50% activity at pH 6.0 or above relative to its maximum activity, wherein the glucoamylase is selected from the group consisting of Humicola grisea glucoamylase (HgGA) comprising SEQ ID NO: 3, Trichoderma reesei glucoamylase (TrGA) comprising SEQ ID NO: 6, Rhizopus sp. glucoamylase (RhGA) comprising SEQ ID NO: 9, and a variant thereof, and wherein the variant has at least 99% sequence identity to a parent glucoamylase, and wherein the non-starch polysaccharide hydrolyzing enzymes is selected from the group consisting of cellulases, hemicellulases and pectinases.

In another aspect, the invention provide for systems for producing isoprene comprising (i) a bioreactor within which saccharification and fermentation are performed at pH 5.0 to 8.0; (ii) a host cell comprising a heterologous nucleic acid encoding an isoprene synthase polypeptide; (iii) a glucoamylase that possesses at least 50% activity at pH 6.0 or above relative to its maximum activity, wherein the glucoamylase is selected from the group consisting of a parent Humicola grisea glucoamylase (HgGA) comprising SEQ ID NO: 3, a parent Trichoderma reesei glucoamylase (TrGA) comprising SEQ ID NO: 6, a parent Rhizopus p. glucoamylase (RhGA) comprising SEQ ID NO: 9, and a variant thereof, and wherein the variant has at least 99% sequence identity to the parent glucoamylase.

In another aspect, the invention provides for methods for producing isoprene comprising culturing a host cell, which comprises a heterologous nucleic acid encoding an isoprene synthase polypeptide, and saccharifying and fermenting a starch substrate under simultaneous saccharification and fermentation (SSF) conditions in the presence of a glucoamylase and at least one other enzyme, wherein the glucoamylase possesses at least 50% activity at pH 6.0 or above relative to its maximum activity, wherein the glucoamylase is selected from the group consisting of Humicola grisea glucoamylase (HgGA) comprising SEQ ID NO: 3, Trichoderma reesei glucoamylase (TrGA) comprising SEQ ID NO: 6, Rhizopus sp. glucoamylase (RhGA) comprising SEQ ID NO: 9, and a variant thereof, and wherein the variant has at least 99% sequence identity to a parent glucoamylase, and wherein the other enzyme is selected from the group consisting of proteases, pullulanasesi, isoamylases, cellulases, hemicellulases, xylanases, cyclodextrin glycotransferases, lipases, phytases, laccases, oxidases, esterases, cutinases, xylanases, and alpha-glucosidases.

In another aspect, the invention provides for methods for producing isoprene comprising culturing a host cell, which comprises a heterologous nucleic acid encoding an isoprene synthase polypeptide, and saccharifying and fermenting a starch substrate under simultaneous saccharification and fermentation (SSF) conditions in the presence of a glucoamylase and at least one other non-starch polysaccharide hydrolyzing enzymes, wherein the glucoamylase possesses at least 50% activity at pH 6.0 or above relative to its maximum activity, wherein the glucoamylase is selected from the group consisting of Humicola grisea glucoamylase (HgGA) comprising SEQ ID NO: 3, Trichoderma reesei glucoamylase (TrGA) comprising SEQ ID NO: 6, Rhizopus sp. glucoamylase (RhGA) comprising SEQ ID NO: 9, and a variant thereof, and wherein the variant has at least 99% sequence identity to a parent glucoamylase, and wherein the non-starch polysaccharide hydrolyzing enzymes is selected from the group consisting of cellulases, hemicellulases and pectinases.

In another aspect, the invention provides for compositions of isoprene produced by the methods and/or systems described herein.

### BRIEF DESCRIPTION OF THE DRAWINGS

The accompanying drawings are incorporated into the specification and provide non-limiting illustrations of various embodiments. In the drawings:

FIG. 1 depicts the pH profiles of HgGA, TrGA, AnGA, and TeGA, at 32° C. The pH profiles are presented as the percentage of the maximum activity under the saccharification conditions described in Example 1.

FIG. 2 depicts the presence of higher sugars after 48-hour saccharification reactions catalyzed by HgGA, TrGA, and AnGA. The saccharification reactions are described in Example 4.

FIG. 3 depicts scanning electron micrographs of corn, wheat, and cassava starch treated with HgGA and an alpha-amylase at pH 6.4. Starch samples are hydrolyzed by HgGA and an alpha-amylase under the conditions as described in Example 7.

FIG. 4 depicts the time course of accumulated glucose levels during isoprene production. The simultaneous saccharification and fermentation process was carried with TrGA and an alpha-amylase as described in Example 8.2.

FIG. 5 depicts the time course of isoprene titer. Isoprene production was achieved by the simultaneous saccharification and fermentation process with TrGA and an alpha-amylase as described in Example 8.2. The titer is defined as the amount of isoprene produced per liter of fermentation broth. The equation for calculating isoprene titer:

isoprene titer=∫(Instantaneous isoprene production rate,g/L/hr)dt from t=0 to 20 hrs[=]g/L broth(total isoprene produced over the time course per liter broth,g/L broth)

FIG. 6 depicts the time course of the carbon dioxide evolution rate (CER) or metabolic activity profile. Isoprene production was achieved by the simultaneous saccharification and fermentation process with TrGA and an alpha-amylase as described in Example 8.2.

FIG. 7 depicts the time course of the isoprene to carbon dioxide ratio in the gas stream exiting the bioreactor. The isoprene to carbon dioxide ratio is an indicator of product yield. Isoprene production was achieved by the simultaneous saccharification and fermentation process with TrGA and an alpha-amylase as described in Example 8.2.

FIG. 8 depicts the time course of accumulated glucose levels during isoprene production. The simultaneous saccharification and fermentation process was carried with HgGA as described in Example 8.3.

FIG. 9 depicts the time course of isoprene titer. Isoprene production was achieved by the simultaneous saccharification and fermentation process with HgGA as described in Example 8.3. The titer is defined as the amount of isoprene produced per liter of fermentation broth. The equation for calculating isoprene titer:

isoprene titer=∫(Instantaneous isoprene production rate,g/L/hr)dt from t=0 to 20 hrs[=]g/L broth.

FIG. 10 depicts the time course of the carbon dioxide evolution rate (CER) or metabolic activity profile. Isoprene production was achieved by the simultaneous saccharification and fermentation process with HgGA as described in Example 8.3.

FIG. 11 depicts the time course of the isoprene to carbon dioxide ratio in the gas stream exiting the bioreactor. The isoprene to carbon dioxide ratio is an indicator of product yield. Isoprene production was achieved by the simultaneous saccharification and fermentation process with HgGA as described in Example 8.3.

### DETAILED DESCRIPTION

The invention provides for methods and systems of producing isoprene using simultaneous saccharification and fermentation process and glucoamylases at neutral pH.

In one aspect, the present disclosure relates to the use of glucoamylases capable of effectively saccharifying a starch substrate at a neutral pH, for example, between pH 5.0 and 8.0, to provide an energy source for the biological production of isoprene. At a pH of 6.0 or above, the glucoamylases of the disclosed method retains at least about 50% activity relative to the maximum activity. The glucoamylases having these properties include, for example, HgGA, TrGA, and RhGA.

In some aspects, the embodiments of the present disclosure rely on routine techniques and methods used in the field of genetic engineering and molecular biology. The following resources include descriptions of general methodology useful in accordance with the invention: Sambrook et al., MOLECULAR CLONING: A LABORATORY MANUAL (2nd Ed., 1989); Kreigler, GENE TRANSFER AND EXPRESSION; A LABORATORY MANUAL (1990) and Ausubel et al., Eds. CURRENT PROTOCOLS IN MOLECULAR BIOLOGY (1994). Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the representative methods and materials are described. Numeric ranges are inclusive of the numbers defining the range. The headings provided herein are not limitations of the various aspects or embodiments, which can be had by reference to the specification as a whole.

### Definitions and Abbreviations

In accordance with this detailed description, the following abbreviations and definitions apply. It should be noted that as used herein, the singular forms “a,” “an,” and “the” include plural referents unless the context clearly dictates otherwise. Thus, for example, reference to “an enzyme” includes a plurality of such enzymes.

### DEFINITIONS

The term “isoprene” refers to 2-methyl-1,3-butadiene (CAS#78-79-5). It can be the direct and final volatile C5 hydrocarbon product from the elimination of pyrophosphate from 3,3-dimethylallyl pyrophosphate (DMAPP), and does not involve the linking or polymerization of [an] IPP molecule(s) to [a] DMAPP molecule(s). The term “isoprene” is not generally intended to be limited to its method of production unless indicated otherwise herein.

As used herein, “biologically produced isoprene” or “bioisoprene” is isoprene produced by any biological means, such as produced by genetically engineered cell cultures, natural microbials, plants or animals.

A “bioisoprene composition” refers to a composition that can be produced by any biological means, such as systems (e.g., cells) that are engineered to produce isoprene. It contains isoprene and other compounds that are co-produced (including impurities) and/or isolated together with isoprene. A bioisoprene composition usually contains fewer hydrocarbon impurities than isoprene produced from petrochemical sources and often requires minimal treatment in order to be of polymerization grade. A bioisoprene composition also has a different impurity profile from a petrochemically produced isoprene composition. As further detailed herein, a bioisoprene composition is distinguished from a petro-isoprene composition in that a bioisoprene composition is substantially free of any contaminating unsaturated C5 hydrocarbons that are usually present in petro-isoprene compositions, such as, but not limited to, 1,3-cyclopentadiene, trans-1,3-pentadiene, cis-1,3-pentadiene, 1,4-pentadiene, 1-pentyne, 2-pentyne, 3-methyl-1-butyne, pent-4-ene-1-yne, trans-pent-3-ene-1-yne, and cis-pent-3-ene-1-yne. If any contaminating unsaturated C5 hydrocarbons are present in the bioisoprene starting material described herein, they are present in lower levels than that in petro-isoprene compositions.

By “heterologous nucleic acid” is meant a nucleic acid whose nucleic acid sequence is not identical to that of another nucleic acid naturally found in the same host cell.

As used herein, “nucleotide sequence” or “nucleic acid sequence” refers to a sequence of genomic, synthetic, or recombinant origin and may be double-stranded or single-stranded, whether representing the sense or anti-sense strand. As used herein, the term “nucleic acid” may refer to genomic DNA, cDNA, synthetic DNA, or RNA. The residues of a nucleic acid may contain any of the chemically modifications commonly known and used in the art.

As used herein, “polypeptides” includes polypeptides, proteins, peptides, fragments of polypeptides, and fusion polypeptides. In some embodiments, the fusion polypeptide includes part or all of a first polypeptide (e.g., an isoprene synthase, DXS, IDI, or MVA pathway polypeptide or catalytically active fragment thereof) and may optionally include part or all of a second polypeptide (e.g., a peptide that facilitates purification or detection of the fusion polypeptide, such as a His-tag).

In some embodiments, the polypeptide is a heterologous polypeptide. By “heterologous polypeptide” is meant a polypeptide whose amino acid sequence is not identical to that of another polypeptide naturally expressed in the same host cell. In particular, a heterologous polypeptide is not identical to a wild-type nucleic acid that is found in the same host cell in nature.

“Isolated” means that the material is at least substantially free from at least one other component that the material is naturally associated and found in nature.

“Purified” means that the material is in a relatively pure state, e.g., at least about 90% pure, at least about 95% pure, at least about 98% pure, or at least about 99% pure.

“Oligosaccharide” means a carbohydrate molecule composed of 3-20 monosaccharides.

As used herein, “transformed cell” includes cells that have been transformed by use of recombinant DNA techniques. Transformation typically occurs by insertion of one or more nucleotide sequences into a cell. The inserted nucleotide sequence may be a heterologous nucleotide sequence, i.e., is a sequence that may not be natural to the cell that is to be transformed, such as a fusion protein.

As used herein, “starch” refers to any material comprised of the complex polysaccharide carbohydrates of plants, comprised of amylose and amylopectin with the formula (C₆H₁₀O₅)ₓ, wherein “X” can be any number. In particular, the term refers to any plant-based material including but not limited to grains, grasses, tubers and roots and more specifically wheat, barley, corn, rye, rice, sorghum, brans, cassava, millet, potato, sweet potato, and tapioca.

As used herein, “granular starch” refers to uncooked (raw) starch, which has not been subject to gelatinization.

As used herein, “starch gelatinization” means solubilization of a starch molecule to form a viscous suspension.

As used herein, “gelatinization temperature” refers to the lowest temperature at which gelatinization of a starch substrate occurs. The exact temperature depends upon the specific starch substrate and further may depend on the particular variety and the growth conditions of plant species from which the starch is obtained.

“DE” or “dextrose equivalent” is an industry standard for measuring the concentration of total reducing sugars, calculated as the percentage of the total solids that have been converted to reducing sugars. The granular starch that has not been hydrolyzed has a DE that is about zero (0), and D-glucose has a DE of about 100.

As used herein, “starch substrate” refers to granular starch or liquefied starch using refined starch, whole ground grains, or fractionated grains.

As used herein, “liquefied starch” refers to starch that has gone through solubilization process, for example, the conventional starch liquefaction process.

“Degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP4+ (>DP4) denotes polymers with a degree of polymerization of greater than four.

As used herein, “fermentable sugars” refer to saccharides that are capable of being metabolized under fermentation conditions. These sugars typically refer to glucose, maltose, and maltotriose (DP1, DP2 and DP3).

As used herein, “total sugar content” refers to the total sugar content present in a starch composition.

As used herein, “ds” refers to dissolved solids in a solution. The term “dry solids content (DS)” refers to the total solids of a slurry in % on a dry weight basis. The term “slurry” refers to an aqueous mixture containing insoluble solids.

As used herein, “starch-liquefying enzyme” refers to an enzyme that catalyzes the hydrolysis or breakdown of granular starch. Exemplary starch liquefying enzymes include alpha-amylases (EC 3.2.1.1).

“Amylase” means an enzyme that is, among other things, capable of catalyzing the degradation of starch. For example, β-Amylases, α-glucosidases (EC 3.2.1.20; α-D-glucoside glucohydrolase), glucoamylase (EC 3.2.1.3; α-D-(1→4)-glucan glucohydrolase), and product-specific amylases can produce malto-oligosaccharides of a specific length from starch.

“Alpha-amylases (EC 3.2.1.1)” refer to endo-acting enzymes that cleave α-D-(1→4) O-glycosidic linkages within the starch molecule in a random fashion. In contrast, the exo-acting amylolytic enzymes, such as beta-amylases (EC 3.2.1.2; α-D-(1→4)-glucan maltohydrolase) and some product-specific amylases like maltogenic alpha-amylase (EC 3.2.1.133) cleave the starch molecule from the non-reducing end of the substrate. These enzymes have also been described as those effecting the exo- or endohydrolysis of 1,4-α-D-glucosidic linkages in polysaccharides containing 1,4-α-linked D-glucose units. Another term used to describe these enzymes is glycogenase. Exemplary enzymes include alpha-1,4-glucan 4-glucanohydrolase.

As used herein, “glucoamylases” refer to the amyloglucosidase class of enzymes (EC 3.2.1.3, glucoamylase, α-1,4-D-glucan glucohydrolase). These are exo-acting enzymes that release glucosyl residues from the non-reducing ends of amylose and/or amylopectin molecules. The enzymes are also capably of hydrolyzing α-1,6 and α-1,3 linkages, however, at much slower rates than the hydrolysis of α-1,4 linkages.

As used herein, the term “non-starch polysaccharide hydrolyzing enzymes” are enzymes capable of hydrolyzing complex carbohydrate polymers such as cellulose, hemicellulose, and pectin. For example, cellulases (endo and exo-glucanases, beta glucosidase) hemicellulases (xylanases) and pectinases are non-starch polysaccharide hydrolyzing enzymes.

As used herein, “maximum activity” refers to the enzyme activity measured under the most favorable conditions, for example, at an optimum pH. As used herein, “optimum pH” refers to a pH value, under which the enzyme displays the highest activity with other conditions being equal. The “optimum pH” of HgGA and TrGA is shown in FIG. 1.

The phrase “mature form” of a protein or polypeptide refers to the final functional form of the protein or polypeptide. A mature form of a glucoamylase may lack a signal peptide and/or initiator methionine, for example. A mature form of a glucoamylase may be produced from its native host, for example, by endogenous expression. Alternatively, a mature form of a glucoamylase may be produced from a non-native host, for example, by exogenous expression. An exogenously expressed glucoamylase may have a varied glycosylation pattern compared to the endogenous expressed counterpart.

The term “parent” or “parent sequence” refers to a sequence that is native or naturally occurring.

As used herein, the terms “variant” is used in reference to glucoamylases that have some degree of amino acid sequence identity to a parent glucoamylase sequence. A variant is similar to a parent sequence, but has at least one substitution, deletion or insertion in their amino acid sequence that makes them different in sequence from a parent glucoamylase. In some cases, variants have been manipulated and/or engineered to include at least one substitution, deletion, or insertion in their amino acid sequence that makes them different in sequence from a parent. Additionally, a glucoamylase variant may retain the functional characteristics of the parent glucoamylase, e.g., maintaining a glucoamylase activity that is at least 50%, 60%, 70%, 80%, 90%, 95%, 98%, or 99% of that of the parent glucoamylase.

As used herein, “hydrolysis of starch” refers to the cleavage of glucosidic bonds with the addition of water molecules.

As used herein, “end product” or “desired end product” refers to a molecule or compound to which a starch substrate is converted into, by an enzyme and/or a microorganism.

As used herein, “contacting” or “admixing” refers to the placing of the respective enzyme(s) in sufficiently close proximity to the respective substrate to enable the enzyme(s) to convert the substrate to the end product. Those skilled in the art will recognize that mixing solutions of the enzyme with the respective substrates can affect contacting or admixing.

### ABBREVIATIONS

The following abbreviations apply unless indicated otherwise:

AkAA Aspergillus kawachii alpha-amylase

AmyE Bacillus subtilis alpha-amylase

AmyL Bacillus licheniformis alpha-amylase

AmyR SPEZYME® XTRA amylase

AmyS Geobacillus stearothermophilus alpha-amylase

AnGA Aspergillus niger glucoamylase

BAA bacterial alpha-amylase

cDNA complementary DNA

CER carbon dioxide evolution rate

DE Dextrose Equivalent

DI distilled, deionized

DMAPP 3,3-dimethylallyl pyrophosphate

DNA deoxyribonucleic acid

DP3 degree of polymerization with three subunits

DPn degree of polymerization with n subunits

DS or ds dry solid

dss dry solid starch

DXS 1-deoxy-D-xylulose-5-phosphate synthase

EC enzyme commission for enzyme classification

g gram

gpm gallon per minute

GAU glucoamylase units

HGA Humicola grisea glucoamylase

HgGA Humicola grisea glucoamylase

HPLC high pressure liquid chromatography

IPTG isopropyl-beta-D-1-thiogalactopyrano side

kg kilogram

MEP methylerythritol phosphate

MOPS 3-(N-morpholino)propanesulfonic acid

MT metric ton

MVA mevalonate

MW molecular weight

NCBI National Center for Biotechnology Information

nm nanometer

OD optical density

PCR polymerase chain reaction

PEG polyethylene glycol

pI isoelectric point

ppm parts per million

q.s. as much as suffices (quantum satis or quantum sufficit)

RhGA Rhizopus sp. glucoamylase

RNA ribonucleic acid

RO reverse osmosis

rpm revolutions per minute

slpm standard liters per minute

SSF simultaneous saccharification and fermentation

TeGA Talaromyces emersonii glucoamylase

TrGA Trichoderma reesei glucoamylase

w/v weight/volume

w/w weight/weight

wt wild-type

μL microliter

### Enzymes in Starch Processing

Glucoamylase Having the Desired pH Profile

Glucoamylases are produced by numerous strains of bacteria, fungi, yeast and plants. Many fungal glucoamylases are fungal enzymes that are extracellularly produced, for example from strains of Aspergillus (Svensson et al., Carlsberg Res. Commun. 48: 529-544 (1983); Boel et al., EMBO J. 3: 1097-1102 (1984); Hayashida et al., Agric. Biol. Chem. 53: 923-929 (1989); U.S. Pat. No. 5,024,941; U.S. Pat. No. 4,794,175 and WO 88/09795); Talaromyces (U.S. Pat. No. 4,247,637; U.S. Pat. No. 6,255,084; and U.S. Pat. No. 6,620,924); Rhizopus (Ashikari et al., Agric. Biol. Chem. 50: 957-964 (1986); Ashikari et al., App. Microbio. Biotech. 32: 129-133 (1989) and U.S. Pat. No. 4,863,864); Humicola (WO 05/052148 and U.S. Pat. No. 4,618,579); and Mucor (Houghton-Larsen et al., Appl. Microbiol. Biotechnol. 62: 210-217 (2003)). Many of the genes that code for these enzymes have been cloned and expressed in yeast, fungal and/or bacterial cells.

Commercially, glucoamylases are very important enzymes and have been used in a wide variety of applications that require the hydrolysis of starch (e.g., for producing glucose and other monosaccharides from starch). Glucoamylases are used to produce high fructose corn sweeteners, which comprise over 50% of the sweetener market in the United States. In general, glucoamylases may be, and commonly are, used with alpha-amylases in starch hydrolyzing processes to hydrolyze starch to dextrins and then glucose. The glucose may then be converted to fructose by other enzymes (e.g., glucose isomerases); crystallized; or used in fermentations to produce numerous end products (e.g., ethanol, citric acid, succinic acid, ascorbic acid intermediates, glutamic acid, glycerol, 1,3-propanediol and lactic acid).

The embodiments of the present disclosure utilize a glucoamylase capable of effectively saccharifying a starch substrate at a neutral pH, for example, between pH 5.0 and 8.0, 5.5 and 7.5, 6.0 and 7.5, 6.5 and 7.5, or 7.0 and 7.5. At a pH of 6.0 or above, the glucoamylase retains at least about 50%, about 51%, about 52%, about 53%, about 54%, or about 55% of the activity relative to the maximum activity. The glucoamylases having the desired pH profile include, but are not limited to, Humicola grisea glucoamylase (HgGA), Trichoderma reesei glucoamylase (TrGA), and Rhizopus sp. glucoamylase (RhGA).

HgGA may be the glucoamylase comprising the amino acid sequence of SEQ ID NO: 3, which is described in detail in U.S. Pat. Nos. 4,618,579 and 7,262,041. This HgGA is also described as a granular starch hydrolyzing enzyme (GSHE), because it is capable of hydrolyzing starch in granular form. The genomic sequence coding the HgGA from Humicola grisea var. thermoidea is presented as SEQ ID NO: 1, which contains three putative introns (positions 233-307, 752-817, and 950-1006). The native HgGA from Humicola grisea var. thermoidea has the amino acid sequence of SEQ ID NO: 2, which includes a signal peptide containing 30 amino acid residues (positions 1 to 30 of SEQ ID NO: 2). Cleavage of the signal peptide results in the mature HgGA having the amino acid sequence of SEQ ID NO: 3. The embodiments of the present disclosure also include a HgGA produced from a Trichoderma host cell, e.g., a Trichoderma reesei cell. See U.S. Pat. No. 7,262,041.

A typical TrGA is the glucoamylase from Trichoderma reesei QM6a (ATCC, Accession No. 13631). This TrGA comprising the amino acid sequence of SEQ ID NO: 6, which is described in U.S. Pat. No. 7,413,879, for example. The cDNA sequence coding the TrGA from Trichoderma reesei QM6a is presented as SEQ ID NO: 4. The native TrGA has the amino acid sequence of SEQ ID NO: 5, which includes a signal peptide containing 33 amino acid residues (positions 1 to 33 of SEQ ID NO: 4). See id. Cleavage of the signal peptide results in the mature TrGA having the amino acid sequence of SEQ ID NO: 6. See id. The catalytic domain of TrGA is presented as SEQ ID NO: 7. See id. The embodiments of the present disclosure also include an endogenously expressed TrGA. See id.

RhGA may be the glucoamylase from Rhizopus niveus or Rhizopus oryzae. See U.S. Pat. Nos. 4,514,496 and 4,092,434. The native RhGA from R. oryzae has the amino acid sequence of SEQ ID NO: 8, which includes a signal peptide containing 25 amino acid residues (positions 1 to 25 of SEQ ID NO:8). Cleavage of the signal peptide results in the mature RhGA having the amino acid sequence of SEQ ID NO: 9. A typical RhGA may be the glucoamylase having trade names CU.CONC (Shin Nihon Chemicals, Japan) or M1 (Biocon India, Bangalore, India).

Structure and Function

The glucoamylase of the embodiment of the present disclosure may also be a variant of HgGA, TrGA, or RhGA. The variant has at least 99% sequence identity to the parent glucoamylase. In some embodiments, the variant has at least 98%, at least 97%, at least 96%, at least 95%, at least 94%, at least 93%, at least 92%, at least 91%, or at least 90% sequence identity to the parent glucoamylase. Optionally, the variant has one, two, three, four, five, or six amino acids modification compared to the mature form of the parent glucoamylase. In other embodiments, the variant has at least 98%, 97%, 96%, 95%, 94%, 93%, 92%, 91%, or 90% sequence identity to the parent glucoamylase. Optionally, the variant has more than six amino acids (e.g., 7, 8, 9, 10, 11, 12, 13, 14, 15, 20, 25, 30, 35, 40, 45, 50, 55, or 60) modification compared to the mature form of the parent glucoamylase. The variant possesses the desired pH profile and capability of saccharifying a starch substrate at a pH in the range of 5.0 to 8.0. In some embodiments, the variants may possess other improved properties, such as improved thermostability and improved specificity.

Glucoamylases consist of as many as three distinct structural domains, a catalytic domain of approximately 450 residues that is structurally conserved in all glucoamylases, generally followed by a linker region consisting of between 30 and 80 residues that are connected to a starch binding domain of approximately 100 residues. For example, TrGA has a catalytic domain having the amino acid sequence of SEQ ID NO: 7. The structure of the Trichoderma reesei glucoamylase (TrGA) with all three regions intact was determined to 1.8 Angstrom resolution. See WO 2009/048488 and WO 2009/048487. Using the determined coordinates, the structure was aligned with the coordinates of the catalytic domain of the glucoamylase from Aspergillus awamori strain X100 that was determined previously (Aleshin, A. E., Hoffman, C., Firsov, L. M., and Honzatko, R. B. Refined crystal structures of glucoamylase from Aspergillus awamori var. X100. J. Mol. Biol. 238: 575-591 (1994)). See id. The structure of the catalytic domains of TrGA and Aspergillus awamori glucoamylase overlap very closely, and it is possible to identify equivalent residues based on this structural superposition. See id. It is further believed that all glucoamylases share the basic structure. See id.

Given the well-known structure and function relationship of glucoamylases, glucoamylase variants having altered properties have been successfully created and characterized. The variants may display improved properties as compared to the parent glucoamylases. The improved properties may include, and are not limited to, increased thermostability and increased specific activity. For example, methods for making and characterizing TrGA variants with altered properties have been described in WO 2009/067218. Functional TrGA variants have been identified having one or more specific sequence modifications. Some TrGA variants, for example, have multiple sequence modifications. WO 2009/067218 discloses TrGA variants with six or more amino acid modifications, for example. These TrGA variants show at least as much activity as the parent TrGA, and in many cases show improved properties. It is expected that corresponding residue changes in HgGA and RhGA, for example, will yield variants with glucoamylase activity. The glucoamylase variants useful in the present methods have, at a pH of 6.0 or above, at least about 50% activity relative to the maximum activity.

Production of Glucoamylase

Glucoamylases suitable for the embodiments of the present disclosure may be produced with recombinant DNA technology in various host cells.

In some embodiments, the host cells are selected from bacterial, fungal, plant and yeast cells. The term host cell includes both the cells, progeny of the cells and protoplasts created from the cells that are used to produce a variant glucoamylase according to the disclosure. In some embodiments, the host cells are fungal cells and typically filamentous fungal host cells. The term “filamentous fungi” refers to all filamentous forms of the subdivision Eumycotina (See, Alexopoulos, C. J. (1962), INTRODUCTORY MYCOLOGY, Wiley, New York). These fungi are characterized by a vegetative mycelium with a cell wall composed of chitin, cellulose, and other complex polysaccharides. The filamentous fungi of the present disclosure are morphologically, physiologically, and genetically distinct from yeasts. Vegetative growth by filamentous fungi is by hyphal elongation and carbon catabolism is obligatory aerobic. In the embodiments of the present disclosure, the filamentous fungal parent cell may be a cell of a species of, but not limited to, Trichoderma, (e.g., Trichoderma reesei, the asexual morph of Hypocrea jecorina, previously classified as T. longibrachiatum, Trichoderma viride, Trichoderma koningii, Trichoderma harzianum) (Sheir-Neirs et al., (1984) Appl. Microbiol. Biotechnol 20:46-53; ATCC No. 56765 and ATCC No. 26921); Penicillium sp., Humicola sp. (e.g., H. insolens, H. lanuginosa and H. grisea); Chrysosporium sp. (e.g., C. lucknowense), Gliocladium sp., Aspergillus sp. (e.g., A. oryzae, A. niger, A sojae, A. japonicus, A. nidulans, and A. awamori) (Ward et al., (1993) Appl. Microbiol. Biotechnol. 39:738-743 and Goedegebuur et al., (2002) Genet. 41:89-98), Fusarium sp., (e.g., F. roseum, F. graminum F. cerealis, F. oxysporuim and F. venenatum), Neurospora sp., (N. crassa), Hypocrea sp., Mucor sp., (M. miehei), Rhizopus sp. and Emericella sp. (see also, Innis et al., (1985) Sci. 228:21-26). The term “Trichoderma” or “Trichoderma sp.” or “Trichoderma spp.” refers to any fungal genus previously or currently classified as Trichoderma. In other embodiments, the host cell will be a genetically engineered host cell wherein native genes have been inactivated, for example by deletion in fungal cells. Where it is desired to obtain a fungal host cell having one or more inactivated genes known methods may be used (e.g. methods disclosed in U.S. Pat. Nos. 5,246,853 and 5,475,101, and WO 92/06209). Gene inactivation may be accomplished by complete or partial deletion, by insertional inactivation or by any other means that renders a gene nonfunctional for its intended purpose (such that the gene is prevented from expression of a functional protein). In some embodiments, when the host cell is a Trichoderma cell and particularly a T. reesei host cell, the cbh1, cbh2, egl1 and egl2 genes will be inactivated and/or typically deleted. Typically, Trichoderma reesei host cells having quad-deleted proteins are set forth and described in U.S. Pat. No. 5,847,276 and WO 05/001036. In other embodiments, the host cell is a protease deficient or protease minus strain.

To produce the glucoamylase of the embodiments of the present disclosure with the recombinant DNA technology, a DNA construct comprising nucleic acid encoding the amino acid sequence of the designated glucoamylase can be constructed and transferred into, for example, a Trichoderma reesei host cell. The vector may be any vector which when introduced into a Trichoderma reesei host cell can be integrated into the host cell genome and can be replicated. Reference is made to the Fungal Genetics Stock Center Catalogue of Strains (FGSC, <www.fgsc.net>) for a list of vectors. Additional examples of suitable expression and/or integration vectors are provided in Sambrook et al., (1989) supra, and Ausubel (1987) supra, and van den Hondel et al. (1991) in Bennett and Lasure (Eds.) MORE GENE MANIPULATIONS IN FUNGI, Academic Press pp. 396-428 and U.S. Pat. No. 5,874,276. The nucleic acid encoding the glucoamylase can be operably linked to a suitable promoter, which shows transcriptional activity in Trichoderma reesei host cell. The promoter may be derived from genes encoding proteins either homologous or heterologous to the host cell. Suitable non-limiting examples of promoters include cbh1, cbh2, egl1, egl2. In one embodiment, the promoter may be a native T. reesei promoter. Typically, the promoter can be T. reesei cbh1, which is an inducible promoter and has been deposited in GenBank under Accession No. D86235. An “inducible promoter” may refer to a promoter that is active under environmental or developmental regulation. In another embodiment, the promoter can be one that is heterologous to T. reesei host cell. Other examples of useful promoters include promoters from A. awamori and A. niger glucoamylase genes (see, e.g., Nunberg et al., (1984) Mol. Cell. Biol. 4:2306-2315 and Boel et al., (1984) EMBO J. 3:1581-1585). Also, the promoters of the T. reesei xln1 gene and the cellobiohydrolase 1 gene may be useful (EPA 13f280A1).

In some embodiments, the glucoamylase coding sequence can be operably linked to a signal sequence. The signal sequence may be the native signal peptide of the glucoamylase (residues 1-20 of SEQ ID NO: 2 for HgGA, or residues 1-33 of SEQ ID NO: 5 for TrGA, for example). Alternatively, the signal sequence may have at least 90% or at least 95% sequence identity to the native signal sequence. In additional embodiments, a signal sequence and a promoter sequence comprising a DNA construct or vector to be introduced into the T. reesei host cell are derived from the same source. For example, in some embodiments, the signal sequence can be the cdhl signal sequence that is operably linked to a cdhl promoter.

In some embodiments, the expression vector may also include a termination sequence. In one embodiment, the termination sequence and the promoter sequence can be derived from the same source. In another embodiment, the termination sequence can be homologous to the host cell. A particularly suitable terminator sequence can be cbh1 derived from T. reesei. Other exemplary fungal terminators include the terminator from A. niger or A. awamori glucoamylase gene.

In some embodiments, an expression vector may include a selectable marker. Examples of representative selectable markers include ones that confer antimicrobial resistance (e.g., hygromycin and phleomycin). Nutritional selective markers also find use in the present invention including those markers known in the art as amdS, argB, and pyr4. Markers useful in vector systems for transformation of Trichoderma are known in the art (see, e.g., Finkelstein, chapter 6 in BIOTECHNOLOGY OF FILAMENTOUS FUNGI, Finkelstein et al. Eds. Butterworth-Heinemann, Boston, Mass. (1992), Chap. 6.; and Kinghorn et al. (1992) APPLIED MOLECULAR GENETICS OF FILAMENTOUS FUNGI, Blackie Academic and Professional, Chapman and Hall, London). In a representative embodiment, the selective marker may be the amdS gene, which encodes the enzyme acetamidase, allowing transformed cells to grow on acetamide as a nitrogen source. The use of A. nidulans amdS gene as a selective marker is described for example in Kelley et al., (1985) EMBO J. 4:475-479 and Penttila et al., (1987) Gene 61:155-164.

An expression vector comprising a DNA construct with a polynucleotide encoding the glucoamylase may be any vector which is capable of replicating autonomously in a given fungal host organism or of integrating into the DNA of the host. In some embodiments, the expression vector can be a plasmid. In typical embodiments, two types of expression vectors for obtaining expression of genes are contemplated.

The first expression vector may comprise DNA sequences in which the promoter, glucoamylase-coding region, and terminator all originate from the gene to be expressed. In some embodiments, gene truncation can be obtained by deleting undesired DNA sequences (e.g., DNA encoding unwanted domains) to leave the domain to be expressed under control of its own transcriptional and translational regulatory sequences.

The second type of expression vector may be preassembled and contains sequences needed for high-level transcription and a selectable marker. In some embodiments, the coding region for the glucoamylase gene or part thereof can be inserted into this general-purpose expression vector such that it is under the transcriptional control of the expression construct promoter and terminator sequences. In some embodiments, genes or part thereof may be inserted downstream of a strong promoter, such as the strong cbh1 promoter.

Methods used to ligate the DNA construct comprising a polynucleotide encoding the glucoamylase, a promoter, a terminator and other sequences and to insert them into a suitable vector are well known in the art. Linking can be generally accomplished by ligation at convenient restriction sites. If such sites do not exist, the synthetic oligonucleotide linkers are used in accordance with conventional practice. (see, Sambrook (1989) supra, and Bennett and Lasure, MORE GENE MANIPULATIONS IN FUNGI, Academic Press, San Diego (1991) pp 70-76). Additionally, vectors can be constructed using known recombination techniques (e.g., Invitrogen Life Technologies, Gateway Technology).

Introduction of a DNA construct or vector into a host cell includes techniques such as transformation; electroporation; nuclear microinjection; transduction; transfection, (e.g., lipofection mediated and DEAE-Dextrin mediated transfection); incubation with calcium phosphate DNA precipitate; high velocity bombardment with DNA-coated microprojectiles; and protoplast fusion. General transformation techniques are known in the art (see, e.g., Ausubel et al., (1987), supra, chapter 9; and Sambrook (1989) supra, and Campbell et al., (1989) Curr. Genet. 16:53-56). The expression of heterologous protein in Trichoderma is described in U.S. Pat. Nos. 6,022,725; 6,268,328; Harkki et al. (1991); Enzyme Microb. Technol. 13:227-233; Harkki et al., (1989) Bio Technol. 7:596-603; EP 244,234; EP 215,594; and Nevalainen et al., “The Molecular Biology of Trichoderma and its Application to the Expression of Both Homologous and Heterologous Genes,” in MOLECULAR INDUSTRIAL MYCOLOGY, Eds. Leong and Berka, Marcel Dekker Inc., NY (1992) pp. 129-148).

In some embodiments, genetically stable transformants can be constructed with vector systems whereby the nucleic acid encoding glucoamylase is stably integrated into a host strain chromosome. Transformants are then purified by known techniques.

In one non-limiting example, stable transformants including an amdS marker are distinguished from unstable transformants by their faster growth rate and the formation of circular colonies with a smooth, rather than ragged outline on solid culture medium containing acetamide. Additionally, in some cases a further test of stability can be conducted by growing the transformants on solid non-selective medium (i.e., medium that lacks acetamide), harvesting spores from this culture medium and determining the percentage of these spores which subsequently germinate and grow on selective medium containing acetamide. Alternatively, other methods known in the art may be used to select transformants.

Uptake of DNA into the host Trichoderma sp. strain is dependent upon the calcium ion concentration. Generally, between about 10 mM CaCl₂ and 50 mM CaCl₂ may be used in an uptake solution. Besides the need for the calcium ion in the uptake solution, other compounds generally included are a buffering system such as TE buffer (10 mM Tris, pH 7.4; 1 mM EDTA) or 10 mM MOPS, pH 6.0 buffer (morpholinepropanesulfonic acid) and polyethylene glycol (PEG). It is believed that the polyethylene glycol acts to fuse the cell membranes, thus permitting the contents of the medium to be delivered into the cytoplasm of the Trichoderma sp. strain and the plasmid DNA is transferred to the nucleus. This fusion frequently leaves multiple copies of the plasmid DNA integrated into the host chromosome.

Usually a suspension containing the Trichoderma sp. protoplasts or cells that have been subjected to a permeability treatment at a density of 10⁵ to 10⁷/mL, typically, 2×10⁶/mL are used in transformation. A volume of 100 μL of these protoplasts or cells in an appropriate solution (e.g., 1.2 M sorbitol; 50 mM CaCl₂) are mixed with the desired DNA. Generally, a high concentration of PEG may be added to the uptake solution. From 0.1 to 1 volume of 25% PEG 4000 can be added to the protoplast suspension. It is also typical to add about 0.25 volumes to the protoplast suspension. Additives such as dimethyl sulfoxide, heparin, spermidine, potassium chloride and the like may also be added to the uptake solution and aid in transformation. Similar procedures are available for other fungal host cells. See, e.g., U.S. Pat. Nos. 6,022,725 and 6,268,328.

Generally, the mixture can be then incubated at approximately 0° C. for a period of between 10 to 30 minutes. Additional PEG may then be added to the mixture to further enhance the uptake of the desired gene or DNA sequence. The 25% PEG 4000 can be generally added in volumes of 5 to 15 times the volume of the transformation mixture; however, greater and lesser volumes may be suitable. The 25% PEG 4000 may be typically about 10 times the volume of the transformation mixture. After the PEG is added, the transformation mixture can then be incubated either at room temperature or on ice before the addition of a sorbitol and CaCl₂ solution. The protoplast suspension can then be further added to molten aliquots of a growth medium. This growth medium permits the growth of transformants only.

Generally, cells are cultured in a standard medium containing physiological salts and nutrients (see, e.g., Pourquie, J. et al., BIOCHEMISTRY AND GENETICS OF CELLULOSE DEGRADATION, eds. Aubert, J. P. et al., Academic Press, pp. 7186, 1988 and Ilmen, M. et al., (1997) Appl. Environ. Microbiol. 63:1298-1306). Common commercially prepared media (e.g., Yeast Malt Extract (YM) broth, Luria Bertani (LB) broth and Sabouraud Dextrose (SD) broth also find use in the present embodiments.

Culture-conditions are also standard, e.g., cultures are incubated at approximately 28° C. in appropriate medium in shake cultures or fermentors until desired levels of glucoamylase expression are achieved. After fungal growth has been established, the cells are exposed to conditions effective to cause or permit the expression of the glucoamylase. In cases where the glucoamylase coding sequence is under the control of an inducible promoter, the inducing agent (e.g., a sugar, metal salt or antimicrobial), can be added to the medium at a concentration effective to induce glucoamylase expression.

In general, the glucoamylase produced in cell culture may be secreted into the medium and may be purified or isolated, e.g., by removing unwanted components from the cell culture medium. In some cases, the glucoamylase can be produced in a cellular form, necessitating recovery from a cell lysate. In such cases, the enzyme may be purified from the cells in which it was produced using techniques routinely employed by those of skill in the art. Examples of these techniques include, but are not limited to, affinity chromatography (Tilbeurgh et al., (1984) FEBS Lett. 16: 215), ion-exchange chromatographic methods (Goyal et al., (1991) Biores. Technol. 36: 37; Fliess et al., (1983) Eur. J. Appl. Microbiol. Biotechnol. 17: 314; Bhikhabhai et al, (1984) J. Appl. Biochem. 6: 336; and Ellouz et al., (1987) Chromatography 396: 307), including ion-exchange using materials with high resolution power (Medve et al., (1998) J. Chromatography A 808: 153), hydrophobic interaction chromatography (see, Tomaz and Queiroz, (1999) J. Chromatography A 865: 123; two-phase partitioning (see, Brumbauer, et al., (1999) Bioseparation 7: 287); ethanol precipitation; reverse phase HPLC, chromatography on silica or on a cation-exchange resin such as DEAE, chromatofocusing, SDS-PAGE, ammonium sulfate precipitation, and gel filtration (e.g., Sephadex G-75).

Alpha-Amylases

Alpha-amylases constitute a group of enzymes present in microorganisms and tissues from animals and plants. They are capable of hydrolyzing alpha-1,4-glucosidic bonds of glycogen, starch, related polysaccharides, and some oligosaccharides. Although all alpha-amylases possess the same catalytic function, their amino acid sequences vary greatly. The sequence identity between different amylases can be virtually non-existent, e.g., falling below 25%. Despite considerable amino acid sequence variation, alpha-amylases share a common overall topological scheme that has been identified after the three-dimensional structures of alpha-amylases from different species have been determined. The common three-dimensional structure reveals three domains: (1) a “TIM” barrel known as domain A, (2) a long loop region known as domain B that is inserted within domain A, and (3) a region close to the C-terminus known as domain C that contains a characteristic beta-structure with a Greek-key motif.

“Termamyl-like” alpha-amylases refer to a group of alpha-amylases widely used in the starch-processing industry. The Bacillus licheniformis alpha-amylase having an amino acid sequence of SEQ ID NO: 2 of U.S. Pat. No. 6,440,716 is commercially available as Termamyl®. Termamyl-like alpha-amylases commonly refer to a group of highly homologous alpha-amylases produced by Bacillus spp. Other members of the group include the alpha-amylases from Geobacillus stearothermophilus (previously known as Bacillus stearothermophilus; both names are used interchangeably in the present disclosure) and Bacillus amyloliquefaciens, and those derived from Bacillus sp. NCIB 12289, NCIB 12512, NCIB 12513, and DSM 9375, all of which are described in detail in U.S. Pat. No. 6,440,716 and WO 95/26397.

Although alpha-amylases universally contain the three domains discussed above, the three-dimensional structures of some alpha-amylases, such as AmyE from Bacillus subtilis, differ from Termamyl-like alpha-amylases. These enzymes are collectively referred as non-Termamyl-like alpha-amylases. “AmyE” for the purpose of this disclosure means a naturally occurring alpha-amylase (EC 3.2.1.1; 1, 4-α-D-glucan glucanohydrolase) from Bacillus subtilis. Representative AmyE enzymes and the variants thereof are disclosed in U.S. patent application Ser. Nos. 12/478,266 and 12/478,368, both filed Jun. 4, 2009, and Ser. No. 12/479,427, filed Jun. 5, 2009.

Other commercially available amylases can be used, e.g., TERMAMYL® 120-L, LC and SC SAN SUPER®, SUPRA®, and LIQUEZYME® SC available from Novo Nordisk A/S, FUELZYME® FL from Diversa, and CLARASE® L, SPEZYME® FRED, SPEZYME® ETHYL, GC626, and GZYME® G997 available from Danisco, US, Inc., Genencor Division.

Other Enzymes and Enzyme Combinations

In embodiments of the present disclosure, other enzyme(s) may also be supplemented in starch processing, during saccharification and/or fermentation. These supplementary enzymes may include proteases, pullulanases, isoamylases, cellulases, hemicellulases, xylanases, cyclodextrin glycotransferases, lipases, phytases, laccases, oxidases, esterases, cutinases, xylanases, pullulanases, and/or alpha-glucosidases. See e.g., WO 2009/099783. Skilled artisans in the art are well aware of the methods using the above-listed enzymes.

The glucoamylases disclosed herein can be used in combination with any other enzyme. For example, glucoamylase maybe used in combination with amylases (e.g., alpha-amylases). In one embodiment, saccharification and/or fermentation or the simultaneous saccharification and fermentation (SSF) process use glucoamylase and one or more non-starch polysaccharide hydrolyzing enzymes. These enzymes are capable of hydrolyzing complex carbohydrate polymers such as cellulose, hemicellulose, and pectin. Non-limiting examples include cellulases (e.g., endo and exo-glucanases, beta glucosidase) hemicellulases (e.g., xylanases) and pectinases. In another embodiment, saccharification and/or fermentation or the SSF process use glucoamylase, alpha-amylase and one or more non-starch polysaccharide hydrolyzing enzymes. In another embodiment, saccharification and/or fermentation or the SSF process use glucoamylase with phytases, proteases, isoamylases and pullulanases.

In some embodiments, the saccharification and/or fermentation or the SSF process can use at least two non-starch polysaccharide hydrolyzing enzymes. In some embodiments, the saccharification and/or fermentation or the SSF process can use at least three non-starch polysaccharide hydrolyzing enzymes.

Cellulases are enzyme compositions that hydrolyze cellulose (β-1,4-D-glucan linkages) and/or derivatives thereof, such as phosphoric acid swollen cellulose. Cellulases include the classification of exo-cellobiohydrolases (CBH), endoglucanases (EG) and β-glucosidases (BG) (EC3.2.191, EC3.2.1.4 and EC3.2.1.21). Examples of cellulases include cellulases from Penicillium, Trichoderma, Humicola, Fusarium, Thermomonospora, Cellulomonas, Hypocrea, Clostridium, Thermomonospore, Bacillus, Cellulomonas and Aspergillus. Non-limiting examples of commercially available cellulases sold for feed applications are beta-glucanases such as ROVABIO® (Adisseo), NATUGRAIN® (BASF), MULTIFECT® BGL (Danisco Genencor) and ECONASE® (AB Enzymes). Some commercial cellulases includes ACCELERASE®. The cellulases and endoglucanases described in US20060193897A1 also may be used.

Beta-glucosidases (cellobiase) hydrolyzes cellobiose into individual monosaccharides. Various beta glucanases find use in the invention in combination with phytases. Beta glucanases (endo-cellulase-enzyme classification EC 3.2.1.4) also called endoglucanase I, II, and III, are enzymes that will attack the cellulose fiber to liberate smaller fragments of cellulose which is further attacked by exo-cellulase to liberate glucose. Commercial beta-glucanases useful in the methods of the invention include OPTIMASH® BG and OPTIMASH® TBG (Danisco, US, Inc. Genencor Division).

Hemicellulases are enzymes that break down hemicellulose. Hemicellulose categorizes a wide variety of polysaccharides that are more complex than sugars and less complex than cellulose, that are found in plant walls. In some embodiments, a xylanase find use as a secondary enzyme in the methods of the invention. Any suitable xylanase can be used in the invention. Xylanases (e.g. endo-β-xylanases (E.C. 3.2.1.8), which hydrolyze the xylan backbone chain, can be from bacterial sources (e.g., Bacillus, Streptomyces, Clostridium, Acidothermus, Microtetrapsora or Thermonospora) or from fungal sources (Aspergillus, Trichoderma, Neurospora, Humicola, Penicillium or Fusarium (See, e.g., EP473 545; U.S. Pat. No. 5,612,055; WO 92/06209; and WO 97/20920)). Xylanases useful in the invention include commercial preparations (e.g., MULTIFECT® and FEEDTREAT® Y5 (Danisco Genencor), RONOZYME® WX (Novozymes A/S) and NATUGRAIN WHEAT® (BASF). In some embodiments the xylanase is from Trichoderma reesei or a variant xylanase from Trichoderma reesei, or the inherently thermostable xylanase described in EP1222256B1, as well as other xylanases from Aspergillus niger, Aspergillus kawachii, Aspergillus tubigensis, Bacillus circulans, Bacillus pumilus, Bacillus subtilis, Neocallimastix patriciarum, Penicillium species, Streptomyces lividans, Streptomyces thermoviolaceus, Thermomonospora fusca, Trichoderma harzianum, Trichoderma reesei, and Trichoderma viridae.

Phytases that can be used include those enzymes capable of liberating at least one inorganic phosphate from inositol hexaphosphate. Phytases are grouped according to their preference for a specific position of the phosphate ester group on the phytate molecule at which hydrolysis is initiated, (e.g., as 3-phytases (EC 3.1.3.8) or as 6-phytases (EC 3.1.3.26)). A typical example of phytase is myo-inositol-hexakiphosphate-3-phosphohydrolase. Phytases can be obtained from microorganisms such as fungal and bacterial organisms (e.g. Aspergillus (e.g., A. niger, A. terreus, and A. fumigatus), Myceliophthora (M. thermophila), Talaromyces (T. thermophilus) Trichoderma spp (T. reesei). And Thermomyces (See e.g., WO 99/49740)). Also phytases are available from Penicillium species, (e.g., P. hordei (See e.g., ATCC No. 22053), P. piceum (See e.g., ATCC No. 10519), or P. brevi-compactum (See e.g., ATCC No. 48944) (See, e.g. U.S. Pat. No. 6,475,762). Additional phytases that find use in the invention are available from Peniophora, E. coli, Citrobacter, Enterbacter and Buttiauxella (see e.g., WO2006/043178, filed Oct. 17, 2005). Additional phytases useful in the invention can be obtained commercially (e.g. NATUPHOS® (BASF), RONOZYME® P (Novozymes A/S), PHZYME® (Danisco A/S, Diversa) and FINASE® (AB Enzymes).

Various acid fungal proteases (AFP) can be used as part of the combination as well. Acid fungal proteases include for example, those obtained from Aspergillus, Trichoderma, Mucor and Rhizopus, such as A. niger, A. awamori, A. oryzae and M. miehei. AFP can be derived from heterologous or endogenous protein expression of bacteria, plants and fungi sources. IAFP secreted from strains of Trichoderma can be used. Suitable AFP includes naturally occurring wild-type AFP as well as variant and genetically engineered mutant AFP. Some commercial AFP enzymes useful in the invention include FERMGEN® (Danisco US, Inc, Genencor Division), and FORMASE® 200.

Proteases can also be used with glucoamylase and any other enzyme combination. Any suitable protease can be used. Proteases can be derived from bacterial or fungal sources. Sources of bacterial proteases include proteases from Bacillus (e.g., B. amyloliquefaciens, B. lentus, B. licheniformis, and B. subtilis). Exemplary proteases include, but are not limited to, subtilisin such as a subtilisin obtainable from B. amyloliquefaciens and mutants thereof (U.S. Pat. No. 4,760,025). Suitable commercial protease includes MULTIFECT® P 3000 (Danisco Genencor) and SUMIZYME® FP (Shin Nihon). Sources of suitable fungal proteases include, but are not limited to, Trichoderma, Aspergillus, Humicola and Penicillium, for example.

Debranching enzymes, such as an isoamylase (EC 3.2.1.68) or pullulanase (EC 3.2.1.41), can also be used in combination with the glucoamylases in the saccharification and/or fermentation or SSF processes of the invention. A non-limiting example of a pullulanase that can be used is Promozyme®.

### Starch Processing

### Starch Substrates and Raw Materials

Those of skill in the art are well aware of available methods that may be used to prepare starch substrates for use in the processes disclosed herein. For example, a useful starch substrate may be obtained from tubers, roots, stems, legumes, cereals, or whole grain. More specifically, the granular starch comes from plants that produce high amounts of starch. For example, granular starch may be obtained from corn, wheat, barley, rye, milo, sago, cassava, tapioca, sorghum, rice, peas, bean, banana, or potatoes. Corn contains about 60-68% starch; barley contains about 55-65% starch; millet contains about 75-80% starch; wheat contains about 60-65% starch; and polished rice contains about 70-72% starch. Specifically contemplated starch substrates are cornstarch, wheat starch, and barley starch. The starch from a grain may be ground or whole and includes corn solids, such as kernels, bran and/or cobs. The starch may be highly refined raw starch or feedstock from starch refinery processes. Various starches also are commercially available. For example, cornstarch may be available from Cerestar, Sigma, and Katayama Chemical Industry Co. (Japan); wheat starch may be available from Sigma; sweet potato starch may be available from Wako Pure Chemical Industry Co. (Japan); and potato starch may be available from Nakaari Chemical Pharmaceutical Co. (Japan).

### Milling

The starch substrate can be a crude starch from milled whole grain, which contains non-starch fractions, e.g., germ residues and fibers. Milling may comprise either wet milling or dry milling. In wet milling, whole grain can be soaked in water or dilute acid to separate the grain into its component parts, e.g., starch, protein, germ, oil, kernel fibers. Wet milling efficiently separates the germ and meal (i.e., starch granules and protein) and can be especially suitable for production of syrups. In dry milling, whole kernels are ground into a fine powder and processed without fractionating the grain into its component parts. Dry milled grain thus will comprise significant amounts of non-starch carbohydrate compounds, in addition to starch. Most ethanol comes from dry milling. Alternatively, the starch to be processed may be a highly refined starch quality, for example, at least about 90%, at least about 95%, at least about 97%, or at least about 99.5% pure.

### Gelatinization and Liquefaction

In some embodiments of the invention, gelatinazation and/or liquefaction may be used. As used herein, the term “liquefaction” or “liquefy” means a process by which starch is converted to less viscous and soluble shorter chain dextrins. In some embodiments, this process involves gelatinization of starch simultaneously with or followed by the addition of alpha-amylases. Additional liquefaction-inducing enzymes, e.g., a phytase, optionally may be added. In some embodiments, gelatinization is not used. In other embodiments, a separate liquefaction step is not used. Starches can be converted to shorter chains at the same time that saccharification and/or fermentation is performed. In some embodiments, the starch is being converted directly to glucose. In other embodiments, a separate liquefaction step is used prior to saccharification.

In some embodiments, the starch substrate prepared as described above may be slurried with water. The starch slurry may contain starch as a weight percent of dry solids of about 10-55%, about 20-45%, about 30-45%, about 30-40%, or about 30-35%. In some embodiments, the starch slurry is at least about 5%, at least about 10%, at least about 15%, at least about 20%, at least about 25%, at least about 30%, at least about 35%, at least about 40%, at least about 45%, at least about 50%, or at least about 55%.

To optimize alpha-amylase stability and activity, the pH of the slurry may be adjusted to the optimal pH for the alpha-amylases. Alpha-amylases remaining in the slurry following liquefaction may be deactivated by lowering pH in a subsequent reaction step or by removing calcium from the slurry. The pH of the slurry should be adjusted to a neutral pH (e.g., pH 5.0 to 8.0 and any pH in between this range) when the glucoamylases of the invention are used.

The slurry of starch plus the alpha-amylases may be pumped continuously through a jet cooker, which may be steam heated from about 85° C. to up to about 105° C. Gelatinization occurs very rapidly under these conditions, and the enzymatic activity, combined with the significant shear forces, begins the hydrolysis of the starch substrate. The residence time in the jet cooker can be very brief. The partly gelatinized starch may be passed into a series of holding tubes maintained at about 85-105° C. and held for about 5 min. to complete the gelatinization process. These tanks may contain baffles to discourage back mixing. As used herein, the term “secondary liquefaction” refers the liquefaction step subsequent to primary liquefaction, when the slurry is allowed to cool to room temperature. This cooling step can be about 30 minutes to about 180 minutes, e.g., about 90 minutes to 120 minutes. Milled and liquefied grain is also known as mash.

### Saccharification

Following liquefaction, the mash can be further hydrolyzed through saccharification to produce fermentable sugars that can be readily used in the downstream applications. The saccharification of the present embodiments can be carried out at a pH in the range of 5.0 to 8.0, 5.5 to 7.5, 6.0 to 7.5, 6.5 to 7.5, or 7.0 to 7.5, by using a glucoamylase as described above. In other embodiments, the pH used can be 5.0, 5.25, 5.50, 5.75, 6.0, 6.50, 7.0, 7.50 or 8.0.

In one embodiment, at pH 6.0 or higher, the glucoamylase possesses at least about 50%, about 51%, about 52%, about 53%, about 54%, or about 55% activity relative to its maximum activity at the optimum pH. In another embodiment, for a pH range of 6.0 to 7.5, HgGA can have at least 53% activity relative to its maximum activity. In another embodiment, at pH 6.0, TrGA can have at least 50% activity relative to its maximum activity. In one embodiment, a glucoamylase (e.g. HgGA) has 67% maximal activity at pH 7.0. In another embodiment, a glucoamylase (e.g., TrGA) has 66% maximal activity at pH 5.25.

In one embodiment, the glucoamylase may be dosed at the range of about 0.2 to 2.0 GAU/g dss, about 0.5 to 1.5 GAU/g dss, or 1.0 to 1.5 GAU/g dss. In another embodiment, glucoamylase (e.g., TrGA) can be used at a dose of about 1 GAU/gds starch, 2 GAU/gds starch, 3 GAU/gds starch, 4 GAU/gds starch, or 5 GAU/gds starch. In one embodiment, glucoamylase (e.g., HgGA) can be used at a dose of about 0.25 to 1 GAU/gds starch. In another embodiment, glucoamylase (e.g., HgGA) can be used at a dose of about 0.25 GAU/gds starch, 0.5 GAU/gds starch, 0.75 GAU/gds starch, or 1 GAU/gds starch. The saccharification may be performed at about 30 to about 60° C., or about 40 to about 60° C. In some embodiments, the saccharification occurs at ph 7.0 at 32° C. In other embodiments, the saccharification occurs at ph 6.5 at 58° C.

A full saccharification step may typically range 24 to 96 hours, 24 to 72 hours, or 24 to 48 hours. In some embodiments, saccharification occurs after about 2, 4, 6, 7.7, 8, 110, 14, 16, 18, 20, 22, 23.5, 24, 26, 28, 30, 31.5, 34, 36, 38, 40, 42, 44, 46, or 48 hours. In some embodiments, the saccharification step and fermentation step are combined and the process is referred to as simultaneous saccharification and fermentation (SSF).

It is understood that generally, as time elapses, the enzymes (glucoamylase with or without other enzymes, such as alpha-amylases or non-starch polysaccharide hydrolyzing enzyme) reduces the higher sugars to lower DP sugars (such as DP1). The sugar profile can be varied by using different parameters, such as, but not limited to, starting starch substrate, temperature, amount of glucoamylase, type of glucoamylase, and pH. For example, in one embodiment, at 32 degrees Celsius and pH 7.0, the sugar or oligosaccharide distribution during the saccharification process can be between about 0.36% to about 96.50% DP1, about 3.59% to about 11.80% DP2, about 0.12% to about 7.75%, and/or about 2.26% to about 88.30% for higher sugars for HgGA. In another embodiment, at 32 degrees Celsius and pH 7.0, the sugar distribution during the saccharification process can be between about 0.36% to about 79.19% DP1, between about 3.59% to about 9.92% DP2, about 0.17% to about 9.10% DP3 and/or about 17.15% to about 88.30% for higher sugars for TrGA. Thus, in one embodiment, using HgGA, the DP1 content can reach more than 90% after 24 hours. After 45 hours, the DP1 content can reach more than 96%, while the content of higher sugars can decrease to less than 3%. Using TrGA, more than 70% DP1 can be obtained after 24 hours. After 45 hours, the DP1 content can reach about 80%, while the content of higher sugars can drop to less than 20%.

In another embodiment, at 58 degrees Celsius and pH 6.5, the sugar distribution during the saccharification process can be between about 60.66% to about 93.67% DP1, between about 1.49% to about 8.87% DP2, about 0.33% to about 1.93% DP3 and/or about 4.51% to about 28.17% for higher sugars for HgGA. In other embodiments, at 58 degrees Celsius and pH 6.5, the sugar or oligosaccharide distribution during the saccharification process can be between about 37.08% to about 75.25% DP1, about 5.48% to about 10.19% DP2, about 0.46% to about 5.06%, and/or about 18.37% to about 47.47% for higher sugars for TrGA. Thus, in one embodiment, using HgGA, the DP1 content can reach more than 90% after 24 hours. After 48 hours, the DP1 content can reach more than 93%, while the content of higher sugars can decrease to less than 5%. Using TrGA, more than 70% DP1 can be obtained after 24 hours. After 45 hours, the DP1 content can reach about 75%, while the content of higher sugars can drop to about 18%.

In yet another embodiment, at 58 degrees Celsius and pH 6.5, glucoamylases disclosed herein can be used to saccharify a starch substrate where high sugars (e.g., DP4+) is reduced. In some embodiments, the sugar or oligosaccharide distribution during the saccharification process can be between about 81.10% to about 90.36% DP1, about 1.99% to about 3.96% DP2, about 0.49% to about 0.61% DP3, about 4.48% to about 16.13% DP4+ for TrGA. In other embodiments, the sugar or oligosaccharide distribution during the saccharification process can be between about 93.15% to about 95.33% DP1, about 2.10% to about 3.94% DP2, about 0.53% to about 1.00% DP3, about 0.94% to about 3.76% DP4+ for HgGA.

In yet another embodiment, at 58 degrees Celsius and pH 6.4, the sugar or oligosaccharide distribution during the saccharification process can be between about 93.79% to about 96.9% DP1, about 1.55% to about 3.02% DP2, about 0.2% to about 0.49% DP3 and about 0% to about 3.98% DP4+ for HgGA. In some cases, about 93% solubility and about 96.9% glucose yield can be achieved within 24 hours. Continuous saccharification can result in 99% solubility and about 96.8% glucose after about 48 hours.

In another embodiment, at 58 degrees Celsius and pH 6.4, the sugar or oligosaccharide distribution during the saccharification process can be between about 75.08% to about 96.5% DP1, 1.57% to about 9.16% DP2, 0.67% to about 15.76% DP3+. In some cases, HgGA can maintain a significant amount of glucoamylase activity for about 52 hours at pH6.4 to yield continued production of DP1 products, DP2 products, and increase of percentage of soluble solids. Increased amounts of HgGA can result in increased rates of percentage solubilization and DP1 production.

In some embodiments, the invention can be used to produce DP2 sugars for fermentation by yeast. For example, DP2 sugars can be produced from about 3.59% to about 11.80% DP2, from about 3.59% to about 9.92% DP2, from about 1.49% to about 8.87% DP2, from about 5.48% to about 10.19% DP2, from about 1.99% to about 3.96% DP2, from about 2.10% to about 3.94% DP2, from about 1.55% to about 3.02% DP2, or from about 1.57% to about 9.16% DP2.

### Fermentation

In some embodiments of the present disclosure, the fermentable sugars may be subject to batch or continuous fermentation conditions. A classical batch fermentation is a closed system, wherein the composition of the medium is set at the beginning of the fermentation and is not subject to artificial alterations during the fermentation. Thus, at the beginning of the fermentation the medium may be inoculated with the desired organism(s), e.g., a microorganism engineered to produce isoprene. In this method, fermentation can be permitted to occur without the addition of any components to the system. Typically, a batch fermentation qualifies as a “batch” with respect to the addition of the carbon source and attempts are often made at controlling factors such as pH and oxygen concentration. The metabolite and biomass compositions of the batch system change constantly up to the time the fermentation is stopped. Within batch cultures, cells progress through a static lag phase to a high growth log phase, and finally to a stationary phase where growth rate is diminished or halted. If untreated, cells in the stationary phase eventually die. In general, cells in log phase are responsible for the bulk of production of the end product.

A variation on the standard batch system is the “fed-batch fermentation” system, which may be used in some embodiments of the present disclosure. In this variation of a typical batch system, the substrate can be added in increments as the fermentation progresses. Fed-batch systems are particularly useful when catabolite repression is apt to inhibit the metabolism of the cells and where it is desirable to have limited amounts of substrate in the medium. Measurement of the actual substrate concentration in fed-batch systems may be difficult and is therefore estimated on the basis of the changes of measurable factors such as pH, dissolved oxygen and the partial pressure of waste gases such as CO₂. Both batch and fed-batch fermentations are common and well known in the art.

On the other hand, continuous fermentation is an open system where a defined fermentation medium can be added continuously to a bioreactor and an equal amount of conditioned medium can be removed simultaneously for processing. Continuous fermentation generally maintains the cultures at a constant high density where cells are primarily in log phase growth. Continuous fermentation allows for the modulation of one factor or any number of factors that affect cell growth and/or end product concentration. For example, in one embodiment, a limiting nutrient such as the carbon source or nitrogen source can be maintained at a fixed rate while all other parameters are allowed to moderate. In other systems, a number of factors affecting growth can be altered continuously while the cell concentration, measured by media turbidity, may be kept constant. Continuous systems strive to maintain steady state growth conditions. Thus, cell loss due to medium being drawn off must be balanced against the cell growth rate in the fermentation. Methods of modulating nutrients and growth factors for continuous fermentation processes as well as techniques for maximizing the rate of product formation are well known in the art of industrial microbiology.

In further embodiments, by use of appropriate fermenting microorganisms as known in the art, the fermentation end product may include without limitation alcohol, 1,3-propanediol, succinic acid, lactic acid, amino acids, proteins, functional oligosaccharides, and derivatives thereof. See e.g., WO 2008/086811 (methanol, ethanol, propanol, and butanol fermentation); WO 2003/066816, U.S. Pat. Nos. 5,254,467 and 6,303,352 (1,3-propanediol fermentation); U.S. Pat. Nos. RE 37,393, 6,265,190, and 6,596,521 (succinic acid fermentation); U.S. Pat. No. 5,464,760, WO 2003/095659, Mercier et al., J. Chem. Tech. Biotechnol. 55: 111-121, Zhang and Cheryan, Biotechnol. Lett. 13: 733-738 (1991), Linko and Javanainen, Enzyme Microb. Technol. 19: 118-123 (1996), and Tsai and Moon, Appl. Biochem. Biotechnol. 70-72: 417-428 (1998) (lactic acid fermentation); U.S. Pat. Nos. 7,320,882, 7,332,309, 7,666,634, and Zhang et al., Appl. Microbiol. Biotechnol. 77: 355-366 (2007) (fermentation of various amino acids).

### Cells Capable of Isoprene Production

Microorganisms can be engineered to produce isoprene. Further, other co-products can also be made with the isoprene. The cells can be engineered to contain a heterologous nucleic acid encoding an isoprene synthase polypeptide. Various isoprene synthase, DXP pathway polypeptides (e.g., DXS polypeptides), IDI, MVA pathway polypeptides, hydrogenase, hydrogenase maturation or transcription factor polypeptides and nucleic acids can be used in the compositions and methods for production of starting material. Exemplary nucleic acids, polypeptides and enzymes that can be used are described in WO 2009/076676 and WO 2010/003007, both of which would also include the Appendices listing exemplary nucleic acids and polypeptides for isoprene synthase, DXP pathway, MVA pathway, acetyl-CoA-acetyltransferase, HMG-CoA synthase, hydroxymethylglutaryl-CoA reductase, mevalonate kinase, phosphomevalonate kinase, diphosphomevalonate decarboxylase, isopentenyl phosphate kinases (IPK), isopentenyl-diphosphate Delta-isomerase (IDI) and other polypeptide and nucleic acids that one of skill in the art can use to make cells which produce isoprene.

### Isoprene Synthase

Exemplary isoprene synthase nucleic acids include nucleic acids that encode a polypeptide, fragment of a polypeptide, peptide, or fusion polypeptide that has at least one activity of an isoprene synthase polypeptide. Isoprene synthase polypeptides convert dimethylallyl diphosphate (DMAPP) into isoprene. Exemplary isoprene synthase polypeptides include polypeptides, fragments of polypeptides, peptides, and fusions polypeptides that have at least one activity of an isoprene synthase polypeptide. Exemplary isoprene synthase polypeptides and nucleic acids include naturally-occurring polypeptides and nucleic acids from any of the source organisms described herein. In addition, variants of isoprene synthase which confer additional activity may be used as well.

Standard methods can be used to determine whether a polypeptide has isoprene synthase polypeptide activity by measuring the ability of the polypeptide to convert DMAPP into isoprene in vitro, in a cell extract, or in vivo. Isoprene synthase polypeptide activity in the cell extract can be measured, for example, as described in Silver et al., J. Biol. Chem. 270:13010-13016, 1995. In one embodiment, DMAPP (Sigma) can be evaporated to dryness under a stream of nitrogen and rehydrated to a concentration of 100 mM in 100 mM potassium phosphate buffer pH 8.2 and stored at −20° C. To perform the assay, a solution of 5 μL of 1M MgCl₂, 1 mM (250 μg/ml) DMAPP, 65 μL of Plant Extract Buffer (PEB) (50 mM Tris-HCl, pH 8.0, 20 mM MgCl₂, 5% glycerol, and 2 mM DTT) can be added to 25 μL of cell extract in a 20 ml Headspace vial with a metal screw cap and teflon coated silicon septum (Agilent Technologies) and cultured at 37° C. for 15 minutes with shaking. The reaction can be quenched by adding 200 μL of 250 mM EDTA and quantified by GC/MS.

In some embodiments, the isoprene synthase polypeptide or nucleic acid is from the family Fabaceae, such as the Faboideae subfamily. In some embodiments, the isoprene synthase polypeptide or nucleic acid is a polypeptide or nucleic acid from Pueraria montana (kudzu) (Sharkey et al., Plant Physiology 137: 700-712, 2005), Pueraria lobata, poplar (such as Populus alba, Populus nigra, Populus trichocarpa, or Populus alba x tremula (CAC35696) Miller et al., Planta 213: 483-487, 2001) aspen (such as Populus tremuloides) Silver et al., JBC 270(22): 13010-1316, 1995), or English Oak (Quercus robur) (Zimmer et al., WO 98/02550). Suitable isoprene synthases include, but are not limited to, those identified by Genbank Accession Nos. AY341431, AY316691, AY279379, AJ457070, and AY182241. In some embodiments, the isoprene synthase nucleic acid or polypeptide is a naturally-occurring polypeptide or nucleic acid from poplar. In some embodiments, the isoprene synthase nucleic acid or polypeptide is not a naturally-occurring polypeptide or nucleic acid from poplar.

Types of isoprene synthases which can be used and methods of making microorganisms encoding isoprene synthase are also described in International Patent Application Publication No. WO2009/076676; U.S. Publ. 20100048964, US Publ. 2010/0086978, US Publ. 2010/0167370, US Publ. 2010/0113846, US Publ. 2010/0184178, and US Publ. 2010/0167371; U.S. Publ. 2011/0014672, U.S. Publ. 2010/0196977, and US Publ. 2011/0046422; WO 2004/033646 and WO 96/35796.

### Exemplary DXP Pathway Polypeptides and Nucleic Acids

DXS and IDI polypeptides are part of the DXP pathway for the biosynthesis of isoprene. 1-deoxy-D-xylulose-5-phosphate synthase (DXS) polypeptides convert pyruvate and D-glyceraldehyde-3-phosphate into 1-deoxy-D-xylulose-5-phosphate. While not intending to be bound by any particular theory, it is believed that increasing the amount of DXS polypeptide increases the flow of carbon through the DXP pathway, leading to greater isoprene production.

Exemplary DXS polypeptides include polypeptides, fragments of polypeptides, peptides, and fusions polypeptides that have at least one activity of a DXS polypeptide. Standard methods known to one of skill in the art and as taught the references cited herein can be used to determine whether a polypeptide has DXS polypeptide activity by measuring the ability of the polypeptide to convert pyruvate and D-glyceraldehyde-3-phosphate into 1-deoxy-D-xylulose-5-phosphate in vitro, in a cell extract, or in vivo. Exemplary DXS nucleic acids include nucleic acids that encode a polypeptide, fragment of a polypeptide, peptide, or fusion polypeptide that has at least one activity of a DXS polypeptide. Exemplary DXS polypeptides and nucleic acids include naturally-occurring polypeptides and nucleic acids from any of the source organisms described herein as well as mutant polypeptides and nucleic acids derived from any of the source organisms described herein. Exemplary DXS polypeptides and nucleic acids and methods of measuring DXS activity are described in more detail in International Publication No. WO 2009/076676, U.S. patent application Ser. No. 12/335,071 (US Publ. No. 2009/0203102), WO 2010/003007, US Publ. No. 2010/0048964, WO 2009/132220, and US Publ. No. 2010/0003716.

Exemplary DXP pathways polypeptides include, but are not limited to any of the following polypeptides: DXS polypeptides, DXR polypeptides, MCT polypeptides, CMK polypeptides, MCS polypeptides, HDS polypeptides, HDR polypeptides, and polypeptides (e.g., fusion polypeptides) having an activity of one, two, or more of the DXP pathway polypeptides. In particular, DXP pathway polypeptides include polypeptides, fragments of polypeptides, peptides, and fusions polypeptides that have at least one activity of a DXP pathway polypeptide. Exemplary DXP pathway nucleic acids include nucleic acids that encode a polypeptide, fragment of a polypeptide, peptide, or fusion polypeptide that has at least one activity of a DXP pathway polypeptide. Exemplary DXP pathway polypeptides and nucleic acids include naturally-occurring polypeptides and nucleic acids from any of the source organisms described herein as well as mutant polypeptides and nucleic acids derived from any of the source organisms described herein. Exemplary DXP pathway polypeptides and nucleic acids and methods of measuring DXP pathway polypeptide activity are described in more detail in International Publication No.: WO 2010/148150.

In particular, DXS polypeptides convert pyruvate and D-glyceraldehyde 3-phosphate into 1-deoxy-d-xylulose 5-phosphate (DXP). Standard methods can be used to determine whether a polypeptide has DXS polypeptide activity by measuring the ability of the polypeptide to convert pyruvate and D-glyceraldehyde 3-phosphate in vitro, in a cell extract, or in vivo.

DXR polypeptides convert 1-deoxy-d-xylulose 5-phosphate (DXP) into 2-C-methyl-D-erythritol 4-phosphate (MEP). Standard methods can be used to determine whether a polypeptide has DXR polypeptides activity by measuring the ability of the polypeptide to convert DXP in vitro, in a cell extract, or in vivo.

MCT polypeptides convert 2-C-methyl-D-erythritol 4-phosphate (MEP) into 4-(cytidine 5′-diphospho)-2-methyl-D-erythritol (CDP-ME). Standard methods can be used to determine whether a polypeptide has MCT polypeptides activity by measuring the ability of the polypeptide to convert MEP in vitro, in a cell extract, or in vivo.

CMK polypeptides convert 4-(cytidine 5′-diphospho)-2-C-methyl-D-erythritol (CDP-ME) into 2-phospho-4-(cytidine 5′-diphospho)-2-C-methyl-D-erythritol (CDP-MEP). Standard methods can be used to determine whether a polypeptide has CMK polypeptides activity by measuring the ability of the polypeptide to convert CDP-ME in vitro, in a cell extract, or in vivo.

MCS polypeptides convert 2-phospho-4-(cytidine 5′-diphospho)-2-C-methyl-D-erythritol (CDP-MEP) into 2-C-methyl-D-erythritol 2,4-cyclodiphosphate (ME-CPP or cMEPP). Standard methods can be used to determine whether a polypeptide has MCS polypeptides activity by measuring the ability of the polypeptide to convert CDP-MEP in vitro, in a cell extract, or in vivo.

HDS polypeptides convert 2-C-methyl-D-erythritol 2,4-cyclodiphosphate into (E)-4-hydroxy-3-methylbut-2-en-1-yl diphosphate (HMBPP or HDMAPP). Standard methods can be used to determine whether a polypeptide has HDS polypeptides activity by measuring the ability of the polypeptide to convert ME-CPP in vitro, in a cell extract, or in vivo.

HDR polypeptides convert (E)-4-hydroxy-3-methylbut-2-en-1-yl diphosphate into isopentenyl diphosphate (IPP) and dimethylallyl diphosphate (DMAPP). Standard methods can be used to determine whether a polypeptide has HDR polypeptides activity by measuring the ability of the polypeptide to convert HMBPP in vitro, in a cell extract, or in vivo.

In some embodiments, the DXS or DXP pathway polypeptide is an endogenous polypeptide. In some embodiments, the cells comprise one or more additional copies of an endogenous nucleic acid encoding a DXS or DXP pathway polypeptide. In other embodiments, the DXS or DXP pathway polypeptide is a heterologous polypeptide. In some embodiments, the cells comprise more than one copy of a heterologous nucleic acid encoding an DXS or DXP pathway polypeptide. In any of the embodiments herein, the nucleic acid is operably linked to a promoter (e.g., inducible or constitutive promoter).

### MVA Pathway

In some aspects of the invention, the cells described in any of the compositions or methods described herein comprise a nucleic acid encoding an MVA pathway polypeptide. In some embodiments, the MVA pathway polypeptide is an endogenous polypeptide. In some embodiments, the cells comprise one or more additional copies of an endogenous nucleic acid encoding an MVA pathway polypeptide. In some embodiments, the endogenous nucleic acid encoding an MVA pathway polypeptide operably linked to a constitutive promoter. In some embodiments, the endogenous nucleic acid encoding an MVA pathway polypeptide operably linked to a constitutive promoter. In some embodiments, the endogenous nucleic acid encoding an MVA pathway polypeptide is operably linked to a strong promoter. In a particular embodiment, the cells are engineered to over-express the endogenous MVA pathway polypeptide relative to wild-type cells.

In some embodiments, the MVA pathway polypeptide is a heterologous polypeptide. In some embodiments, the cells comprise more than one copy of a heterologous nucleic acid encoding an MVA pathway polypeptide. In some embodiments, the heterologous nucleic acid encoding an MVA pathway polypeptide is operably linked to a constitutive promoter. In some embodiments, the heterologous nucleic acid encoding an MVA pathway polypeptide is operably linked to a strong promoter.

Exemplary MVA pathway polypeptides include acetyl-CoA acetyltransferase (AA-CoA thiolase) polypeptides, 3-hydroxy-3-methylglutaryl-CoA synthase (HMG-CoA synthase) polypeptides, 3-hydroxy-3-methylglutaryl-CoA reductase (HMG-CoA reductase) polypeptides, mevalonate kinase (MVK) polypeptides, phosphomevalonate kinase (PMK) polypeptides, diphosphomevalonate decarboxylase (MVD) polypeptides, phosphomevalonate decarboxylase (PMDC) polypeptides, isopentenyl phosphate kinase (IPK) polypeptides, IDI polypeptides, and polypeptides (e.g., fusion polypeptides) having an activity of two or more MVA pathway polypeptides. In particular, MVA pathway polypeptides include polypeptides, fragments of polypeptides, peptides, and fusions polypeptides that have at least one activity of an MVA pathway polypeptide. Exemplary MVA pathway nucleic acids include nucleic acids that encode a polypeptide, fragment of a polypeptide, peptide, or fusion polypeptide that has at least one activity of an MVA pathway polypeptide. Exemplary MVA pathway polypeptides and nucleic acids include naturally-occurring polypeptides and nucleic acids from any of the source organisms described herein. In addition, variants of MVA pathway polypeptide that confer the result of better isoprene production can also be used as well.

In some embodiments, feedback resistant mevalonate kinase polypeptides can be used to increase the production of isoprene. As such, the invention provides methods for producing isoprene wherein the host cells further comprise (i) one or more non-modified nucleic acids encoding feedback-resistant mevalonate kinase polypeptides or (ii) one or more additional copies of an endogenous nucleic acid encoding a feedback-resistant mevalonate kinase polypeptide. Non-limiting examples of mevalonate kinase which can be used include: archaeal mevalonate kinase (e.g., from M. mazei, Lactobacillus mevalonate kinase polypeptide, Lactobacillus sakei mevalonate kinase polypeptide, yeast mevalonate kinase polypeptide, Streptococcus mevalonate kinase polypeptide, Streptococcus pneumoniae mevalonate kinase polypeptide, Streptomyces mevalonate kinase polypeptide, and Streptomyces CL190 mevalonate kinase polypeptide.

In another embodiment, aerobes are engineered with isoprene synthase using standard techniques known to one of skill in the art. In another embodiment, anaerobes are engineered with isoprene synthase and one or more MVA pathway polypeptides using standard techniques known to one of skill in the art. In yet another embodiment, either aerobes or anaerobes are engineered with isoprene synthase, one or more MVA pathway polypeptides and/or one or more DXP pathway polypeptides using standard techniques known to one of skill in the art.

Types of MVA pathway polypeptides and/or DXP pathway polypeptides which can be used and methods of making microorganisms (e.g., facultative anaerobes such as E. coli) encoding MVA pathway polypeptides and/or DXP pathway polypeptides are also described in International Patent Application Publication No. WO2009/076676; U.S. Publ. 20100048964, US Publ. 2010/0086978, US Publ. 2010/0167370, US Publ. 2010/0113846, US Publ. 2010/0184178, and US Publ. 2010/0167371; U.S. Publ. 2011/0014672, U.S. Publ. 2010/0196977, and US Publ. 2011/0046422; WO 2004/033646 and WO 96/35796.

One of skill in the art can readily select and/or use suitable promoters to optimize the expression of isoprene synthase or and one or more MVA pathway polypeptides and/or one or more DXP pathway polypeptides in anaerobes. Similarly, one of skill in the art can readily select and/or use suitable vectors (or transfer vehicle) to optimize the expression of isoprene synthase or and one or more MVA pathway polypeptides and/or one or more DXP pathway polypeptides in anaerobes. In some embodiments, the vector contains a selective marker. Examples of selectable markers include, but are not limited to, antibiotic resistance nucleic acids (e.g., kanamycin, ampicillin, carbenicillin, gentamicin, hygromycin, phleomycin, bleomycin, neomycin, or chloramphenicol) and/or nucleic acids that confer a metabolic advantage, such as a nutritional advantage on the host cell. In some embodiments, an isoprene synthase or MVA pathway nucleic acid integrates into a chromosome of the cells without a selective marker.

In some embodiments, the vector is a shuttle vector, which is capable of propagating in two or more different host species. Exemplary shuttle vectors are able to replicate in E. coli and/or Bacillus subtilis and in an obligate anaerobe, such as Clostridium. Upon insertion of an isoprene synthase or MVA pathway nucleic acid into the shuttle vector using techniques well known in the art, the shuttle vector can be introduced into an E. coli host cell for amplification and selection of the vector. The vector can then be isolated and introduced into an obligate anaerobic cell for expression of the isoprene synthase or MVA pathway polypeptide.

### Exemplary IDI Polypeptides and Nucleic Acids

Isopentenyl diphosphate isomerase polypeptides (isopentenyl-diphosphate delta-isomerase or IDI) catalyses the interconversion of isopentenyl diphosphate (IPP) and dimethyl allyl diphosphate (DMAPP) (e.g., converting IPP into DMAPP and/or converting DMAPP into IPP). While not intending to be bound by any particular theory, it is believed that increasing the amount of IDI polypeptide in cells increases the amount (and conversion rate) of IPP that is converted into DMAPP, which in turn is converted into isoprene. Exemplary IDI polypeptides include polypeptides, fragments of polypeptides, peptides, and fusions polypeptides that have at least one activity of an IDI polypeptide. Standard methods can be used to determine whether a polypeptide has IDI polypeptide activity by measuring the ability of the polypeptide to interconvert IPP and DMAPP in vitro, in a cell extract, or in vivo. Exemplary IDI nucleic acids include nucleic acids that encode a polypeptide, fragment of a polypeptide, peptide, or fusion polypeptide that has at least one activity of an IDI polypeptide. Exemplary IDI polypeptides and nucleic acids include naturally-occurring polypeptides and nucleic acids from any of the source organisms described herein as well as mutant polypeptides and nucleic acids derived from any of the source organisms described herein.

### Source Organisms

Isoprene synthase and/or MVA pathway nucleic acids (and their encoded polypeptides) and/or DXP pathway nucleic acids (and their encoded polypeptides) can be obtained from any organism that naturally contains isoprene synthase and/or MVA pathway nucleic acids and/or DXP pathway nucleic acids. As noted above, isoprene is formed naturally by a variety of organisms, such as bacteria, yeast, plants, and animals. Some organisms contain the MVA pathway for producing isoprene. Isoprene synthase nucleic acids can be obtained, e.g., from any organism that contains an isoprene synthase. MVA pathway nucleic acids can be obtained, e.g., from any organism that contains the MVA pathway. DXP pathway nucleic acids can be obtained, e.g., from any organism that contains the DXP pathway.

Exemplary sources for isoprene synthases, MVA pathway polypeptides and/or DXP pathway polypeptides and other polypeptides (including nucleic acids encoding any of the polypeptides described herein) which can be used are also described in International Patent Application Publication No. WO2009/076676; U.S. Publ. 20100048964, US Publ. 2010/0086978, US Publ. 2010/0167370, US Publ. 2010/0113846, US Publ. 2010/0184178, and US Publ. 2010/0167371; U.S. Publ. 2011/0014672, U.S. Publ. 2010/0196977, and US Publ. 2011/0046422; WO 2004/033646 and WO 96/35796.

### Host Cells

Various types of host cells can be used to produce isoprene as part of a bioisoprene composition. In some embodiments, the host cell is a yeast, such as Saccharomyces sp., Schizosaccharomyces sp., Pichia sp., Candida sp. or Y. lipolytica.

In some embodiments, the host cell is a bacterium, such as strains of Bacillus such as B. lichenformis or B. subtilis, strains of Pantoea such as P. citrea, strains of Pseudomonas such as P. alcaligenes, strains of Streptomyces such as S. lividans or S. rubiginosus, strains of Escherichia such as E. coli, strains of Enterobacter, strains of Streptococcus, or strains of Archaea such as Methanosarcina mazei.

As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In some embodiments, the host cell is a gram-positive bacterium. Non-limiting examples include strains of Streptomyces (e.g., S. lividans, S. coelicolor, or S. griseus) and Bacillus. In some embodiments, the source organism is a gram-negative bacterium, such as E. coli or Pseudomonas sp.

In some embodiments, the host cell is a plant, such as a plant from the family Fabaceae, such as the Faboideae subfamily. In some embodiments, the source organism is kudzu, poplar (such as Populus alba x tremula CAC35696), aspen (such as Populus tremuloides), or Quercus robur.

In some embodiments, the host cell is an algae, such as a green algae, red algae, glaucophytes, chlorarachniophytes, euglenids, chromista, or dinoflagellates.

In some embodiments, the host cell is a cyanobacteria, such as cyanobacteria classified into any of the following groups based on morphology: Chroococcales, Pleurocapsales, Oscillatoriales, Nostocales, or Stigonematales.

In some embodiments, the host cell is an anaerobic organisms. An “anaerobe” is an organism that does not require oxygen for growth. An anaerobe can be an obligate anaerobe, a facultative anaerobe, or an aerotolerant organism. Such organisms can be any of the organisms listed above, bacteria, yeast, etc. An “obligate anaerobe” is an anaerobe for which atmospheric levels of oxygen can be lethal. Examples of obligate anaerobes include, but are not limited to, Clostridium, Eurobacterium, Bacteroides, Peptostreptococcus, Butyribacterium, Veillonella, and Actinomyces. In one embodiment, the obligate anaerobes can be any one or combination selected from the group consisting of Clostridium ljungdahlii, Clostridium autoethanogenum, Eurobacterium limosum, Clostridium carboxydivorans, Peptostreptococcus productus, and Butyribacterium methylotrophicum. A “facultative anaerobe” is an anaerobe that is capable of performing aerobic respiration in the presence of oxygen and is capable of performing anaerobic fermentation under oxygen-limited or oxygen-free conditions. Examples of facultative anaerobes include, but are not limited to, Escherichia, Pantoea, yeast, and Yarrowia.

In some embodiments, the host cell is a photosynthetic cell. In other embodiments, the host cell is a non-photosynthetic cell.

### Transformation Methods

Nucleic acids encoding isoprene synthase and/or MVA pathway polypeptides and/or DXP pathway polypeptides can be inserted into any host cell using standard techniques for expression of the encoded isoprene synthase and/or MVA pathway polypeptide. General transformation techniques are known in the art (see, e.g., Current Protocols in Molecular Biology (F. M. Ausubel et al. (eds) Chapter 9, 1987; Sambrook et al., Molecular Cloning: A Laboratory Manual, 2^{nd} ed., Cold Spring Harbor, 1989; and Campbell et al., Curr. Genet. 16:53-56, 1989 or “Handbook on Clostridia” (P. Durre, ed., 2004). For obligate anaerobic host cells, such as Clostridium, electroporation, as described by Davis et al., 2005 and in Examples III and IV, can be used as an effective technique. The introduced nucleic acids may be integrated into chromosomal DNA or maintained as extrachromosomal replicating sequences.

Techniques for producing isoprene in cultures of cells that produce isoprene are described in WO 2009/076676, WO 2010/003007, WO 2010/031079, WO 2010/031062, WO 2010/031077, WO 2010/031068, WO 2010/031076, PCT patent application Ser. No. 09/069,862, US 2009/0203102 A1, and US 2010/0003716 A1. In any case, WO 2009/076676, WO 2010/003007, WO 2010/031079, WO 2010/031062, WO 2010/031077, WO 2010/031068, WO 2010/031076, PCT patent application Ser. No. 09/069,862, US 2009/0203102 A1 and US 2010/0003716 A1 teach compositions and methods for the production of increased amounts of isoprene in cell cultures. U.S. patent application Ser. No. 12/335,071 and US 2009/0203102 A1 further teaches compositions and methods for co-production of isoprene and hydrogen from cultured cells. In particular, these compositions and methods compositions and methods increase the rate of isoprene production and increase the total amount of isoprene that is produced.

As discussed above, the amount of isoprene produced by cells can be greatly increased by introducing a heterologous nucleic acid encoding an isoprene synthase polypeptide (e.g., a plant isoprene synthase polypeptide) into the cells. Isoprene synthase polypeptides convert dimethyl allyl diphosphate (DMAPP) into isoprene.

Additionally, isoprene production by cells that contain a heterologous isoprene synthase nucleic acid can be enhanced by increasing the amount of a 1-deoxy-D-xylulose-5-phosphate synthase (DXS) polypeptide and/or an isopentenyl diphosphate isomerase (IDI) polypeptide expressed by the cells.

Iron-sulfur cluster-interacting redox polypeptide can also be used to increase the activity demonstrated by the DXP pathway polypeptides (such as HDS (GcpE or IspG) or HDR polypeptide (IspH or LytB). While not intending to be bound to a particular theory, the increased expression of one or more endogenous or heterologous iron-sulfur interacting redox nucleic acids or polypeptides improve the rate of formation and the amount of DXP pathway polypeptides containing an iron sulfur cluster (such as HDS or HDR), and/or stabilize DXP pathway polypeptides containing an iron sulfur cluster (such as HDS or HDR). This in turn increases the carbon flux to isoprene synthesis in cells by increasing the synthesis of HMBPP and/or DMAPP and decreasing the cMEPP and HMBPP pools in the DXP pathway.

Additional Host cell Mutations

The invention also contemplates additional host cell mutations that increase carbon flux through the MVA pathway. By increasing the carbon flow, more isoprene can be produced. The recombinant cells as described herein can also be engineered for increased carbon flux towards mevalonate production wherein the activity of one or more enzymes from the group consisting of: (a) citrate synthase, (b) phosphotransacetylase; (c) acetate kinase; (d) lactate dehydrogenase; (e) NADP-dependent malic enzyme, and; (f) pyruvate dehydrogenase is modulated.

### Citrate Synthase Pathway

Citrate synthase catalyzes the condensation of oxaloacetate and acetyl-CoA to form citrate, a metabolite of the Tricarboxylic acid (TCA) cycle (Ner, S. et al. 1983. Biochemistry 22: 5243-5249; Bhayana, V. and Duckworth, H.1984. Biochemistry 23: 2900-2905). In E. coli, this enzyme, encoded by gltA, behaves like a trimer of dimeric subunits. The hexameric form allows the enzyme to be allosterically regulated by NADH. This enzyme has been widely studied (Wiegand, G., and Remington, S. 1986. Annual Rev. Biophysics Biophys. Chem. 15: 97-117; Duckworth et al. 1987. Biochem Soc Symp. 54:83-92; Stockell, D. et al. 2003. J. Biol. Chem. 278: 35435-43; Maurus, R. et al. 2003. Biochemistry. 42:5555-5565). To avoid allosteric inhibition by NADH, replacement by or supplementation with the Bacillus subtilis NADH-insensitive citrate synthase has been considered (Underwood et al. 2002. Appl. Environ. Microbiol. 68:1071-1081; Sanchez et al. 2005. Met. Eng. 7:229-239).

The reaction catalyzed by citrate synthase is directly competing with the thiolase catalyzing the first step of the mevalonate pathway, as they both have acetyl-CoA as a substrate (Hedl et al. 2002. J. Bact. 184:2116-2122). Therefore, one of skill in the art can modulate citrate synthase expression (e.g., decrease enzyme activity) to allow more carbon to flux into the mevalonate pathway, thereby increasing the eventual production of mevalonate and isoprene. Decrease of citrate synthase activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%. In some aspects, the activity of citrate synthase is modulated by decreasing the activity of an endogenous citrate synthase gene. This can be accomplished by chromosomal replacement of an endogenous citrate synthase gene with a transgene encoding an NADH-insensitive citrate synthase or by using a transgene encoding an NADH-insensitive citrate synthase that is derived from Bacillus subtilis. The activity of citrate synthase can also be modulated (e.g., decreased) by replacing the endogenous citrate synthase gene promoter with a synthetic constitutively low expressing promoter. The decrease of the activity of citrate synthase can result in more carbon flux into the mevalonate dependent biosynthetic pathway in comparison to microorganisms that do not have decreased expression of citrate synthase.

Pathways Involving Phosphotransacetylase and/or Acetate Kinase

Phosphotransacetylase (pta) (Shimizu et al. 1969. Biochim. Biophys. Acta 191: 550-558) catalyzes the reversible conversion between acetyl-CoA and acetylphosphate (acetyl-P), while acetate kinase (ackA) (Kakuda, H. et al. 1994. J. Biochem. 11:916-922) uses acetyl-P to form acetate. These genes can be transcribed as an operon in E. coli. Together, they catalyze the dissimilation of acetate, with the release of ATP. Thus, one of skill in the art can increase the amount of available acetyl Co-A by attenuating the activity of phosphotransacetylase gene (e.g., the endogenous phosphotransacetylase gene) and/or an acetate kinase gene (e.g., the endogenous acetate kinase gene). One way of achieving attenuation is by deleting phosphotransacetylase (pta) and/or acetate kinase (ackA). This can be accomplished by replacing one or both genes with a chloramphenicol cassette followed by looping out of the cassette. Acetate is produced by E. coli for a variety of reasons (Wolfe, A. 2005. Microb. Mol. Biol. Rev. 69:12-50). Without being bound by theory, since ackA-pta use acetyl-CoA, deleting those genes might allow carbon not to be diverted into acetate and to increase the yield of mevalonate or isoprene.

In some aspects, the recombinant microorganism produces decreased amounts of acetate in comparison to microorganisms that do not have attenuated endogenous phosphotransacetylase gene and/or endogenous acetate kinase gene expression. Decrease in the amount of acetate produced can be measured by routine assays known to one of skill in the art. The amount of acetate reduction is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The activity of phosphotransacetylase (pta) and/or acetate kinase (ackA) can also be decreased by other molecular manipulation of the enzymes. The decrease of enzyme activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

In some cases, attenuating the activity of the endogenous phosphotransacetylase gene and/or the endogenous acetate kinase gene results in more carbon flux into the mevalonate dependent biosynthetic pathway in comparison to microorganisms that do not have attenuated endogenous phosphotransacetylase gene and/or endogenous acetate kinase gene expression.

### Pathways Involving Lactate Dehydrogenase

In E. coli, D-Lactate is produced from pyruvate through the enzyme lactate dehydrogenase (ldhA) (Bunch, P. et al. 1997. Microbiol. 143:187-195). Production of lactate is accompanied with oxidation of NADH, hence lactate is produced when oxygen is limited and cannot accommodate all the reducing equivalents. Thus, production of lactate could be a source for carbon consumption. As such, to improve carbon flow through to mevalonate and isoprene production, one of skill in the art can modulate the activity of lactate dehydrogenase, such as by decreasing the activity of the enzyme.

Accordingly, in one aspect, the activity of lactate dehydrogenase can be modulated by attenuating the activity of an endogenous lactate dehydrogenase gene. Such attenuation can be achieved by deletion of the endogenous lactate dehydrogenase gene. Other ways of attenuating the activity of lactate dehydrogenase gene known to one of skill in the art may also be used. By manipulating the pathway that involves lactate dehydrogenase, the recombinant microorganism produces decreased amounts of lactate in comparison to microorganisms that do not have attenuated endogenous lactate dehydrogenase gene expression. Decrease in the amount of lactate produced can be measured by routine assays known to one of skill in the art. The amount of lactate reduction is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The activity of lactate dehydrogenase can also be decreased by other molecular manipulations of the enzyme. The decrease of enzyme activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

Accordingly, in some cases, attenuation of the activity of the endogenous lactate dehydrogenase gene results in more carbon flux into the mevalonate dependent biosynthetic pathway in comparison to microorganisms that do not have attenuated endogenous lactate dehydrogenase gene expression.

Pathways Involving Malic Enzyme

Malic enzyme (in E. coli sfcA and maeB) is an anaplerotic enzyme that catalyzes the conversion of malate into pyruvate (using NAD+ or NADP+) by the equation below:

(S)-malate+NAD(P)⁺ [symbol] pyruvate+CO₂+NAD(P)H

Thus, the two substrates of this enzyme are (S)-malate and NAD(P)⁺, whereas its 3 products are pyruvate, CO₂, and NADPH.

Expression of the NADP-dependent malic enzyme (maeB) (Iwikura, M. et al. 1979. J. Biochem. 85: 1355-1365) can help increase mevalonate and isoprene yield by 1) bringing carbon from the TCA cycle back to pyruvate, direct precursor of acetyl-CoA, itself direct precursor of the mevalonate pathway and 2) producing extra NADPH which could be used in the HMG-CoA reductase reaction (Oh, M K et al. (2002) J. Biol. Chem. 277: 13175-13183; Bologna, F. et al. (2007) J. Bact. 189:5937-5946).

As such, more starting substrate (pyruvate or acetyl-CoA) for the downstream production of mevalonate and isoprene can be achieved by modulating, such as increasing, the activity and/or expression of malic enzyme. The NADP-dependent malic enzyme gene can be an endogenous gene. One non-limiting way to accomplish this is by replacing the endogenous NADP-dependent malic enzyme gene promoter with a synthetic constitutively expressing promoter. Another non-limiting way to increase enzyme activity is by using one or more heterologous nucleic acids encoding an NADP-dependent malic enzyme polypeptide. One of skill in the art can monitor the expression of maeB RNA during fermentation or culturing using readily available molecular biology techniques.

Accordingly, in some embodiments, the recombinant microorganism produces increased amounts of pyruvate in comparison to microorganisms that do not have increased expression of an NADP-dependent malic enzyme gene. In some aspects, increasing the activity of an NADP-dependent malic enzyme gene results in more carbon flux into the mevalonate dependent biosynthetic pathway in comparison to microorganisms that do not have increased NADP-dependent malic enzyme gene expression.

Increase in the amount of pyruvate produced can be measured by routine assays known to one of skill in the art. The amount of pyruvate increase can be at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The activity of malic enzyme can also be increased by other molecular manipulations of the enzyme. The increase of enzyme activity can be any amount of increase of specific activity or total activity as compared to when no manipulation has been effectuated. In some instances, the increase of enzyme activity is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

Pathways Involving Pyruvate Dehydrogenase Complex

The pyruvate dehydrogenase complex, which catalyzes the decarboxylation of pyruvate into acetyl-CoA, is composed of the proteins encoded by the genes aceE, aceF and lpdA. Transcription of those genes is regulated by several regulators. Thus, one of skill in the art can increase acetyl-CoA by modulating the activity of the pyruvate dehydrogenase complex. Modulation can be to increase the activity and/or expression (e.g., constant expression) of the pyruvate dehydrogenase complex. This can be accomplished by different ways, for example, by placing a strong constitutive promoter, like PL.6 (aattcatataaaaaacatacagataaccatctgcggtgataaattatctctggcggtgttgacataaataccactggcggtgatactgagcaca tcagcaggacgcactgaccaccatgaaggtg—lambda promoter, GenBank NC_{—}001416), in front of the operon or using one or more synthetic constitutively expressing promoters.

Accordingly, in one aspect, the activity of pyruvate dehydrogenase is modulated by increasing the activity of one or more genes of the pyruvate dehydrogenase complex consisting of (a) pyruvate dehydrogenase (E1), (b) dihydrolipoyl transacetylase, and (c) dihydrolipoyl dehydrogenase. It is understood that any one, two or three of these genes can be manipulated for increasing activity of pyruvate dehydrogenase. In another aspect, the activity of the pyruvate dehydrogenase complex can be modulated by attenuating the activity of an endogenous pyruvate dehydrogenase complex repressor gene, further detailed below. The activity of an endogenous pyruvate dehydrogenase complex repressor can be attenuated by deletion of the endogenous pyruvate dehydrogenase complex repressor gene.

In some cases, one or more genes of the pyruvate dehydrogenase complex are endogenous genes. Another way to increase the activity of the pyruvate dehydrogenase complex is by introducing into the microorganism one or more heterologous nucleic acids encoding one or more polypeptides from the group consisting of (a) pyruvate dehydrogenase (E1), (b) dihydrolipoyl transacetylase, and (c) dihydrolipoyl dehydrogenase.

By using any of these methods, the recombinant microorganism can produce increased amounts of acetyl Co-A in comparison to microorganisms wherein the activity of pyruvate dehydrogenase is not modulated. Modulating the activity of pyruvate dehydrogenase can result in more carbon flux into the mevalonate dependent biosynthetic pathway in comparison to microorganisms that do not have modulated pyruvate dehydrogenase expression.

Combinations of Mutations

It is understood that for any of the enzymes and/or enzyme pathways described herein, molecular manipulations that modulate any combination (two, three, four, five or six) of the enzymes and/or enzyme pathways described herein is expressly contemplated. For ease of the recitation of the combinations, citrate synthase (gltA) is designated as A, phosphotransacetylase (ptaB) is designated as B, acetate kinase (ackA) is designated as C, lactate dehydrogenase (ldhA) is designated as D, malic enzyme (sfcA or maeB) is designated as E, and pyruvate decarboxylase (aceE, aceF, and/or lpdA) is designated as F. As discussed above, aceE, aceF, and/or lpdA enzymes of the pyruvate decarboxylase complex can be used singly, or two of three enzymes, or three of three enzymes for increasing pyruvate decarboxylase activity.

Accordingly, for combinations of any two of the enzymes A-F, non-limiting combinations that can be used are: AB, AC, AD, AE, AF, BC, BD, BE, BF, CD, CE, CF, DE, DF and EF. For combinations of any three of the enzymes A-F, non-limiting combinations that can be used are: ABC, ABD, ABE, ABF, BCD, BCE, BCF, CDE, CDF, DEF, ACD, ACE, ACF, ADE, ADF, AEF, BDE, BDF, BEF, and CEF. For combinations of any four of the enzymes A-F, non-limiting combinations that can be used are: ABCD, ABCE, ABCF, ABDE, ABDF, ABEF, BCDE, BCDF, CDEF, ACDE, ACDF, ACEF, BCEF, BDEF, and ADEF. For combinations of any five of the enzymes A-F, non-limiting combinations that can be used are: ABCDE, ABCDF, ABDEF, BCDEF, ACDEF, and ABCEF. In another aspect, all six enzyme combinations are used: ABCDEF.

Accordingly, the recombinant microorganism as described herein can achieve increased mevalonate production that is increased compared to microorganisms that are not grown under conditions of tri-carboxylic acid (TCA) cycle activity, wherein metabolic carbon flux in the recombinant microorganism is directed towards mevalonate production by modulating the activity of one or more enzymes from the group consisting of (a) citrate synthase, (b) phosphotransacetylase and/or acetate kinase, (c) lactate dehydrogenase, (d) malic enzyme, and (e) pyruvate decarboxylase complex.

Other Regulators and Factors for Increased Production

Other molecular manipulations can be used to increase the flow of carbon towards mevalonate production. One method is to reduce, decrease or eliminate the effects of negative regulators for pathways that feed into the mevalonate pathway. For example, in some cases, the genes aceEF-lpdA are in an operon, with a fourth gene upstream pdhR. pdhR is a negative regulator of the transcription of its operon. In the absence of pyruvate, it binds its target promoter and represses transcription. It also regulates ndh and cyoABCD in the same way (Ogasawara, H. et al. 2007. J. Bact. 189:5534-5541). In one aspect, deletion of pdhR regulator can improve the supply of pyruvate, and hence the production of mevalonate and isoprene.

In other aspects, the introduction of 6-phosphogluconolactonase (PGL) into microorganisms (such as various E. coli strains) which lack PGL can be used to improve production of mevalonate and isoprene. PGL may be introduced using chromosomal integration or extra-chromosomal vehicles, such as plasmids.

### Production of Isoprene Using SSF

Simultaneous saccharification and fermentation can be used to produce isoprene by using cells, which have been engineered to produce isoprene, as an inoculum. Generally, the cells are engineered such they produce a level and/or rate of isoprene at an amount that is commercially desirable, which is detailed below.

Simultaneous saccharification system allows for the production of isoprene more efficiently, measured by total amount of isoprene produced per added amount of starch, by utilizing starch under limited glucose conditions, further detailed below. Isoprene produced by simultaneous saccharification and fermentation at limited glucose conditions also can reduce the volatiles produced under excess glucose conditions and thus has higher purity.

Production of Isoprene within Safe Operating Ranges

The production of isoprene within safe operating levels according to its flammability characteristics simplifies the design and construction of commercial facilities, vastly improves the ability to operate safely, and limits the potential for fires to occur. In particular, the optimal ranges for the production of isoprene are within the safe zone, i.e., the nonflammable range of isoprene concentrations. In one such aspect, the invention features a method for the production of isoprene within the nonflammable range of isoprene concentrations (outside the flammability envelope of isoprene).

Thus, computer modeling and experimental testing were used to determine the flammability limits of isoprene (such as isoprene in the presence of O₂, N₂, CO₂, or any combination of two or more of the foregoing gases) in order to ensure process safety. The flammability envelope is characterized by the lower flammability limit (LFL), the upper flammability limit (UFL), the limiting oxygen concentration (LOC), and the limiting temperature. For a system to be flammable, a minimum amount of fuel (such as isoprene) must be in the presence of a minimum amount of oxidant, typically oxygen. The LFL is the minimum amount of isoprene that must be present to sustain burning, while the UFL is the maximum amount of isoprene that can be present. Above this limit, the mixture is fuel rich and the fraction of oxygen is too low to have a flammable mixture. The LOC indicates the minimum fraction of oxygen that must also be present to have a flammable mixture. The limiting temperature is based on the flash point of isoprene and is that lowest temperature at which combustion of isoprene can propagate. These limits are specific to the concentration of isoprene, type and concentration of oxidant, inerts present in the system, temperature, and pressure of the system. Compositions that fall within the limits of the flammability envelope propagate combustion and require additional safety precautions in both the design and operation of process equipment.

The following conditions were tested using computer simulation and mathematical analysis and experimental testing. If desired, other conditions (such as other temperature, pressure, and permanent gas compositions) may be tested using the methods described herein to determine the LFL, UFL, and LOC concentrations.

### (1) Computer Simulation and Mathematical Analysis

### Test Suite 1:

isoprene: 0 wt %-14 wt %

### O₂: 6 wt %-21 wt %

### N₂: 79 wt %-94 wt %

### Test Suite 2:

isoprene: 0 wt %-14 wt %

### O₂: 6 wt %-21 wt %

### N₂: 79 wt %-94 wt %

Saturated with H₂O

### Test Suite 3:

isoprene: 0 wt %-14 wt %

### O₂: 6 wt %-21 wt %

### N₂: 79 wt %-94 wt %

### CO₂: 5 wt %-30 wt %

### (2) Experimental Testing for Final Determination of Flammability Limits

### Test Suite 1:

isoprene: 0 wt %-14 wt %

### O₂: 6 wt %-21 wt %

### N₂: 79 wt %-94 wt %

### Test Suite 2:

isoprene: 0 wt %-14 wt %

### O₂: 6 wt %-21 wt %

### N₂: 79 wt %-94 wt %

Saturated with H₂O

Simulation software was used to give an estimate of the flammability characteristics of the system for several different testing conditions. CO₂ showed no significant affect on the system's flammability limits. Test suites 1 and 2 were confirmed by experimental testing. The modeling results were in-line with the experimental test results. Only slight variations were found with the addition of water.

The LOC was determined to be 9.5 vol % for an isoprene, O₂, N₂, and CO₂ mixture at 40° C. and 1 atmosphere. The addition of up to 30% CO₂ did not significantly affect the flammability characteristics of an isoprene, O₂, and N₂ mixture. Only slight variations in flammability characteristics were shown between a dry and water saturated isoprene, O₂, and N₂ system. The limiting temperature is about −54° C. Temperatures below about −54° C. are too low to propagate combustion of isoprene.

In some embodiments, the LFL of isoprene ranges from about 1.5 vol. % to about 2.0 vol %, and the UFL of isoprene ranges from about 2.0 vol. % to about 12.0 vol. %, depending on the amount of oxygen in the system. In some embodiments, the LOC is about 9.5 vol % oxygen. In some embodiments, the LFL of isoprene is between about 1.5 vol. % to about 2.0 vol %, the UFL of isoprene is between about 2.0 vol. % to about 12.0 vol. %, and the LOC is about 9.5 vol % oxygen when the temperature is between about 250 C to about 550 C (such as about 400 C) and the pressure is between about 1 atmosphere and 3 atmospheres.

In some embodiments, isoprene is produced in the presence of less than about 9.5 vol % oxygen (that is, below the LOC required to have a flammable mixture of isoprene). In some embodiments in which isoprene is produced in the presence of greater than or about 9.5 vol % oxygen, the isoprene concentration is below the LFL (such as below about 1.5 vol. %). For example, the amount of isoprene can be kept below the LFL by diluting the isoprene composition with an inert gas (e.g., by continuously or periodically adding an inert gas such as nitrogen to keep the isoprene composition below the LFL). In some embodiments in which isoprene is produced in the presence of greater than or about 9.5 vol % oxygen, the isoprene concentration is above the UFL (such as above about 12 vol. %). For example, the amount of isoprene can be kept above the UFL by using a system (such as any of the cell culture systems described herein) that produces isoprene at a concentration above the UFL. If desired, a relatively low level of oxygen can be used so that the UFL is also relatively low. In this case, a lower isoprene concentration is needed to remain above the UFL.

In some embodiments in which isoprene is produced in the presence of greater than or about 9.5 vol % oxygen, the isoprene concentration is within the flammability envelope (such as between the LFL and the UFL). In some embodiments when the isoprene concentration may fall within the flammability envelope, one or more steps are performed to reduce the probability of a fire or explosion. For example, one or more sources of ignition (such as any materials that may generate a spark) can be avoided. In some embodiments, one or more steps are performed to reduce the amount of time that the concentration of isoprene remains within the flammability envelope. In some embodiments, a sensor is used to detect when the concentration of isoprene is close to or within the flammability envelope. If desired, the concentration of isoprene can be measured at one or more time points during the culturing of cells, and the cell culture conditions and/or the amount of inert gas can be adjusted using standard methods if the concentration of isoprene is close to or within the flammability envelope. In particular embodiments, the cell culture conditions (such as fermentation conditions) are adjusted to either decrease the concentration of isoprene below the LFL or increase the concentration of isoprene above the UFL. In some embodiments, the amount of isoprene is kept below the LFL by diluting the isoprene composition with an inert gas (such as by continuously or periodically adding an inert gas to keep the isoprene composition below the LFL).

In some embodiments, the amount of flammable volatiles other than isoprene (such as one or more sugars) is at least about 2, 5, 10, 50, 75, or 100-fold less than the amount of isoprene produced. In some embodiments, the portion of the gas phase other than isoprene gas comprises between about 0% to about 100% (volume) oxygen, such as between about 0% to about 10%, about 10% to about 20%, about 20% to about 30%, about 30% to about 40%, about 40% to about 50%, about 50% to about 60%, about 60% to about 70%, about 70% to about 80%, about 90% to about 90%, or about 90% to about 100% (volume) oxygen. In some embodiments, the portion of the gas phase other than isoprene gas comprises between about 0% to about 99% (volume) nitrogen, such as between about 0% to about 10%, about 10% to about 20%, about 20% to about 30%, about 30% to about 40%, about 40% to about 50%, about 50% to about 60%, about 60% to about 70%, about 70% to about 80%, about 90% to about 90%, or about 90% to about 99% (volume) nitrogen.

In some embodiments, the portion of the gas phase other than isoprene gas comprises between about 1% to about 50% (volume) CO₂, such as between about 1% to about 10%, about 10% to about 20%, about 20% to about 30%, about 30% to about 40%, or about 40% to about 50% (volume) CO₂.

In some embodiments, an isoprene composition also contains ethanol. For example, ethanol may be used for extractive distillation of isoprene, resulting in compositions (such as intermediate product streams) that include both ethanol and isoprene. Desirably, the amount of ethanol is outside the flammability envelope for ethanol. The LOC of ethanol is about 8.7 vol %, and the LFL for ethanol is about 3.3 vol % at standard conditions, such as about 1 atmosphere and about 60° F. (NFPA 69 Standard on Explosion Prevention Systems, 2008 edition, which is hereby incorporated by reference in its entirety, particularly with respect to LOC, LFL, and UFL values). In some embodiments, compositions that include isoprene and ethanol are produced in the presence of less than the LOC required to have a flammable mixture of ethanol (such as less than about 8.7% vol %). In some embodiments in which compositions that include isoprene and ethanol are produced in the presence of greater than or about the LOC required to have a flammable mixture of ethanol, the ethanol concentration is below the LFL (such as less than about 3.3 vol. %).

In various embodiments, the amount of oxidant (such as oxygen) is below the LOC of any fuel in the system (such as isoprene or ethanol). In various embodiments, the amount of oxidant (such as oxygen) is less than about 60, 40, 30, 20, 10, or 5% of the LOC of isoprene or ethanol. In various embodiments, the amount of oxidant (such as oxygen) is less than the LOC of isoprene or ethanol by at least 2, 4, 5, or more absolute percentage points (vol %). In particular embodiments, the amount of oxygen is at least 2 absolute percentage points (vol %) less than the LOC of isoprene or ethanol (such as an oxygen concentration of less than 7.5 vol % when the LOC of isoprene is 9.5 vol %). In various embodiments, the amount of fuel (such as isoprene or ethanol) is less than or about 25, 20, 15, 10, or 5% of the LFL for that fuel.

### Growth Conditions

The cells (e.g., aerobic or anaerobic) of any of the compositions or methods should be grown under conditions that are conducive to optimal production of isoprene. Considerations for optimization include cell culture media, oxygen levels, and conditions favorable for decoupling such that isoprene production is favored over cell growth. For aerobic cells, the cell culture conditions should be used that provide optimal oxygenation for cells to be able to produce isoprene. Consideration should be paid to safety precautions for flammability, such as culturing under oxygen ranges that minimize flammability of the system. See, for example, WO 2010/003007. The production of isoprene within safe operating levels according to its flammability characteristics simplifies the design and construction of commercial facilities, vastly improves the ability to operate safely, and limits the potential for fires to occur. In particular, the optimal ranges for the production of isoprene are within the safe zone, i.e., the nonflammable range of isoprene concentrations. In one such aspect, the invention features a method for the production of isoprene within the nonflammable range of isoprene concentrations (outside the flammability envelope of isoprene).

For anaerobic cells, these cells are capable of replicating and/or producing isoprene in a fermentation system that is substantially free of oxygen. Thus, in one embodiment, anaerobic cells engineered to produce isoprene can use SSF for initial growth. In some embodiments, the fermentation system contains syngas as the carbon and/or energy source. In some embodiments, the anaerobic cells are initially grown in a medium comprising a carbon source other than syngas and then switched to syngas as the carbon source. For the cells that use syngas as a source or energy and/or carbon, the syngas includes at least carbon monoxide and hydrogen. In some embodiments, the syngas further additionally includes one or more of carbon dioxide, water, or nitrogen.

In one aspect, the amount and rate of glucose used for isoprene production can be controlled to maximize the production of isoprene. One of skill in the art should take care to monitor the amount of glucose input since too much glucose can result acetate being produced instead of isoprene. Accordingly, in some embodiments, limited glucose conditions are used. One of skill in the art can control the amount of glucose and glucoamylases' role in regulation of the amount of glucose. The amount of glucoamylase can be optimized to produce glucose at a rate that would keep fermentation glucose limited. Glucoamylase to starch ratio determines that rate of glucose release is more than or equal to rate of glucose utilization by isoprene producing cells, resulting in low or non-detectable glucose conditions. Limited glucose conditions depend on the glucose utilizing microorganism for which glucose concentration range can be 0.2 to 10 g/L. In some embodiments, the glucose concentration range can be at least about 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 g/L. In other embodiments, the glucose concentration range can be at most about 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 g/L.

Renewable resources are used for production of isoprene. Renewable resources refer to resources that are not fossil fuels. Generally, renewable resources are derived from living organisms or recently living organisms that can be replenished as they are consumed. Renewable resources can be replaced by natural ecological cycles or sound management practices. Non-limiting examples include biomass (e.g., switchgrass, hemp, corn, poplar, willow, sorghum, sugarcane), trees, and other plants. Non-limiting examples of renewable resources (or renewable carbon sources) include cheese whey permeate, cornsteep liquor, sugar beet molasses, barley malt, and components from any of the foregoing. Exemplary renewable carbon sources also include glucose, hexose, pentose and xylose present in biomass, such as corn, switchgrass, sugar cane, cell waste of fermentation processes, and protein by-product from the milling of soy, corn, or wheat. In some embodiments, the biomass carbon source is a lignocellulosic, hemicellulosic, or cellulosic material such as, but are not limited to, a grass, wheat, wheat straw, bagasse, sugar cane bagasse, soft wood pulp, corn, corn cob or husk, corn kernel, fiber from corn kernels, corn stover, switch grass, rice hull product, or a by-product from wet or dry milling of grains (e.g., corn, sorghum, rye, triticate, barley, wheat, and/or distillers grains). Exemplary cellulosic materials include wood, paper and pulp waste, herbaceous plants, and fruit pulp. In some embodiments, the carbon source includes any plant part, such as stems, grains, roots, or tubers. In some embodiments, all or part of any of the following plants are used as a carbon source: corn, wheat, rye, sorghum, triticate, rice, millet, barley, cassava, legumes, such as beans and peas, potatoes, sweet potatoes, bananas, sugarcane, and/or tapioca. In some embodiments, the carbon source is a biomass hydrolysate, such as a biomass hydrolysate that includes both xylose and glucose or that includes both sucrose and glucose. As discussed above, the use of simultaneous saccharification and fermentation of any renewable resources can be used for the production of isoprene.

Examples of other fermentation systems and culture conditions which can be used are described in International Patent Application Publication No. WO2009/076676; U.S. Publ. 20100048964, US Publ. 2010/0086978, US Publ. 2010/0167370, US Publ. 2010/0113846, US Publ. 2010/0184178, and US Publ. 2010/0167371; U.S. Publ. 2011/0014672, U.S. Publ. 2010/0196977, and US Publ. 2011/0046422; WO 2004/033646 and WO 96/35796.

### Bioreactors

A variety of different types of reactors can be used for production of isoprene from any renewable resource. There are a large number of different types of fermentation processes that are used commercially. The bioreactor can be designed to optimize the retention time of the cells, the residence time of liquid, and the sparging rate of any gas (e.g., syngas).

In various embodiments, the cells are grown using any known mode of fermentation, such as batch, fed-batch, continuous, or continuous with recycle processes. In some embodiments, a batch method of fermentation is used. Classical batch fermentation is a closed system where the composition of the media is set at the beginning of the fermentation and is not subject to artificial alterations during the fermentation. Thus, at the beginning of the fermentation the cell medium is inoculated with the desired host cells and fermentation is permitted to occur adding nothing to the system. Typically, however, “batch” fermentation is batch with respect to the addition of carbon source and attempts are often made at controlling factors such as pH and oxygen concentration. In batch systems, the metabolite and biomass compositions of the system change constantly until the time the fermentation is stopped. Within batch cultures, cells moderate through a static lag phase to a high growth log phase and finally to a stationary phase where growth rate is diminished or halted. In some embodiments, cells in log phase are responsible for the bulk of the isoprene production. In some embodiments, cells in stationary phase produce isoprene.

In some embodiments, a variation on the standard batch system is used, such as the Fed-Batch system. Fed-Batch fermentation processes comprise a typical batch system with the exception that the carbon source (e.g. syngas, glucose) is added in increments as the fermentation progresses. Fed-Batch systems are useful when catabolite repression is apt to inhibit the metabolism of the cells and where it is desirable to have limited amounts of carbon source in the cell medium. Fed-batch fermentations may be performed with the carbon source (e.g., syngas, glucose, fructose) in a limited or excess amount. Measurement of the actual carbon source concentration in Fed-Batch systems is difficult and is therefore estimated on the basis of the changes of measurable factors such as pH, dissolved oxygen, and the partial pressure of waste gases such as CO₂. Batch and Fed-Batch fermentations are common and well known in the art and examples may be found in Brock, Biotechnology: A Textbook of Industrial Microbiology, Second Edition (1989) Sinauer Associates, Inc.

In some embodiments, continuous fermentation methods are used. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth.

Continuous fermentation allows for the modulation of one factor or any number of factors that affect cell growth or isoprene production. For example, one method maintains a limiting nutrient such as the carbon source or nitrogen level at a fixed rate and allows all other parameters to moderate. In other systems, a number of factors affecting growth can be altered continuously while the cell concentration (e.g., the concentration measured by media turbidity) is kept constant. Continuous systems strive to maintain steady state growth conditions. Thus, the cell loss due to media being drawn off is balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, Biotechnology: A Textbook of Industrial Microbiology, Second Edition (1989) Sinauer Associates, Inc., which is hereby incorporated by reference in its entirety, particularly with respect to cell culture and fermentation conditions.

A variation of the continuous fermentation method is the continuous with recycle method. This system is similar to the continuous bioreactor, with the difference being that cells removed with the liquid content are returned to the bioreactor by means of a cell mass separation device. Cross-filtration units, centrifuges, settling tanks, wood chips, hydrogels, and/or hollow fibers are used for cell mass separation or retention. This process is typically used to increase the productivity of the continuous bioreactor system, and may be particularly useful for anaerobes, which may grow more slowly and in lower concentrations than aerobes.

In one embodiment, a membrane bioreactor can be used for the growth and/or fermentation of the cells described herein, in particular, if the cells are expected to grow slowly. A membrane filter, such as a crossflow filter or a tangential flow filter, can be operated jointly with a liquid fermentation bioreactor that produces isoprene gas. Such a membrane bioreactor can enhance fermentative production of isoprene gas by combining fermentation with recycling of select broth components that would otherwise be discarded. The MBR filters fermentation broth and returns the non-permeating component (filter “retentate”) to the reactor, effectively increasing reactor concentration of cells, cell debris, and other broth solids, while maintaining specific productivity of the cells. This substantially improves titer, total production, and volumetric productivity of isoprene, leading to lower capital and operating costs.

The liquid filtrate (or permeate) is not returned to the reactor and thus provides a beneficial reduction in reactor volume, similar to collecting a broth draw-off. However, unlike a broth draw-off, the collected permeate is a clarified liquid that can be easily sterilized by filtration after storage in an ordinary vessel. Thus, the permeate can be readily reused as a nutrient and/or water recycle source. A permeate, which contains soluble spent medium, may be added to the same or another fermentation to enhance isoprene production.

### Exemplary Production of Bioisoprene Composition

In some embodiments, the cells are cultured in a culture medium under conditions permitting the production of isoprene by the cells in the SSF system with glucoamylase under neutral pH conditions.

By “peak absolute productivity” is meant the maximum absolute amount of isoprene in the off-gas during the culturing of cells for a particular period of time (e.g., the culturing of cells during a particular fermentation run). By “peak absolute productivity time point” is meant the time point during a fermentation run when the absolute amount of isoprene in the off-gas is at a maximum during the culturing of cells for a particular period of time (e.g., the culturing of cells during a particular fermentation run). In some embodiments, the isoprene amount is measured at the peak absolute productivity time point. In some embodiments, the peak absolute productivity for the cells is about any of the isoprene amounts disclosed herein.

By “peak specific productivity” is meant the maximum amount of isoprene produced per cell during the culturing of cells for a particular period of time (e.g., the culturing of cells during a particular fermentation run). By “peak specific productivity time point” is meant the time point during the culturing of cells for a particular period of time (e.g., the culturing of cells during a particular fermentation run) when the amount of isoprene produced per cell is at a maximum. The peak specific productivity is determined by dividing the total productivity by the amount of cells, as determined by optical density at 600 nm (OD₆₀₀). In some embodiments, the isoprene amount is measured at the peak specific productivity time point. In some embodiments, the peak specific productivity for the cells is about any of the isoprene amounts per cell disclosed herein.

By “peak volumetric productivity” is meant the maximum amount of isoprene produced per volume of broth (including the volume of the cells and the cell medium) during the culturing of cells for a particular period of time (e.g., the culturing of cells during a particular fermentation run). By “peak specific volumetric productivity time point” is meant the time point during the culturing of cells for a particular period of time (e.g., the culturing of cells during a particular fermentation run) when the amount of isoprene produced per volume of broth is at a maximum. The peak specific volumetric productivity is determined by dividing the total productivity by the volume of broth and amount of time. In some embodiments, the isoprene amount is measured at the peak specific volumetric productivity time point. In some embodiments, the peak specific volumetric productivity for the cells is about any of the isoprene amounts per volume per time disclosed herein.

By “peak concentration” is meant the maximum amount of isoprene produced during the culturing of cells for a particular period of time (e.g., the culturing of cells during a particular fermentation run). By “peak concentration time point” is meant the time point during the culturing of cells for a particular period of time (e.g., the culturing of cells during a particular fermentation run) when the amount of isoprene produced per cell is at a maximum. In some embodiments, the isoprene amount is measured at the peak concentration time point. In some embodiments, the peak concentration for the cells is about any of the isoprene amounts disclosed herein.

By “average volumetric productivity” is meant the average amount of isoprene produced per volume of broth (including the volume of the cells and the cell medium) during the culturing of cells for a particular period of time (e.g., the culturing of cells during a particular fermentation run). The average volumetric productivity is determined by dividing the total productivity by the volume of broth and amount of time. In some embodiments, the average specific volumetric productivity for the cells is about any of the isoprene amounts per volume per time disclosed herein.

By “cumulative total productivity” is meant the cumulative, total amount of isoprene produced during the culturing of cells for a particular period of time (e.g., the culturing of cells during a particular fermentation run). In some embodiments, the cumulative, total amount of isoprene is measured. In some embodiments, the cumulative total productivity for the cells is about any of the isoprene amounts disclosed herein.

As used herein, “relative detector response” refers to the ratio between the detector response (such as the GC/MS area) for one compound (such as isoprene) to the detector response (such as the GC/MS area) of one or more compounds (such as all C5 hydrocarbons). The detector response may be measured as described herein, such as the GC/MS analysis performed with an Agilent 6890 GC/MS system fitted with an Agilent HP-5MS GC/MS column (30 m×250 μm; 0.25 μm film thickness). If desired, the relative detector response can be converted to a weight percentage using the response factors for each of the compounds. This response factor is a measure of how much signal is generated for a given amount of a particular compound (that is, how sensitive the detector is to a particular compound). This response factor can be used as a correction factor to convert the relative detector response to a weight percentage when the detector has different sensitivities to the compounds being compared. Alternatively, the weight percentage can be approximated by assuming that the response factors are the same for the compounds being compared. Thus, the weight percentage can be assumed to be approximately the same as the relative detector response.

In some embodiments, the cells in culture produce isoprene at greater than or about 3, 4, 5, 6, 7, 8, 9, 10, 11, 12, 13, 14, 15, 16, 17, 18, 19, or 20 g/L (g isoprene/L broth).

In some embodiments, the cells in culture produce isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 12,500, 20,000, 30,000, 40,000, 50,000, 75,000, 100,000, 125,000, 150,000, 188,000, or more nmole of isoprene/gram of cells for the wet weight of the cells/hour (nmole/g_{wcm}/hr). In some embodiments, the amount of isoprene is between about 2 to about 200,000 nmole/g_{wcm}/hr, such as between about 2 to about 100 nmole/g_{wcm}/hr, about 100 to about 500 nmole/g_{wcm}/hr, about 150 to about 500 nmole/g_{wcm}/hr, about 500 to about 1,000 nmole/g_{wcm}/hr, about 1,000 to about 2,000 nmole/g_{wcm}/hr, or about 2,000 to about 5,000 nmole/g_{wcm}/hr, about 5,000 to about 10,000 nmole/g_{wcm}/hr, about 10,000 to about 50,000 nmole/g_{wcm}/hr, about 50,000 to about 100,000 nmole/g_{wcm}/hr, about 100,000 to about 150,000 nmole/g_{wcm}/hr, or about 150,000 to about 200,000 nmole/g_{wcm}/hr. In some embodiments, the amount of isoprene is between about 20 to about 5,000 nmole/g_{wcm}/hr, about 100 to about 5,000 nmole/g_{wcm}/hr, about 200 to about 2,000 nmole/g_{wcm}/hr, about 200 to about 1,000 nmole/g_{wcm}/hr, about 300 to about 1,000 nmole/g_{wcm}/hr, or about 400 to about 1,000 nmole/g_{wcm}/hr, about 1,000 to about 5,000 nmole/g_{wcm}/hr, about 2,000 to about 20,000 nmole/g_{wcm}/hr, about 5,000 to about 50,000 nmole/g_{wcm}/hr, about 10,000 to about 100,000 nmole/g_{wcm}/hr, about 20,000 to about 150,000 nmole/g_{wcm}/hr, or about 20,000 to about 200,000 nmole/g_{wcm}/hr.

The amount of isoprene in units of nmole/g_{wcm}/hr can be measured as disclosed in U.S. Pat. No. 5,849,970, which is hereby incorporated by reference in its entirety, particularly with respect to the measurement of isoprene production. For example, two mL of headspace (e.g., headspace from a culture such as 2 mL of culture cultured in sealed vials at 32° C. with shaking at 200 rpm for approximately 3 hours) are analyzed for isoprene using a standard gas chromatography system, such as a system operated isothermally (85° C.) with an n-octane/porasil C column (Alltech Associates, Inc., Deerfield, Ill.) and coupled to a RGD2 mercuric oxide reduction gas detector (Trace Analytical, Menlo Park, Calif.) (see, for example, Greenberg et al, Atmos. Environ. 27A: 2689-2692, 1993; Silver et al., Plant Physiol. 97:1588-1591, 1991, which are each hereby incorporated by reference in their entireties, particularly with respect to the measurement of isoprene production). The gas chromatography area units are converted to nmol isoprene via a standard isoprene concentration calibration curve. In some embodiments, the value for the grams of cells for the wet weight of the cells is calculated by obtaining the A₆₀₀ value for a sample of the cell culture, and then converting the A₆₀₀ value to grams of cells based on a calibration curve of wet weights for cell cultures with a known A₆₀₀ value. In some embodiments, the grams of the cells is estimated by assuming that one liter of broth (including cell medium and cells) with an A₆₀₀ value of 1 has a wet cell weight of 1 gram. The value is also divided by the number of hours the culture has been incubating for, such as three hours.

### Systems for Producing Isoprene

The invention also provides systems for producing isoprene. In one aspect, the system includes (i) a bioreactor within which saccharification and fermentation are performed at about pH 5.0 to 8.0; (ii) a host cell comprising a heterologous nucleic acid encoding an isoprene synthase polypeptide; (iii) a glucoamylase that possesses at least 50% activity at pH 6.0 or above relative to its maximum activity, wherein the glucoamylase is selected from the group consisting of a parent Humicola grisea glucoamylase (HgGA) comprising SEQ ID NO: 3, a parent Trichoderma reesei glucoamylase (TrGA) comprising SEQ ID NO: 6, a parent Rhizopus p. glucoamylase (RhGA) comprising SEQ ID NO: 9, and a variant thereof, and wherein the variant has at least 99% sequence identity to the parent glucoamylase.

Components of the system are described herein. Various combinations of these system components are expressly contemplated within the scope of the invention.

### Recovery

Optionally, isoprene is recovered from the off-gas of the culture system. Methods and apparatus for the purification of a bioisoprene composition from fermentor off-gas which can be used are described in WO/2011/075534.

A bioisoprene composition from a fermentor off-gas may contain bioisoprene with volatile impurities and bio-byproduct impurities. In some embodiments, a bioisoprene composition from a fermentor off-gas is purified using a method comprising: (a) contacting the fermentor off-gas with a solvent in a first column to form: an isoprene-rich solution comprising the solvent, a major portion of the isoprene and a major portion of the bio-byproduct impurity; and a vapor comprising a major portion of the volatile impurity; (b) transferring the isoprene-rich solution from the first column to a second column; and (c) stripping isoprene from the isoprene-rich solution in the second column to form: an isoprene-lean solution comprising a major portion of the bio-byproduct impurity; and a purified isoprene composition.

The above enumerated list are only examples and one skilled in the art will be aware of a number of fermenting microorganisms that may be appropriately used to obtain a desired end product.

### Simultaneous Saccharification and Fermentation (SSF)

During SSF, the hydrolyzing enzymes are added along with the end product producer, commonly a microorganism. Enzymes release lower molecule sugars, i.e., fermentable sugars DP1-3, from the starch substrate, while the microorganism simultaneously uses the fermentable sugars for growth and production of the end product. Typically, fermentation conditions are selected that provide an optimal pH and temperature for promoting the best growth kinetics of the producer host cell strain and catalytic conditions for the enzymes produced by the culture. See e.g., Doran et al., Biotechnol. Progress 9: 533-538 (1993). Table 1 presents exemplary fermentation microorganism and their optimal pH for fermentation. Because the glucoamylases disclosed herein possess significant activity at a neutral pH and an elevated temperature, they would be useful in the SSF for those microorganisms having an optimal fermenting pH in the range of 5.5 to 7.5.

TABLE 1
Exemplary fermentation organisms and their optimal pH.

| | | Optimal pH of the |
|---|---|---|
| End products | Fermentation Organisms | fermentation |
| Lysine and salts | Corynebacterium glutamicum | 6.8-7.0 |
| thereof | Bacillus lacterosprous | 7.0-7.2 |
| | Methylophilotrophus | 7 |
| Lactic Acid | Lactobacillus amylophilus | 6.0-6.5 |
| | Bacillus coagulans | 6.4-6.6 |
| | Bacillus thermoamylovorans | 5.0-6.5 |
| | Bacillus smithii | 5.0-6.5 |
| | Geobacillus stearothermophilus | 5.0-6.5 |
| Monosodium | Corynebacterium pekinense | 7 |
| Glutamate (MSG) | Corynebacterium crenatum | 7 |
| | Brevibacterium tianjinese | 7 |
| | Corynebacterium glutamicum | 7.0-7.2 |
| | HU7251 | |
| | Arthrobacter sp | 7 |
| Succinic acid | Escherichia coli | 6.0-7.5 |
| 1,3-Propanediol | Escherichia coli | 6.5-7.5 |
| 2-Keto-gulonic acid | Escherichia coli | 5.0-6.0 |
| Isoprene | Escherichia coli | 6-8 |

In further embodiments, by use of appropriate fermenting microorganisms as known in the art to produce the desired end product, those of skill in the art are well capable of adjusting the SSF conditions, e.g., temperature, nutrient composition, light conditions, oxygen availability, etc.

### EXAMPLES

### Methods Used in the Examples

The following materials, assays, and methods were used in the examples provided below:

### HPLC Method to Measure Saccharide Composition

The composition of the reaction products of oligosaccharides was measured by a HPLC system (Beckman System Gold 32 Karat Fullerton, Calif.). The system, maintained at 50° C., was equipped with a Rezex 8 u8% H Monosaccharides column and a refractive index (R1) detector (ERC-7515A, Anspec Company, Inc.). Diluted sulfuric acid (0.01 N) was applied as the mobile phase at a flow rate of 0.6 ml/min. 20 μl of 4.0% solution of the reaction mixture was injected onto the column. The column separates saccharides based on their molecular weights. The distribution of saccharides and the amount of each saccharide were determined from previously run standards.

### Determination of Glucoamylase Activity Units (GAU)

Glucoamylase activity units (GAU) were determined based on the activity of a glucoamylase enzyme to catalyze the hydrolysis of p-nitrophenyl-alpha-D-glucopyranoside (PNPG) to glucose and p-nitrophenol. At an alkaline pH, p-nitrophenol forms a yellow color that is measured spectrophotometrically at 405 nm. The amount of p-nitrophenol released correlates with the glucoamylase activity.

### Protein Concentration Determination

The protein concentration in a sample was determined using the Bradford QuickStart™ Dye Reagent (Bio-Rad, California, USA). For example, a 10 μL sample of the enzyme was combined with 200 μL Bradford QuickStart™ Dye Reagent. After thorough mixing, the reaction mixture was incubated for at least 10 minutes at room temperature. Air bubbles were removed and the optical density (OD) was measured at 595 nm. The protein concentration was then calculated using a standard curve generated from known amounts of bovine serum albumin.

### Purification of HgGA for Characterization Studies

The material concentrated by ultrafiltration (UFC) was desalted/buffer-exchanged using a BioRad DP-10 desalting column and 25 mM Tris pH 8.0. 100 mg of total protein was applied to a Pharmacia Hi Prep 16/10 S Sepharose FF column, which was equilibrated with the above buffer at 5 ml/min. Glucoamylase was eluted with a 4-column volume (CV) gradient buffer containing 0-200 mM NaCl. Multiple runs were performed and the purest fractions, as determined via SDS-PAGE/coomassie blue staining analysis, were pooled and concentrated using VivaSpin 10K MWCO 25 ml spin tubes. The final material was passed over a Novagen HisBind 900 chromatography cartridge that had been washed with 250 mM EDTA and rinsed with above buffer. 2 ml of final material was obtained, having a protein concentration of 103.6 mg/ml, and a glucoamylase activity of 166.1 GAU/ml (determined by a PNPG based assay). Specific activities were determined using a standardized method using p-nitrophenyl-alpha-D-glucopyranoside (PNPG) as a substrate and reported in GAU units.

### Determination of Glucose Concentration

Glucose concentration in a saccharification reaction mixture was determined with the ABTS assay. Samples or glucose standards in 5 μL were placed in wells of a 96-well microtiter plate (MTP). Reactions were initiated with the addition of 95 μL of the reactant containing 2.74 mg/ml 2,2′-Azino-bis(3-ethylbenzothiazoline-6-sulfonic acid) diammonium salt (ABTS) (Sigma P1888), 0.1 U/ml horseradish peroxidase type VI (Sigma P8375), and 1 U/ml glucose oxidase (Sigma G7141). OD_{405 nm} was immediately monitored at a 9-second interval for 300 seconds using a Spectramax plate reader. Because the rate of OD₄₀₅ₙₘ increase is proportional to the glucose concentration, the sample's glucose concentration was determined by comparing with the glucose standard, and was reported as mg/ml.

### Example 1

### Comparison of the pH and Activity Profiles of Various Glucoamylases at 32° C.

The pH and activity profiles of glucoamylases (GAs) from Humicola grisea (HgGA), Trichoderma reesei (TrGA), Aspergillus niger (AnGA) and Talaromyces emersonii (TeGA) were determined at 32° C. As the substrate, 8% potato starch (Sigma Cat. No. 52630) was solubilized by heating. A series of citrate/phosphate buffers at 0.25 or 0.5 pH increments, ranging from pH 2.0 to 8.0, were prepared. Purified enzymes were diluted to 0.1 or 0.02 GAU/ml in water (TeGA was dosed at 0.2 GAU/ml). HgGA, TrGA, AnGA, and TeGA were dosed at 0.0125, 0.0076, 0.0109, and 0.0055 mg/ml, respectively. 10 μL buffer of various pH was placed in 0.2 ml PCR tube strips (AB Gene, Cat. No. AB-0451, 800-445-2812) with 15 μL of diluted enzyme. The reactions were initiated by the addition of 25 μL soluble potato starch. The reactions were incubated on a PCR type thermocycler heating block for exactly ten minutes, then terminated by the addition of 10 μL 0.5 M NaOH. The glucose released in the reaction was determined using the ABTS assay, and the glucoamylase activities were determined. The pH and activity profiles are presented in Table and FIG. 1 as the percentage of the maximum activity for each glucoamylase.

TABLE 2
pH profiles of HgGA, TrGA, AnGA, and TeGA at 32° C. The values
represent % of the maximum activity for each enzyme.

| pH | HgGA | TrGA | AnGA | TeGA |
|---|---|---|---|---|
| 2.00 | 45 | 56 | 91 | 93 |
| 2.50 | 54 | 67 | 91 | 97 |
| 2.75 | 60 | 72 | | 100 |
| 3.00 | 63 | 81 | 98 | 98 |
| 3.25 | 71 | 91 | 100 | 95 |
| 3.50 | 77 | 99 | 99 | 88 |
| 3.75 | 84 | 100 | 96 | 79 |
| 4.00 | 93 | | 84 | 64 |
| 4.25 | 100 | 95 | 78 | 51 |
| 4.50 | 84 | | 55 | 34 |
| 4.75 | 44 | | 46 | 30 |
| 5.00 | 40 | | 45 | 29 |
| 5.25 | 42 | 66 | 43 | 27 |
| 5.50 | 46 | | 41 | 23 |
| 5.75 | 48 | 58 | 39 | 21 |
| 6.00 | 53 | 51 | 35 | 17 |
| 6.50 | 62 | 38 | 27 | 11 |
| 7.00 | 67 | 22 | 17 | 5 |
| 7.50 | 58 | 10 | 7 | 2 |
| 8.00 | 39 | 4 | 3 | 1 |

As shown in Table 2 and FIG. 1, both TeGA and AnGA exhibited significantly reduced activity in the pH range of 6.0 to 8.0. At a pH 5.0 or above, TeGA retained no more than 29% activity relative to its maximum activity. At a pH 6.0 or above, TeGA retained no more than 17% activity relative to its maximum activity. Similarly, at a pH 6.0 of 6.0 or above, AnGA displayed no more than 35% activity relative to its maximum activity. In the pH range of 6.0 to 7.5, HgGA retained at least 53% activity relative to its maximum activity. At pH 6.0, TrGA also displayed at least 50% activity relative to its maximum activity. The above observation shows that both HgGA and TrGA are suitable for producing fermentable sugars at a neutral pH range (as described herein for neutral pH glucoamylases) under fermentation conditions.

### Example 2

### Comparison of Hydrolysis of Solubilized Starch at 32° C., pH 7.0

The ability of various glucoamylases to hydrolyze solubilized starch substrate (liquefact) at a neutral pH was compared. Corn starch was liquefied by following a conventional high-temperature jet cooking process using CLEARFLOW™ AA to a liquefact of DE 12-15. Saccharification of the liquefact (25% DS) was carried out using TrGA, HgGA, and AnGA at 1.0 GAU/g ds at 32° C., pH 7.0. Samples were withdrawn at different time intervals during the saccharification and subject to HPLC analysis. The composition of the oligosaccharides is presented in Table 3.

TABLE 3
Composition of oligosaccharides in saccharification.

% Sugars, pH 7.0, 32° C.

| | | | | | Higher |
|---|---|---|---|---|---|
| GA | Time (hr) | DP1 | DP2 | DP3 | Sugars |
| HgGA | 0 | 0.36 | 3.59 | 7.75 | 88.30 |
| | 2 | 51.10 | 10.20 | 6.87 | 31.85 |
| | 5.25 | 64.90 | 11.80 | 0.13 | 23.13 |
| | 21.25 | 89.30 | 1.10 | 0.30 | 9.34 |
| | 25.25 | 91.20 | 0.98 | 0.23 | 7.61 |
| | 29.25 | 92.60 | 0.90 | 0.31 | 6.12 |
| | 45.25 | 96.50 | 1.15 | 0.12 | 2.26 |
| TrGA | 0 | 0.36 | 3.59 | 7.75 | 88.30 |
| | 2 | 38.06 | 7.49 | 9.10 | 45.35 |
| | 5.25 | 47.17 | 9.92 | 6.13 | 36.78 |
| | 21.25 | 69.43 | 8.33 | 0.17 | 22.07 |
| | 25.25 | 71.69 | 7.14 | 0.17 | 21.01 |
| | 29.25 | 73.57 | 6.16 | 0.18 | 20.09 |
| | 45.25 | 79.19 | 3.45 | 0.20 | 17.15 |
| AnGA | 0 | 0.36 | 3.59 | 7.75 | 88.30 |
| | 2 | 14.12 | 4.57 | 8.88 | 72.43 |
| | 5.25 | 28.38 | 8.01 | 10.30 | 53.31 |
| | 21.25 | 58.97 | 11.49 | 0.28 | 29.26 |
| | 25.25 | 60.94 | 10.53 | 0.28 | 28.25 |
| | 29.25 | 62.82 | 9.54 | 0.23 | 27.41 |
| | 45.25 | 74.14 | 4.08 | 0.24 | 21.54 |

Using HgGA, the DP1 content reached more than 90% after 24 hrs. After 45 hours, the DP1 content reached more than 96%, while the content of higher sugars decreased to less than 3%. Using TrGA, more than 70% DP1 was obtained after 24 hours. After 45 hours, the DP1 content reaches about 80%, while the content of higher sugars dropped to less than 20%. For AnGA, less than 75% of DP1 was obtained after 45 hours, while higher sugars remained more than 20%. The data in Table 3 indicate that both HgGA and TrGA are more effective than AnGA to hydrolyze solubilized starch to glucose, at a neutral pH.

### Example 3

### Comparison of Hydrolysis of Liquefied Starch at 58° C., pH 6.5

Corn starch liquefact (˜9.1DE) obtained by SPEZYME® FRED (Danisco US Inc., Genencor Division) treatment was adjusted to pH 6.5 with NaOH and equilibrated at a 58° C. water bath. AnGA (OPTIDEX™ L-400, Danisco US Inc., Genencor Division), TrGA, and HgGA were added at 0.5 GAU/g ds to each flask containing corn starch liquefact. Saccharification was carried out up to 48 hours with periodical sampling for HPLC analysis. 0.5 mL enzyme-deactivated sample was diluted with 4.5 ml of RO water. The diluted sample was then filtered through 0.45 μm Whatman filters and subject to HPLC analysis. The HPLC analysis was conducted as described in Methods used in the Examples. The composition of the oligosaccharides is presented in Table 4.

TABLE 4
Composition of oligosaccharides in saccharification.

Percent Sugar Composition

| | Hour | % DP1 | % DP2 | % DP3 | % HS |
|---|---|---|---|---|---|
| Liquefact | 0 | 0.49 | 3.02 | 5.52 | 90.98 |
| HgGA | 2 | 60.66 | 8.87 | 1.93 | 28.17 |
| | 4 | 69.92 | 7.43 | 0.69 | 21.75 |
| | 6 | 75.96 | 5.80 | 0.38 | 17.85 |
| | 7.7 | 77.56 | 5.15 | 0.47 | 16.35 |
| | 14 | 84.31 | 2.96 | 0.42 | 11.57 |
| | 23.5 | 88.70 | 2.20 | 0.43 | 8.67 |
| | 31.5 | 90.01 | 1.87 | 0.40 | 6.90 |
| | 48 | 93.67 | 1.49 | 0.33 | 4.51 |
| TrGA | 2 | 37.08 | 10.19 | 5.06 | 47.47 |
| | 4 | 49.25 | 12.12 | 2.12 | 36.42 |
| | 6 | 55.30 | 12.16 | 1.09 | 31.10 |
| | 7.7 | 58.06 | 11.74 | 0.76 | 29.12 |
| | 14 | 63.83 | 9.96 | 0.46 | 25.28 |
| | 23.5 | 68.52 | 8.18 | 0.53 | 22.77 |
| | 31.5 | 70.35 | 7.24 | 0.54 | 21.32 |
| | 48 | 75.25 | 5.48 | 0.50 | 18.37 |
| AnGA | 2 | 41.33 | 11.83 | 4.40 | 42.20 |
| | 4 | 50.08 | 12.95 | 1.60 | 35.04 |
| | 6 | 53.32 | 12.70 | 0.83 | 33.16 |
| | 7.7 | 54.80 | 12.41 | 0.62 | 31.91 |
| | 14 | 58.85 | 11.20 | 0.40 | 29.15 |
| | 23.5 | 61.70 | 10.44 | 0.46 | 27.41 |
| | 31.5 | 62.34 | 10.11 | 0.50 | 26.58 |
| | 48 | 64.23 | 9.83 | 0.59 | 25.01 |

Using HgGA, the DP1 content reached more than 90% after 24 hrs. After 48 hours, the DP1 content reached more than 93%, while the content of higher sugars decreased to less than 5%. Using TrGA, more than 70% DP1 was obtained after 24 hours. After 45 hours, the DP1 content reaches about 75%, while the content of higher sugars dropped to about 18%. For AnGA, less than 65% of DP1 was obtained after 45 hours, while higher sugars remained more than 25%. The data in Table 4 indicate that both HgGA and TrGA are more effective than AnGA, at a neutral pH and 58° C., to hydrolyze solubilized starch to glucose. This observation is consistent with data presented in Table 3, where saccharification was performed at 32° C.

### Example 4

### Comparison of High Sugars (DP4+) Reduction at 58° C., pH 6.5

Various concentrations of AnGA, TrGA, and HgGA were used to saccharify a starch substrate at 58° C., pH 6.5, and the reduction of high sugars (DP4+) was compared. The starch substrate was a 25% cornstarch liquefact, which was liquefied by SPEZYME® FRED (Danisco US Inc., Genencor Division). Glucoamylases were added as shown in Table 5, from 0.25 GAU/gds to 10.0 GAU/gds. The saccharification reaction was conducted at 58° C., pH 6.5. Samples were withdrawn at various time points and the sugar composition was determined by HPLC analysis. The composition of the oligosaccharides is presented in Table 5 and FIG. 2.

TABLE 5
Composition of oligosaccharides in saccharification.

| | | Percent Sugar Composition at 48 hr | | | |
|---|---|---|---|---|---|
| Glucoamylase | GAU/gds starch | DP1 | DP2 | DP3 | DP4+ |
| AnGA | 1 | 64.25 | 5.10 | 0.00 | 30.65 |
| | 2.5 | 73.36 | 1.74 | 0.41 | 24.49 |
| | 5 | 81.26 | 1.05 | 0.46 | 17.22 |
| | 7.5 | 85.53 | 1.48 | 0.44 | 12.13 |
| | 10 | 89.32 | 2.03 | 0.42 | 8.22 |
| TrGA | 1 | 81.10 | 2.28 | 0.49 | 16.13 |
| | 2 | 86.65 | 1.99 | 0.49 | 10.87 |
| | 3 | 90.36 | 2.86 | 0.49 | 8.30 |
| | 4 | 90.48 | 3.17 | 0.52 | 5.83 |
| | 5 | 90.95 | 3.96 | 0.61 | 4.48 |
| HgGA | 0.25 | 93.15 | 2.10 | 1.00 | 3.76 |
| | 0.5 | 95.33 | 2.58 | 0.64 | 1.45 |
| | 0.75 | 95.08 | 3.36 | 0.53 | 1.02 |
| | 1 | 94.57 | 3.94 | 0.56 | 0.94 |

The results presented in Table 5 and FIG. 2 indicated that AnGA resulted in more than 8% of higher sugars (DP4+), at 58° C., pH 6.5, even at a high dosage of glucoamylase, 10.0 GAU/gds. In contrast, lower than 5% of higher sugars (DP4+) was observed for 5 GAU/gds TrGA. HgGA resulted in the lowest levels of higher sugars (DP4+). For example, at 0.5 GAU/gds HgGA, the saccharification mixture contained less than 1.5% of higher sugars (DP4+), which is comparable to the resulted obtained under the current industrial high glucose processing conditions (pH 4.5, 60° C.) using AnGA.

### Example 5

### Continuous Production of Glucose from Granular Cassava Starch by HgGA at a Neutral pH

The capability of HgGA to convert granular unmodified cassava starch to glucose and short chain glucose polymers at a neutral pH was further characterized. A 27% dry substance aqueous slurry of cassava starch was first adjusted to pH 6.4 with sodium carbonate. SPEZYME™ Alpha (Danisco US Inc., Genencor Division) was added at 2 AAU/g ds, and HgGA was added at 1 GAU/g ds. The reaction was carried out for 48 hours at 58° C. with continuous stirring. At selected time intervals, samples of the slurry were removed. The removed sample was added to a 2.5 ml micro-centrifuge tube and centrifuged for 4 minutes at 13,000 rpm. Refractive index (R1) of the supernatant was determined at 30° C. The remaining supernatant was filtered through a 13 mm syringe filter with a 0.45 μm GHP membrane into a 2.5 ml micro-centrifuge tube and boiled for 10 minutes to terminate the amylase activity. 0.5 mL enzyme-deactivated sample was diluted with 4.5 ml of RO water. The diluted sample was then filtered through 0.45 μm Whatman filters and subject to HPLC analysis. The HPLC analysis was conducted as described in Methods used in the Examples.

The total dry substance was determined by taking about 1 ml of the starch slurry into a 2.5 ml spin tube, adding 1 drop of SPEZYME® FRED (Danisco US Inc., Genencor Division) from a micro dispo-pipette, and boiling 10 minutes. Refractive index at 30° C. was determined. The dry substance of the supernatant and the whole sample (total) was determined using appropriate DE tables. The CRA 95 DE Table was used for the supernatant and corrected for consumption of water of hydrolysis. % soluble was calculated as: 100×(the dry substance of the supernatant)/(the total dry substance). The composition of the oligosaccharides is presented in Table 6.

TABLE 6
Saccharide distribution for HgGA-mediated saccharification of cassava
granular starch.

Saccharide Distribution

| Hrs | DP1 | DP2 | DP3 | DP4+ | Soluble % |
|---|---|---|---|---|---|
| 2.50 | 93.799 | 1.726 | 0.499 | 3.976 | 56.20 |
| 7.50 | 96.166 | 1.551 | 0.480 | 1.802 | 78.80 |
| 12.00 | 96.731 | 1.639 | 0.411 | 1.220 | 85.10 |
| 23.50 | 96.928 | 2.204 | 0.326 | 0.541 | 92.80 |
| 48.00 | 96.772 | 3.023 | 0.205 | 0.000 | 99.00 |

As shown in Table 6, the reaction achieved about 93% solubility and yielded about 96.9% glucose within 24 hours. Continuation of saccharification resulted in 99% solubility and about 96.8% glucose after 48 hours.

### Example 6

### Continuous Production of Glucose from Granular Cornstarch by HgGA at a Neutral pH

Corn granular starch was used to characterize HgGA. The experiments were carried out using 32% ds corn granular starch. Water (64.44 g) and starch (35.56 g; at 90% ds) were mixed and the pH of the slurry was increased to 6.4. The starch slurry was placed in a water bath maintained at 58° C. and enzymes were added. The enzymes included SPEZYME™ Alpha (Danisco US Inc., Genencor Division) and HgGA. The starch slurry was maintained at 58° C. for 48 hrs and samples were drawn at 3, 6, 10, 24, 32, and 52 hrs to analyze the % soluble and saccharide profile. The results are presented in Table 7.

TABLE 7
Saccharide distribution for HgGA-mediated
saccharification of corn granular starch

| HgGA | Alpha-amylase | | | | | |
|---|---|---|---|---|---|---|
| (GAU/g ds) | (AAU/g ds) | hour | % Soluble | DP1 | DP2 | DP3+ |
| 1 | 2 | 3 | 56.82 | 94.74 | 1.57 | 3.69 |
| | | 6 | 69.45 | 95.52 | 1.76 | 2.61 |
| | | 10 | 75.96 | 96.50 | 1.79 | 1.43 |
| | | 24 | 91.50 | 95.72 | 2.79 | 0.93 |
| | | 32 | 92.71 | 95.50 | 3.08 | 0.86 |
| | | 52 | 99.66 | 93.94 | 4.42 | 0.67 |
| 0.75 | 2 | 3 | 53.35 | 92.74 | 2.00 | 5.25 |
| | | 6 | 65.87 | 94.69 | 1.77 | 3.43 |
| | | 10 | 73.11 | 95.80 | 1.73 | 2.12 |
| | | 24 | 89.09 | 95.70 | 2.53 | 1.59 |
| | | 32 | 91.01 | 95.75 | 2.64 | 1.01 |
| | | 52 | 98.65 | 95.44 | 3.44 | 1.12 |
| 0.5 | 2 | 3 | 49.06 | 88.36 | 3.36 | 8.29 |
| | | 6 | 61.98 | 92.48 | 2.18 | 5.35 |
| | | 10 | 68.18 | 94.08 | 1.90 | 3.67 |
| | | 24 | 84.14 | 95.56 | 2.03 | 2.23 |
| | | 32 | 87.90 | 95.49 | 2.25 | 2.11 |
| | | 52 | 95.17 | 95.30 | 2.81 | 1.12 |
| 0.25 | 2 | 3 | 44.01 | 75.08 | 9.16 | 15.76 |
| | | 6 | 53.92 | 84.31 | 5.25 | 10.45 |
| | | 10 | 60.97 | 88.25 | 3.72 | 7.81 |
| | | 24 | 76.63 | 93.11 | 2.25 | 4.48 |
| | | 32 | 80.00 | 93.66 | 2.17 | 4.05 |
| | | 52 | 88.37 | 94.55 | 2.31 | 2.89 |

As shown in Table 7, HgGA maintains a significant amount of glucoamylase activity for 52 hrs at pH 6.4, evidenced by the continued production of DP1 and DP2, as well as the continued increase of % soluble solids. The data also suggest that the rates of DP1 production and % solubilization of granular starch depend on the amount of HgGA. An increased amount of HgGA resulted in increased rates of % solubilization and DP1 production.

### Example 7

### Characterization of Granular Starch Hydrolysis by HgGA and SPEZYME™ Alpha at a Neutral pH by Scanning Electron Microscopy

Granular starch from corn, wheat, and cassava was treated with HgGA and SPEZYME™ Alpha. A 28% dry substance aqueous slurry of granular starch was first adjusted to pH 6.4 with sodium carbonate. SPEZYME™ Alpha (Danisco US Inc., Genencor Division) was added at 2 AAU/g ds, and HgGA was added at 1 GAU/g ds. Treatment was carried out at 58° C. with continuous stirring. Samples of the slurry were removed at various time points and subject to scanning electron microscopy (SEM). Slurry samples were laid on SEM sample stubs using double-sided carbon tape. Excess sample was removed by gently dusting the mounted sample with compressed air. Mounted samples were sputter coated with gold (15 nm) for 2 min at 25 mV, using an Emitech K550 Sputter Coater (Squorum Technologies). The scanning electron micrographs are presented in FIG. 3. Before treatment, starch surface was smooth and homogenous. Upon HgGA and SPEZYME™ Alpha treatment, the surface morphology of the granules changed over time. The enzyme blend first created small dimples (0.2-0.5 μm in diameter) on the surface of the starch granules. Quantity and size of the dimples increased over time. At a late stage of the treatment, for example, 48 hours for cassava granular starch, empty shells were spotted. Micrographs of empty shells indicated a complete digestion of the interior of the granule. The mechanism of enzymatic action appears to be starch granule surface peeling. Once the surface has been weakened by external peeling, the amylases penetrate and hydrolyze the interior of the granule (i.e., amylolysis) leaving hollowed out shells.

### Example 8

### Isoprene Production by Fermentation

### 8.1. Materials and Methods

Medium Recipe (per liter fermentation medium): K₂HPO₄ 7.5 g, MgSO₄.7H₂O 2 g, citric acid monohydrate 2 g, ferric ammonium citrate 0.3 g, yeast extract 0.5 g, 1000× Modified Trace Metal Solution 1 ml. All of these components were dissolved in 60 mL DI H₂O to form “Component A.” The following various starch substrates (each contained about 270 g starch) were prepared:

1) Granular cornstarch (270 g) was added to 705 ml DI H₂O and incubated at 34° C. for 30 minutes with agitation. The temperature was then increased to 60° C. and held for an additional 12 hours;
2) Granular endosperm 329 g (82% starch g/g) was added to 646 ml H₂O and incubated at 34° C. for 30 minutes with agitation. The temperature was then increased to 60° C. and held for an additional 12 hours;
3) Granular ground corn 397 g (68% starch g/g) was added to 646 ml H₂O and incubated at 34° C. for 30 minutes with agitation. The temperature was then increased to 60° C. and held for an additional 12 hours;
4) 758 g liquefact corn starch (35.6% dry solids);
5) 950 g liquefact endosperm (28.4% starch g/g and 41.3% dry solids); and
6) 950 g liquefact ground corn (28.4% starch g/g, and 39.7% dry solids). For substrates 1), 2) and 3), a slurry was treated at 60° C. for 12 hours. Component A was heat sterilized (123° C. for 20 minutes) and allowed to cool to 25° C. Both medium solutions were then considered sterile and combined. For substrates 4), 5), and 6), a substrate was mixed with Component A, and the mixture was heat sterilized (123° C. for 20 minutes) and allowed to cool to 25° C.

Subsequently, the pH was adjusted to 7.0 with ammonium hydroxide (28%) and q.s. to volume. Mercury Vitamin Solution (8 mL) and antibiotics were added after solution had been cooled to 34° C.

1000× Modified Trace Metal Solution (per liter): Citric Acid.H₂O 40 g, MnSO₄.H₂O 30 g, NaCl 10 g, FeSO₄.7H₂O 1 g, CoCl₂.6H₂O 1 g, ZnSO₄.7H₂O 1 g, CuSO₄.5H₂O 100 mg, H₃BO₃ 100 mg, NaMoO₄.2H₂O 100 mg. Each component was dissolved one at a time in DI H₂O, pH was adjusted to 3.0 with HCl or NaOH, and then the solution was q.s. to volume and filter sterilized with a 0.22 micron filter.

Mercury Vitamin Solution (per liter): Thiamine hydrochloride 1.0 g, D-(+)-biotin 1.0 g, nicotinic acid 1.0 g, D-pantothenic acid 4.8 g, pyridoxine hydrochloride 4.0 g. Each component was dissolved one at a time in DI H₂O, pH was adjusted to 3.0 with HCl or NaOH, and then the solution was q.s. to volume and filter sterilized with 0.22 micron filter.

The fermentation was performed in a 1.7-L bioreactor with E. coli BL21 cell strain MD09-317: t pgl FRT-PL.2-mKKDyI, pCLUpper (pMCM82) (Spec50), pTrcAlba(MEA)mMVK (pDW34) (Carb50). Further information may be found in references cited herein. The experiment was carried out to monitor isoprene formation from the desired starch substrate at the desired fermentation pH 6.5 and temperature 34° C. A frozen vial of the E. coli strain was thawed and inoculated into tryptone-yeast extract medium. After the inoculum grew to optical density 1.0, measured at 550 nm (OD₅₅₀), 40 mL was used to inoculate a 1.7-L bioreactor and bring the initial tank volume to 0.7 L.

8.2. Isoprene Production by Simultaneous Saccharification and Fermentation (SSF) from Various Starch Substrates with the Combination of the Trichoderma reesei Glucoamylase and an Alpha-Amylase

Starch hydrolysis was initiated at cell inoculation (time zero) by adding 8 GAU/L Trichoderma reesei glucoamylase (TrGA) and 404 AAU/L of SPEZYME™ Alpha (Danisco US Inc., Genencor Division). Additional enzymes were added in amounts shown in Table 8 in order to obtain a starch hydrolysis rate that roughly matched the glucose consumption rate of the cells.

TABLE 8
Amount of enzymes added to the bioreactor over time

| | |
|---|---|
| Amount added | Cumulative amount added |

| | TrGA | | TrGA | |
|---|---|---|---|---|
| Time | GAU/L | Spezyme Alpha | GAU/L | Spezyme Alpha |
| hr | broth | AAU/L broth | broth | AAU/L broth |
| 0.0 | 8 | 404 | 8 | 404 |
| 4.1 | 8 | 404 | 16 | 808 |
| 7.0 | 67 | 3381 | 83 | 4189 |
| 11.3 | 132 | 6677 | 215 | 10866 |
| 12.5 | 213 | 10726 | 428 | 21593 |
| 16.7 | 210 | 10596 | 638 | 32188 |

At various time points of the SSF, samples were taken out and subjected to analysis. Similar results were obtained for the variety of starch substrates used. Representative data are presented in FIGS. 4-7.

Accumulated glucose levels in the fermentor broth over time are shown in FIG. 4. Induction was achieved by adding isopropyl-beta-D-1-thiogalactopyranoside (IPTG). The IPTG concentration was brought to 107 μM when the carbon dioxide evolution rate (CER) reached 25 mmol/L/hr. The IPTG concentration was raised to 202 μM when CER reached 175 mmol/L/hr. The isoprene level in the off gas from the bioreactor was determined using a PerkinElmer iScan mass spectrometer. The isoprene titer increased over the course of the fermentation to a maximum value of 7.6 g/L at 20 hrs (FIG. 5). The total amount of isoprene produced during the 20-hour fermentation was 6.0 g. The metabolic activity profile, as measured by the CER, is shown in FIG. 6. Carbon dioxide evolution rate (CER)=[24.851*(airflow slpm/offgas N2%)*supply N2% *offgas CO2%]/(Fermentor kgs/Broth density)

24.851=(60 min/h*1000 mmol/mol)/(100%*24.14 liters/mol)

24.14 liters is how much volume an ideal gas occupies at 1 atm and 21.1 C.
8.3. Isoprene Production by Simultaneous Saccharification and Fermentation (SSF) from Granular Starch with the Humicola grisea Glucoamylase (HgGA)

Granular cornstarch was prepared as described in Example 8.1. to be use for isoprene production by fermentation. Starch hydrolysis was initiated at cell inoculation (time zero) by adding 2 GAU/L broth of HgGA. Additional enzyme was added by continuous feeding in amounts shown in Table 9 in order to obtain a starch hydrolysis rate that roughly matched the glucose consumption rate of the cells. HgGA was diluted in either 36% glucose or water in order to feed.

TABLE 9
Amount of HgGA added to the bioreactor over time.

| | | Cumulative amount |
|---|---|---|
| | Amount added | added |
| Time | H-GA | H-GA |
| hr | GAU/L broth | GAU/L broth |
| 0.0 | 2 | 2 |
| 4.4 | 0 | 2 |
| 8.0 | 0 | 2 |
| 12.0 | 587 | 589 |
| 16.0 | 1383 | 1972 |
| 20.0 | 1353 | 3325 |
| 24.0 | 1961 | 5286 |
| 28.0 | 9638 | 14924 |
| 32.8 | 21963 | 36887 |

At various time points of the SSF, samples were removed and subject to analysis. Accumulated glucose levels in the fermentor broth over time are shown in FIG. 8. Induction was achieved by adding isopropyl-beta-D-1-thiogalactopyranoside (IPTG). The IPTG concentration was brought to 117 μM when the carbon dioxide evolution rate (CER) reached 25 mmol/L/hr. The IPTG concentration was raised to 224 μM when CER reached 175 mmol/L/hr. The isoprene level in the off gas from the bioreactor was determined using a PerkinElmer iScan mass spectrometer. The isoprene titer increased over the course of the fermentation to a maximum value of 5.2 g/L at 35 hrs (FIG. 9). The total amount of isoprene produced during the 35-hour fermentation was 3.4 g. The metabolic activity profile, as measured by the CER, is shown in FIG. 10. The time course of the ratio of isoprene to carbon dioxide in the gas stream exiting the bioreactor, an indicator of product yield, is shown in FIG. 11. It was observed that both the TrGA+AA or H-GA fermentations reached the same peak instantaneous mol isoprene/mol carbon dioxide ratio (roughly 0.08; ratio correlates with instantaneous carbon yield) as a typical glucose fed-batch fermentation. The similarity of these values despite the different conditions indicates that cells produce isoprene in a comparable manner to the traditional process where glucose is fed to the fermentor. More experimentation was performed to elucidate any possible differences between the use of TrGA+AA or H-GA for the stated application, though it was shown that similar amounts of enzymatic activity units were added over the course of the fermentations. No significant differences between the use of TrGA+AA or H-GA were noted in the current data set.

Without being bound by theory, it appears that the TrGA+AA or H-GA activity is inactivated by some component in the fermentation broth, resulting in the need for continued addition of enzyme to the fermentation to produce glucose for cell utilization/isoprene formation. It was also noted that the fermentation broth dissolved oxygen level was lower than the glucose fed-batch fermentation as a result of the higher viscosity caused by the granular starch substrates. The low dissolved oxygen levels are not anticipated to be observed in fermentations utilizing the liquefact substrates.

### SEQUENCE LISTING

A Sequence Listing, comprising SEQ ID NOs: 1-9, is provided herein and is incorporated herein by reference in its entirety.

SEQ ID NO: 1: genomic sequence coding the full-length Humicola grisea
glucoamylase; putative introns are in bold; corresponds to SEQ ID NO.
1 of U.S. Pat. No. 7,262,041

| | |
|---|---|
| atgcatacct tctccaagct cctcgtcctg ggctctgccg tccagtctgc cctcgggcgg | 60 |
| cctcacggct cttcgcgtct ccaggaacgc gctgccgttg ataccttcat caacaccgag | 120 |
| aagcccatcg catggaacaa gctgctcgcc aacatcggcc ctaacggcaa agccgctccc | 180 |
| ggtgccgccg ccggcgttgt gattgccagc ccttccagga cggaccctcc ttgtacgtgg | 240 |
| tggcatggaa tggacccaag agactggttt tagatgaaag agagtttctg ctaaccgcca | 300 |
| cacccagact tcttcacctg gacccgcgat gccgccctgg tcctcaccgg catcatcgag | 360 |
| tcccttggcc acaactacaa caccaccctg cagaccgtca tccagaacta cgtcgcgtcg | 420 |
| caggccaagc tgcagcaggt ctcgaacccc tcgggaacct tcgccgacgg ctcgggtctc | 480 |
| ggtgaggcca agttcaatgt cgacctcact gccttcactg gcgaatgggg tcgccctcag | 540 |
| agggacggcc cgcccctgcg cgccatcgct ctcatccagt acgccaagtg gctgatcgcc | 600 |
| aacggctaca agagcacggc caagagcgtc gtctggcccg tcgtcaagaa cgatctcgcc | 660 |
| tacacggccc agtactggaa cgagaccggc ttcgatctct gggaggaggt ccccggcagc | 720 |
| tcgttcttta ccatcgccag ctctcacagg ggtgagtcat ttattgttca gtgttttctc | 780 |
| attgaataat taccggaatg ccactgacgc caaacagctc tgactgaggg tgcttacctc | 840 |
| gccgctcagc tcgacaccga gtgccgcgcc tgcacgaccg tcgcccctca ggttctgtgc | 900 |
| ttccagcagg ccttctggaa ctccaagggc aactatgtcg tctccaacag taagatccct | 960 |
| acaccaacaa aaaaaatcga aaaggaacgt tagctgaccc ttctagtcaa cggcggcgag | 1020 |
| tatcgctccg gcaaggacgc caactcgatc ctggcgtcca tccacaactt cgaccctgag | 1080 |
| gccggctgcg acaacctgac cttccagccc tgcagcgagc gcgccctggc caaccacaag | 1140 |
| gcctatgtcg actcgttccg caacctctac gccatcaaca agggcatcgc ccagggcaag | 1200 |
| gccgttgccg tcggccgcta ctcggaggat gtctactaca acggcaaccc gtggtacctg | 1260 |
| gccaactttg ccgccgccga gcagctctac gacgccatct acgtgtggaa caagcagggc | 1320 |
| tccatcaccg tgacctcggt ctccctgccc ttcttccgcg accttgtctc gtcggtcagc | 1380 |
| accggcacct actccaagag cagctcgacc ttcaccaaca tcgtcaacgc cgtcaaggcc | 1440 |
| tacgccgacg gcttcatcga ggtggcggcc aagtacaccc cgtccaacgg cgcgctcgcc | 1500 |
| gagcagtacg accgcaacac gggcaagccc gactcggccg ccgacctgac gtggtcgtac | 1560 |
| tcggccttcc tctcggccat cgaccgccgc gcgggtctcg tccccccgag ctggcgggcc | 1620 |
| agcgtggcca agagccagct gccgtccacc tgctcgcgca tcgaggtcgc cggcacctac | 1680 |
| gtcgccgcca cgagcacctc gttcccgtcc aagcagaccc cgaacccctc cgcggcgccc | 1740 |
| tccccgtccc cctacccgac cgcctgcgcg gacgctagcg aggtgtacgt caccttcaac | 1800 |
| gagcgcgtgt cgaccgcgtg gggcgagacc atcaaggtgg tgggcaacgt gccggcgctg | 1860 |
| gggaactggg acacgtccaa ggcggtgacc ctgtcggcca gcgggtacaa gtcgaatgat | 1920 |
| cccctctgga gcatcacggt gcccatcaag gcgacgggct cggccgtgca gtacaagtat | 1980 |
| atcaaggtcg gcaccaacgg gaagattact tgggagtcgg accccaacag gagcattacc | 2040 |
| ctgcagacgg cgtcgtctgc gggcaagtgc gccgcgcaga cggtgaatga ttcgtggcgt | 2100 |
| taa | 2103 |

```
SEQ ID NO: 2: amino acid sequence of full-length Humicola grisea
glucoamylase; the nat\grave{ }ive signal sequence is in bold; corresponds
to SEQ ID NO. 2 of U.S. Pat. No. 7,262,041
    1 MHTFSKLLVL GSAVQSALGR PHGSSRLQER AAVDTFINTE KPIAWNKLLA NIGPNGKAAP
  61 GAAAGVVIAS PSRTDPPYFF TWTRDAALVL TGIIESLGHN YNTTLQTVIQ NYVASQAKLQ
121 QVSNPSGTFA DGSGLGEAKF NVDLTAFTGE WGRPQRDGPP LRAIALIQYA KWLIANGYKS
181 TAKSVVWPVV KNDLAYTAQY WNETGFDLWE EVPGSSFFTI ASSHRALTEG AYLAAQLDTE
241 CRACTTVAPQ VLCFQQAFWN SKGNYVVSNI NGGEYRSGKD ANSILASIHN FDPEAGCDNL
301 TFQPCSERAL ANHKAYVDSF RNLYAINKGI AQGKAVAVGR YSEDVYYNGN PWYLANFAAA
361 EQLYDAIYVW NKQGSITVTS VSLPFFRDLV SSVSTGTYSK SSSTFTNIVN AVKAYADGFI
421 EVAAKYTPSN GALAEQYDRN TGKPDSAADL TWSYSAFLSA IDRRAGLVPP SWRASVAKSQ
481 LPSTCSRIEV AGTYVAATST SFPSKQTPNP SAAPSPSPYP TACADASEVY VTFNERVSTA
541 WGETIKVVGN VPALGNWDTS KAVTLSASGY KSNDPLWSIT VPIKATGSAV QYKYIKVGTN
601 GKITWESDPN RSITLQTASS AGKCAAQTVN DSWR
SEQ ID NO: 3: amino acid sequence of mature Humicola grisea glucoamylase;
the native signal sequence is cleaved; corresponds to SEQ ID NO. 3 of
U.S. Pat. No. 7,262,041
    1 AAVDTFINTE KPIAWNKLLA NIGPNGKAAP GAAAGVVIAS PSRTDPPYFF TWTRDAALVL
  61 TGIIESLGHN YNTTLQTVIQ NYVASQAKLQ QVSNPSGTFA DGSGLGEAKF NVDLTAFTGE
121 WGRPQRDGPP LRAIALIQYA KWLIANGYKS TAKSVVWPVV KNDLAYTAQY WNETGFDLWE
181 EVPGSSFFTI ASSHRALTEG AYLAAQLDTE CRACTTVAPQ VLCFQQAFWN SKGNYVVSNI
241 NGGEYRSGKD ANSILASIHN FDPEAGCDNL TFQPCSERAL ANHKAYVDSF RNLYAINKGI
301 AQGKAVAVGR YSEDVYYNGN PWYLANFAAA EQLYDAIYVW NKQGSITVTS VSLPFFRDLV
361 SSVSTGTYSK SSSTFTNIVN AVKAYADGFI EVAAKYTPSN GALAEQYDRN TGKPDSAADL
421 TWSYSAFLSA IDRRAGLVPP SWRASVAKSQ LPSTCSRIEV AGTYVAATST SFPSKQTPNP
481 SAAPSPSPYP TACADASEVY VTFNERVSTA WGETIKVVGN VPALGNWDTS KAVTLSASGY
541 KSNDPLWSIT VPIKATGSAV QYKYIKVGTN GKITWESDPN RSITLQTASS AGKCAAQTVN
601 DSWR
SEQ ID NO: 4: Trichoderma reesei glucoamylase cDNA
<210> 4
<211> 1899
<212> DNA
<213> Trichoderma reesei
<400> 4
```

| | |
|---|---|
| atgcacgtcc tgtcgactgc ggtgctgctc ggctccgttg ccgttcaaaa ggtcctggga | 60 |
| agaccaggat caagcggtct gtccgacgtc accaagaggt ctgttgacga cttcatcagc | 120 |
| accgagacgc ctattgcact gaacaatctt ctttgcaatg ttggtcctga tggatgccgt | 180 |
| gcattcggca catcagctgg tgcggtgatt gcatctccca gcacaattga cccggactac | 240 |
| tattacatgt ggacgcgaga tagcgctctt gtcttcaaga acctcatcga ccgcttcacc | 300 |
| gaaacgtacg atgcgggcct gcagcgccgc atcgagcagt acattactgc ccaggtcact | 360 |
| ctccagggcc tctctaaccc ctcgggctcc ctcgcggacg gctctggtct cggcgagccc | 420 |
| aagtttgagt tgaccctgaa gcctttcacc ggcaactggg gtcgaccgca gcgggatggc | 480 |
| ccagctctgc gagccattgc cttgattgga tactcaaagt ggctcatcaa caacaactat | 540 |
| cagtcgactg tgtccaacgt catctggcct attgtgcgca acgacctcaa ctatgttgcc | 600 |
| cagtactgga accaaaccgg ctttgacctc tgggaagaag tcaatgggag ctcattcttt | 660 |
| actgttgcca accagcaccg agcacttgtc gagggcgcca ctcttgctgc cactcttggc | 720 |
| cagtcgggaa gcgcttattc atctgttgct ccccaggttt tgtgctttct ccaacgattc | 780 |
| tgggtgtcgt ctggtggata cgtcgactcc aacatcaaca ccaacgaggg caggactggc | 840 |
| aaggatgtca actccgtcct gacttccatc cacaccttcg atcccaacct tggctgtgac | 900 |
| gcaggcacct tccagccatg cagtgacaaa gcgctctcca acctcaaggt tgttgtcgac | 960 |
| tccttccgct ccatctacgg cgtgaacaag ggcattcctg ccggtgctgc cgtcgccatt | 1020 |
| ggccggtatg cagaggatgt gtactacaac ggcaaccctt ggtatcttgc tacatttgct | 1080 |
| gctgccgagc agctgtacga tgccatctac gtctggaaga agacgggctc catcacggtg | 1140 |
| accgccacct ccctggcctt cttccaggag cttgttcctg gcgtgacggc cgggacctac | 1200 |
| tccagcagct cttcgacctt taccaacatc atcaacgccg tctcgacata cgccgatggc | 1260 |
| ttcctcagcg aggctgccaa gtacgtcccc gccgacggtt cgctggccga gcagtttgac | 1320 |
| cgcaacagcg gcactccgct gtctgcgctt cacctgacgt ggtcgtacgc ctcgttcttg | 1380 |
| acagccacgg cccgtcgggc tggcatcgtg cccccctcgt gggccaacag cagcgctagc | 1440 |
| acgatcccct cgacgtgctc cggcgcgtcc gtggtcggat cctactcgcg tcccaccgcc | 1500 |
| acgtcattcc ctccgtcgca gacgcccaag cctggcgtgc cttccggtac tccctacacg | 1560 |
| cccctgccct gcgcgacccc aacctccgtg gccgtcacct tccacgagct cgtgtcgaca | 1620 |
| cagtttggcc agacggtcaa ggtggcgggc aacgccgcgg ccctgggcaa ctggagcacg | 1680 |
| agcgccgccg tggctctgga cgccgtcaac tatgccgata accaccccct gtggattggg | 1740 |
| acggtcaacc tcgaggctgg agacgtcgtg gagtacaagt acatcaatgt gggccaagat | 1800 |
| ggctccgtga cctgggagag tgatcccaac cacacttaca cggttcctgc ggtggcttgt | 1860 |
| gtgacgcagg ttgtcaagga ggacacctgg cagtcgtaa | 1899 |

```
SEQ ID NO: 5: Trichoderma reesei glucoamylase, full length; with signal
peptide
<210> 1
<211> 632
<212> PRT
<213> Trichoderma reesei
<400> 1
Met His Val Leu Ser Thr Ala Val Leu Leu Gly Ser Val Ala Val Gln
1 5 10 15
Lys Val Leu Gly Arg Pro Gly Ser Ser Gly Leu Ser Asp Val Thr Lys
                        20 25 30
Arg Ser Val Asp Asp Phe Ile Ser Thr Glu Thr Pro Ile Ala Leu Asn
                  35 40 45
Asn Leu Leu Cys Asn Val Gly Pro Asp Gly Cys Arg Ala Phe Gly Thr
        50 55 60
Ser Ala Gly Ala Val Ile Ala Ser Pro Ser Thr Ile Asp Pro Asp Tyr
65 70 75 80
Tyr Tyr Met Trp Thr Arg Asp Ser Ala Leu Val Phe Lys Asn Leu Ile
                                85 90 95
Asp Arg Phe Thr Glu Thr Tyr Asp Ala Gly Leu Gln Arg Arg Ile Glu
                        100 105 110
Gln Tyr Ile Thr Ala Gln Val Thr Leu Gln Gly Leu Ser Asn Pro Ser
                115 120 125
Gly Ser Leu Ala Asp Gly Ser Gly Leu Gly Glu Pro Lys Phe Glu Leu
        130 135 140
Thr Leu Lys Pro Phe Thr Gly Asn Trp Gly Arg Pro Gln Arg Asp Gly
145 150 155 160
Pro Ala Leu Arg Ala Ile Ala Leu Ile Gly Tyr Ser Lys Trp Leu Ile
                                165 170 175
Asn Asn Asn Tyr Gln Ser Thr Val Ser Asn Val Ile Trp Pro Ile Val
                        180 185 190
Arg Asn Asp Leu Asn Tyr Val Ala Gln Tyr Trp Asn Gln Thr Gly Phe
                195 200 205
Asp Leu Trp Glu Glu Val Asn Gly Ser Ser Phe Phe Thr Val Ala Asn
        210 215 220
Gln His Arg Ala Leu Val Glu Gly Ala Thr Leu Ala Ala Thr Leu Gly
225 230 235 240
Gln Ser Gly Ser Ala Tyr Ser Ser Val Ala Pro Gln Val Leu Cys Phe
                                245 250 255
Leu Gln Arg Phe Trp Val Ser Ser Gly Gly Tyr Val Asp Ser Asn Ile
                        260 265 270
Asn Thr Asn Glu Gly Arg Thr Gly Lys Asp Val Asn Ser Val Leu Thr
                275 280 285
Ser Ile His Thr Phe Asp Pro Asn Leu Gly Cys Asp Ala Gly Thr Phe
        290 295 300
Gln Pro Cys Ser Asp Lys Ala Leu Ser Asn Leu Lys Val Val Val Asp
305 310 315 320
Ser Phe Arg Ser Ile Tyr Gly Val Asn Lys Gly Ile Pro Ala Gly Ala
                                325 330 335
Ala Val Ala Ile Gly Arg Tyr Ala Glu Asp Val Tyr Tyr Asn Gly Asn
                        340 345 350
Pro Trp Tyr Leu Ala Thr Phe Ala Ala Ala Glu Gln Leu Tyr Asp Ala
                355 360 365
Ile Tyr Val Trp Lys Lys Thr Gly Ser Ile Thr Val Thr Ala Thr Ser
        370 375 380
Leu Ala Phe Phe Gln Glu Leu Val Pro Gly Val Thr Ala Gly Thr Tyr
385 390 395 400
Ser Ser Ser Ser Ser Thr Phe Thr Asn Ile Ile Asn Ala Val Ser Thr
                                405 410 415
Tyr Ala Asp Gly Phe Leu Ser Glu Ala Ala Lys Tyr Val Pro Ala Asp
                        420 425 430
Gly Ser Leu Ala Glu Gln Phe Asp Arg Asn Ser Gly Thr Pro Leu Ser
                435 440 445
Ala Leu His Leu Thr Trp Ser Tyr Ala Ser Phe Leu Thr Ala Thr Ala
        450 455 460
Arg Arg Ala Gly Ile Val Pro Pro Ser Trp Ala Asn Ser Ser Ala Ser
465 470 475 480
Thr Ile Pro Ser Thr Cys Ser Gly Ala Ser Val Val Gly Ser Tyr Ser
                                485 490 495
Arg Pro Thr Ala Thr Ser Phe Pro Pro Ser Gln Thr Pro Lys Pro Gly
                        500 505 510
Val Pro Ser Gly Thr Pro Tyr Thr Pro Leu Pro Cys Ala Thr Pro Thr
                515 520 525
Ser Val Ala Val Thr Phe His Glu Leu Val Ser Thr Gln Phe Gly Gln
        530 535 540
Thr Val Lys Val Ala Gly Asn Ala Ala Ala Leu Gly Asn Trp Ser Thr
545 550 555 560
Ser Ala Ala Val Ala Leu Asp Ala Val Asn Tyr Ala Asp Asn His Pro
                                565 570 575
Leu Trp Ile Gly Thr Val Asn Leu Glu Ala Gly Asp Val Val Glu Tyr
                        580 585 590
Lys Tyr Ile Asn Val Gly Gln Asp Gly Ser Val Thr Trp Glu Ser Asp
                595 600 605
Pro Asn His Thr Tyr Thr Val Pro Ala Val Ala Cys Val Thr Gln Val
        610 615 620
Val Lys Glu Asp Thr Trp Gln Ser
625 630
SEQ ID NO: 6: Trichoderma reesei glucoamylase, mature protein; without
signal peptide
<210> 2
<211> 599
<212> PRT
<213> Trichoderma reesei
<400> 2
Ser Val Asp Asp Phe Ile Ser Thr Glu Thr Pro Ile Ala Leu Asn Asn
1 5 10 15
Leu Leu Cys Asn Val Gly Pro Asp Gly Cys Arg Ala Phe Gly Thr Ser
                        20 25 30
Ala Gly Ala Val Ile Ala Ser Pro Ser Thr Ile Asp Pro Asp Tyr Tyr
                35 40 45
Tyr Met Trp Thr Arg Asp Ser Ala Leu Val Phe Lys Asn Leu Ile Asp
        50 55 60
Arg Phe Thr Glu Thr Tyr Asp Ala Gly Leu Gln Arg Arg Ile Glu Gln
65 70 75 80
Tyr Ile Thr Ala Gln Val Thr Leu Gln Gly Leu Ser Asn Pro Ser Gly
                                85 90 95
Ser Leu Ala Asp Gly Ser Gly Leu Gly Glu Pro Lys Phe Glu Leu Thr
                        100 105 110
Leu Lys Pro Phe Thr Gly Asn Trp Gly Arg Pro Gln Arg Asp Gly Pro
                115 120 125
Ala Leu Arg Ala Ile Ala Leu Ile Gly Tyr Ser Lys Trp Leu Ile Asn
        130 135 140
Asn Asn Tyr Gln Ser Thr Val Ser Asn Val Ile Trp Pro Ile Val Arg
145 150 155 160
Asn Asp Leu Asn Tyr Val Ala Gln Tyr Trp Asn Gln Thr Gly Phe Asp
                                165 170 175
Leu Trp Glu Glu Val Asn Gly Ser Ser Phe Phe Thr Val Ala Asn Gln
                        180 185 190
His Arg Ala Leu Val Glu Gly Ala Thr Leu Ala Ala Thr Leu Gly Gln
                195 200 205
Ser Gly Ser Ala Tyr Ser Ser Val Ala Pro Gln Val Leu Cys Phe Leu
        210 215 220
Gln Arg Phe Trp Val Ser Ser Gly Gly Tyr Val Asp Ser Asn Ile Asn
225 230 235 240
Thr Asn Glu Gly Arg Thr Gly Lys Asp Val Asn Ser Val Leu Thr Ser
                                245 250 255
Ile His Thr Phe Asp Pro Asn Leu Gly Cys Asp Ala Gly Thr Phe Gln
                        260 265 270
Pro Cys Ser Asp Lys Ala Leu Ser Asn Leu Lys Val Val Val Asp Ser
                275 280 285
Phe Arg Ser Ile Tyr Gly Val Asn Lys Gly Ile Pro Ala Gly Ala Ala
        290 295 300
Val Ala Ile Gly Arg Tyr Ala Glu Asp Val Tyr Tyr Asn Gly Asn Pro
305 310 315 320
Trp Tyr Leu Ala Thr Phe Ala Ala Ala Glu Gln Leu Tyr Asp Ala Ile
                                325 330 335
Tyr Val Trp Lys Lys Thr Gly Ser Ile Thr Val Thr Ala Thr Ser Leu
                        340 345 350
Ala Phe Phe Gln Glu Leu Val Pro Gly Val Thr Ala Gly Thr Tyr Ser
                355 360 365
Ser Ser Ser Ser Thr Phe Thr Asn Ile Ile Asn Ala Val Ser Thr Tyr
        370 375 380
Ala Asp Gly Phe Leu Ser Glu Ala Ala Lys Tyr Val Pro Ala Asp Gly
385 390 395 400
Ser Leu Ala Glu Gln Phe Asp Arg Asn Ser Gly Thr Pro Leu Ser Ala
                                405 410 415
Leu His Leu Thr Trp Ser Tyr Ala Ser Phe Leu Thr Ala Thr Ala Arg
                        420 425 430
Arg Ala Gly Ile Val Pro Pro Ser Trp Ala Asn Ser Ser Ala Ser Thr
                435 440 445
Ile Pro Ser Thr Cys Ser Gly Ala Ser Val Val Gly Ser Tyr Ser Arg
        450 455 460
Pro Thr Ala Thr Ser Phe Pro Pro Ser Gln Thr Pro Lys Pro Gly Val
465 470 475 480
Pro Ser Gly Thr Pro Tyr Thr Pro Leu Pro Cys Ala Thr Pro Thr Ser
                                485 490 495
Val Ala Val Thr Phe His Glu Leu Val Ser Thr Gln Phe Gly Gln Thr
                        500 505 510
Val Lys Val Ala Gly Asn Ala Ala Ala Leu Gly Asn Trp Ser Thr Ser
                515 520 525
Ala Ala Val Ala Leu Asp Ala Val Asn Tyr Ala Asp Asn His Pro Leu
        530 535 540
Trp Ile Gly Thr Val Asn Leu Glu Ala Gly Asp Val Val Glu Tyr Lys
545 550 555 560
Tyr Ile Asn Val Gly Gln Asp Gly Ser Val Thr Trp Glu Ser Asp Pro
                                565 570 575
Asn His Thr Tyr Thr Val Pro Ala Val Ala Cys Val Thr Gln Val Val
                        580 585 590
Lys Glu Asp Thr Trp Gln Ser
                595
SEQ ID NO: 7: Trichoderma reesei glucoamylase catalytic domain, 1-453 of
mature TrGA, catalytic domain
<210> 3
<211> 453
<212> PRT
<213> Trichoderma reesei
<400> 3
Ser Val Asp Asp Phe Ile Ser Thr Glu Thr Pro Ile Ala Leu Asn Asn
1 5 10 15
Leu Leu Cys Asn Val Gly Pro Asp Gly Cys Arg Ala Phe Gly Thr Ser
                        20 25 30
Ala Gly Ala Val Ile Ala Ser Pro Ser Thr Ile Asp Pro Asp Tyr Tyr
                35 40 45
Tyr Met Trp Thr Arg Asp Ser Ala Leu Val Phe Lys Asn Leu Ile Asp
        50 55 60
Arg Phe Thr Glu Thr Tyr Asp Ala Gly Leu Gln Arg Arg Ile Glu Gln
65 70 75 80
Tyr Ile Thr Ala Gln Val Thr Leu Gln Gly Leu Ser Asn Pro Ser Gly
                                  85 90 95
Ser Leu Ala Asp Gly Ser Gly Leu Gly Glu Pro Lys Phe Glu Leu Thr
                        100 105 110
Leu Lys Pro Phe Thr Gly Asn Trp Gly Arg Pro Gln Arg Asp Gly Pro
                115 120 125
Ala Leu Arg Ala Ile Ala Leu Ile Gly Tyr Ser Lys Trp Leu Ile Asn
        130 135 140
Asn Asn Tyr Gln Ser Thr Val Ser Asn Val Ile Trp Pro Ile Val Arg
145 150 155 160
Asn Asp Leu Asn Tyr Val Ala Gln Tyr Trp Asn Gln Thr Gly Phe Asp
                                165 170 175
Leu Trp Glu Glu Val Asn Gly Ser Ser Phe Phe Thr Val Ala Asn Gln
                        180 185 190
His Arg Ala Leu Val Glu Gly Ala Thr Leu Ala Ala Thr Leu Gly Gln
                195 200 205
Ser Gly Ser Ala Tyr Ser Ser Val Ala Pro Gln Val Leu Cys Phe Leu
        210 215 220
Gln Arg Phe Trp Val Ser Ser Gly Gly Tyr Val Asp Ser Asn Ile Asn
225 230 235 240
Thr Asn Glu Gly Arg Thr Gly Lys Asp Val Asn Ser Val Leu Thr Ser
                                245 250 255
Ile His Thr Phe Asp Pro Asn Leu Gly Cys Asp Ala Gly Thr Phe Gln
                        260 265 270
Pro Cys Ser Asp Lys Ala Leu Ser Asn Leu Lys Val Val Val Asp Ser
                275 280 285
Phe Arg Ser Ile Tyr Gly Val Asn Lys Gly Ile Pro Ala Gly Ala Ala
        290 295 300
Val Ala Ile Gly Arg Tyr Ala Glu Asp Val Tyr Tyr Asn Gly Asn Pro
305 310 315 320
Trp Tyr Leu Ala Thr Phe Ala Ala Ala Glu Gln Leu Tyr Asp Ala Ile
                                325 330 335
Tyr Val Trp Lys Lys Thr Gly Ser Ile Thr Val Thr Ala Thr Ser Leu
                        340 345 350
Ala Phe Phe Gln Glu Leu Val Pro Gly Val Thr Ala Gly Thr Tyr Ser
                355 360 365
Ser Ser Ser Ser Thr Phe Thr Asn Ile Ile Asn Ala Val Ser Thr Tyr
        370 375 380
Ala Asp Gly Phe Leu Ser Glu Ala Ala Lys Tyr Val Pro Ala Asp Gly
385 390 395 400
Ser Leu Ala Glu Gln Phe Asp Arg Asn Ser Gly Thr Pro Leu Ser Ala
                                405 410 415
Leu His Leu Thr Trp Ser Tyr Ala Ser Phe Leu Thr Ala Thr Ala Arg
                        420 425 430
Arg Ala Gly Ile Val Pro Pro Ser Trp Ala Asn Ser Ser Ala Ser Thr
                435 440 445
Ile Pro Ser Thr Cys
        450
SEQ ID NO: 8: native RhGA (P07683.2 GI: 1168453)
    1 mqlfnlplkv sfflvlsyfs llvsaasips sasvqldsyn ydgstfsgki yvkniayskk
  61 vtviyadgsd nwnnngntia asysapisgs nyeywtfsas ingikefyik yevsgktyyd
121 nnnsanyqvs tskpttttat attttapsts tttppsrsep atfptgnsti sswikkqegi
181 srfamlrnin ppgsatgfia aslstagpdy yyawtrdaal tsnvivyeyn ttlsgnktil
241 nvlkdyvtfs vktqststvc nclgepkfnp dasgytgawg rpqndgpaer attfilfads
301 yltqtkdasy vtgtlkpaif kdldyvvnvw sngcfdlwee vngvhfytlm vmrkglllga
361 dfakrngdst rastysstas tiankissfw vssnnwiqvs qsvtggvskk gldvstllaa
421 nlgsvddgff tpgsekilat avavedsfas lypinknlps ylgnsigryp edtyngngns
481 qgnswflavt gyaelyyrai kewignggvt vssislpffk kfdssatsgk kytvgtsdfn
541 nlaqnialaa drflstvqlh ahnngslaee fdrttglstg ardltwshas litasyakag
601 apaa
SEQ ID NO: 9: mature RhGA (P07683.2 GI: 1168453)
    1 asipssasvq ldsynydgst fsgkiyvkni ayskkvtviy adgsdnwnnn gntiaasysa
  61 pisgsnyeyw tfsasingik efyikyevsg ktyydnnnsa nyqvstskpt tttatatttt
121 apststttpp srsepatfpt gnstisswik kqegisrfam lrninppgsa tgfiaaslst
181 agpdyyyawt rdaaltsnvi vyeynttlsg nktilnvlkd yvtfsvktqs tstvcnclge
241 pkfnpdasgy tgawgrpqnd gpaerattfi lfadsyltqt kdasyvtgtl kpaifkdldy
301 vvnvwsngcf dlweevngvh fytlmvmrkg lllgadfakr ngdstrasty sstastiank
361 issfwvssnn wiqvsqsvtg gvskkgldvs tllaanlgsv ddgfftpgse kilatavave
421 dsfaslypin knlpsylgns igrypedtyn gngnsqgnsw flavtgyael yyraikewig
481 nggvtvssis lpffkkfdss atsgkkytvg tsdfnnlaqn ialaadrfls tvqlhahnng
541 slaeefdrtt glstgardlt wshaslitas yakagapaa
```

1. A method for producing isoprene comprising culturing a host cell, which comprises a heterologous nucleic acid encoding an isoprene synthase polypeptide, and saccharifying and fermenting a starch substrate under simultaneous saccharification and fermentation (SSF) conditions in the presence of a glucoamylase, wherein the saccharification and fermentation are performed at pH 6.5 to 8.0, wherein the glucoamylase possesses at least 50% activity at pH 6.0 or above relative to its maximum activity, wherein the glucoamylase is Trichoderma reesei glucoamylase (TrGA) comprising SEQ ID NO: 6, or a variant thereof, and wherein the variant has at least 99% sequence identity to the parent glucoamylase, wherein the host cell is selected from the group consisting of bacterial cells and fungal cells.

2. The method of claim 1, wherein the variant has one amino acid modification compared to the parent glucoamylase.

3-4. (canceled)

5. The method of claim 1, wherein the TrGA is SEQ ID No: 6.

6. (canceled)

7. The method of claim 1, wherein SSF are carried out at pH 6.5 to 7.5.

8. The method of claim 1, wherein SSF are carried out at pH 7.0 to 7.5.

9. The method of claim 1, wherein SSF is performed at a temperature in a range of about 30° C. to about 60° C.

10. The method of claim 9, wherein SSF is performed at a temperature in a range of about 40° C. to about 60° C.

11. The method of claim 1, wherein the starch substrate is about 15% to 50% dry solid (DS).

12. The method of claim 1, wherein the starch substrate is about 15% to 30% dry solid (DS).

13. The method of claim 1, wherein the starch substrate is about 15% to 25% dry solid (DS).

14. The method of claim 1, wherein the starch substrate is granular starch or liquefied starch.

15. The method of claim 1, wherein the glucoamylase is dosed at a range of about 0.1 to about 2.0 GAU per gram of dry substance starch.

16. The method of claim 15, wherein the glucoamylase is dosed at a range of about 0.2 to about 1.0 GAU per gram of dry substance starch.

17. The method of claim 15, wherein the glucoamylase is dosed at a range of about 0.5 to 1.0 GAU per gram of dry substance starch.

18. The method of claim 1 further comprising adding an alpha-amylase.

19. The method of claim 18, wherein the alpha-amylase is from a Bacillus species, or a variant thereof.

20. The method of claim 19, wherein the alpha-amylase is a Bacillus subtilis alpha-amylase (AmyE), a Bacillus amyloliquefaciens alpha-amylase, a Bacillus licheniformis alpha-amylase, a Bacillus stearothermophilus alpha-amylase, or a variant thereof.

21. The method of claim 1, wherein the starch substrate is from corn, wheat, rye, barley, sorghum, cassava, tapioca, and any combination thereof.

22. The method of claim 1 wherein the heterologous nucleic acid is operably linked to a promoter and wherein the production of isoprene by the cells is greater than about 5 g/L.

23. The method of claim 1 wherein the isoprene synthase polypeptide is a plant isoprene synthase polypeptide.

24. The method of claim 23 wherein the plant isoprene synthase is selected from the group consisting of Pueraria montana, Pueraria lobata, Populus alba, Populus nigra, Populus trichocarpa, Populus alba x tremula, Populus tremuloides and Quercus robur.

25. The method of claim 1 wherein the host cells further comprise one or more heterologous nucleic acid encoding a mevalonate (MVA) pathway polypeptide or a DXP pathway polypeptide.

26. The method of claim 1 wherein the host cells further comprise (i) one or more non-modified nucleic acids encoding feedback-resistant mevalonate kinase polypeptides or (ii) one or more additional copies of an endogenous nucleic acid encoding a feedback-resistant mevalonate kinase polypeptide.

27. The method of claim 26 wherein the feedback-resistant mevalonate kinase is archaeal mevalonate kinase.

28. The method of claim 26 wherein the mevalonate kinase polypeptide is selected from the group consisting of M. mazei, Lactobacillus mevalonate kinase polypeptide, Lactobacillus sakei mevalonate kinase polypeptide, yeast mevalonate kinase polypeptide, Streptococcus mevalonate kinase polypeptide, Streptococcus pneumoniae mevalonate kinase polypeptide, Streptomyces mevalonate kinase polypeptide, and Streptomyces CL190 mevalonate kinase polypeptide.

29. (canceled)

30. The method of claim 1 wherein the bacterial cells are selected from the group consisting of gram-positive bacterial cells, gram-negative bacterial cells, E. coli, P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., and P. alcaligenes cells.

31. The method of claim 1 wherein the fungal cells are selected from the group consisting of Aspergillus, yeast, Trichoderma, or Yarrowia cells.

32. The method of claim 31 wherein the yeast is Saccharomyces sp., Schizosaccharomyces sp., Pichia sp., Candida sp. or Y. lipolytica cells.

33. The method of claim 31 wherein the fungal cells are selected from the group consisting of A. oryzae, A. niger, S. cerevisiae, S. pombe, T. reesei, H. insolens, H. lanuginose, H. grisea, C. lucknowense, A. oryzae, A. niger, A sojae, A. japonicus, A. nidulans, A. aculeatus, A. awamori, F. roseum, F. graminum F. cerealis, F. oxysporuim, F. venenatum, N. crassa, M. miehei, T. viride, F. oxysporum, and F. solan cells.

34-35. (canceled)

36. The method of claim 1 wherein the host cells are grown under conditions that decouple isoprene production from cell growth.

37. The method of claim 1 wherein the host cells are grown under limited glucose conditions.

38. A system for producing isoprene comprising (i) a bioreactor within which saccharification and fermentation are performed at pH 6.5 to 8.0; (ii) a host cell comprising a heterologous nucleic acid encoding an isoprene synthase polypeptide; (iii) a glucoamylase that possesses at least 50% activity at pH 6.0 or above relative to its maximum activity, wherein the glucoamylase is Trichoderma reesei glucoamylase (TrGA) comprising SEQ ID NO: 6, or a variant thereof, and wherein the variant has at least 99% sequence identity to the parent glucoamylase, wherein the host cell is selected from the group consisting of bacterial cells and fungal cells.

39. A method for producing isoprene comprising culturing a host cell, which comprises a heterologous nucleic acid encoding an isoprene synthase polypeptide, and saccharifying and fermenting a starch substrate under simultaneous saccharification and fermentation (SSF) conditions in the presence of a glucoamylase and at least one other enzyme, wherein the saccharification and fermentation are performed at pH 6.5 to 8.0, wherein the glucoamylase possesses at least 50% activity at pH 6.0 or above relative to its maximum activity, wherein the glucoamylase is Trichoderma reesei glucoamylase (TrGA) comprising SEQ ID NO: 6, or a variant thereof, and wherein the variant has at least 99% sequence identity to a parent glucoamylase, and wherein the other enzyme is selected from the group consisting of proteases, pullulanases, isoamylases, cellulases, hemicellulases, xylanases, cyclodextrin glycotransferases, lipases, phytases, laccases, oxidases, esterases, cutinases, xylanases, and alpha-glucosidases, wherein the host cell is selected from the group consisting of bacterial cells and fungal cells.

40. A method for producing isoprene comprising culturing a host cell, which comprises a heterologous nucleic acid encoding an isoprene synthase polypeptide, and saccharifying and fermenting a starch substrate under simultaneous saccharification and fermentation (SSF) conditions in the presence of a glucoamylase and at least one other non-starch polysaccharide hydrolyzing enzyme, wherein the saccharification and fermentation are performed at pH 6.5 to 8.0, wherein the glucoamylase possesses at least 50% activity at pH 6.0 or above relative to its maximum activity, wherein the glucoamylase is Trichoderma reesei glucoamylase (TrGA) comprising SEQ ID NO: 6, or a variant thereof, and wherein the variant has at least 99% sequence identity to a parent glucoamylase, and wherein the non-starch polysaccharide hydrolyzing enzyme is selected from the group consisting of cellulases, hemicellulases and pectinases, wherein the host cell is selected from the group consisting of bacterial cells and fungal cells.
